(19) United States
(12) Patent Application Publication
(10) Pub. No.: US 2015/0079590 A1
(43) Pub. Date: Mar. 19, 2015

(54) CHARACTERIZATION AND ANALYSIS OF THE COMPOSITION AND DYNAMICS OF THE MAMMALIAN RIBOPROTEOME

(71) Applicant: Beth Israel Deaconess Medical Center, Inc., Boston, MA (US)

(72) Inventors: Pier Paolo PANDOLFI, Boston, MA (US); Markus RESCHKE, Boston, MA (US); John CLOHESSY, Boston, MA (US)

(21) Appl. No.: 14/489,018

(22) Filed: Sep. 17, 2014

Related U.S. Application Data
(60) Provisional application No. 61/879,565, filed on Sep. 18, 2013.

(51) Int. Cl. G01N 33/574

(52) U.S. Cl. CPC G01N 33/57407; G01N 2500/04; G01N 2800/50
USPC 435/6.11; 436/501; 435/7.1

(57) ABSTRACT

The present invention relates to methods for identifying compounds that modulate the translation machinery and methods of diagnosing cancer related to deregulation of the translation machinery. In particular, the methods and diagnostic tests include determining an association between one or more targets and one or more components of the riboproteome and treating a subject with cancer by administering a compound that modulates the association between one or more targets and one or more components of the riboproteome.

### CROSS-REFERENCE TO RELATED APPLICATIONS

This application claims the benefit of U.S. Provisional Application No. 61/879,565, filed Sep. 18, 2013, hereby incorporated by reference in its entirety.

### STATEMENT AS TO FEDERALLY SPONSORED RESEARCH

This invention was made with government support under grant number RC1 DK087679 awarded by the National Cancer Institute. The government has certain rights in this invention.

### BACKGROUND OF THE INVENTION

The present invention relates to methods for identifying compounds that modulate the translation machinery and methods of diagnosing cancer related to deregulation of the translation machinery.

Increasing evidence points to an important role for the ribosome in the regulation of biological processes, and as a target for deregulation in disease. Key regulators of translation are specifically targeted in human diseases, including cancer. Indeed, recent data suggest that RNA binding proteins (RBPs) are frequently associated with disease. Additionally, deficiency and mutation of ribosome and ribosome biogenesis proteins themselves are associated with disease and developmental abnormalities, including Diamond-Blackfan anemia (DBA), Shwachman-Diamond syndrome (SDS) and X-linked dyskeratosis congenita (X-DC). However, a more global approach to systematically determine in greater detail the players that coordinate translation is currently lacking. Such an approach will in turn enable the identification of key regulators of translation in specific conditions, and help better elucidate the role that these proteins play in disease pathology and to identify novel markers and targets for disease diagnosis and therapy.

### SUMMARY OF THE INVENTION

The invention features a method of identifying a compound for the treatment of cancer, the method including: a) providing one or more targets and one or more components of the riboproteome known to associate with the target, b) contacting the combined materials of a) with a compound, and c) monitoring an alteration in the target or the component of the riboproteome, wherein a modulation in the association between the target and the component of the riboproteome identifies the compound as a potential therapeutic for the treatment of cancer. In certain embodiments, the alteration is seen in both the target and the component of the riboproteome. In other embodiments, the alteration is in the association between the target and the component of the riboproteome.

The invention also features a method of diagnosing a subject as having, or having a predisposition to a particular type of cancer, the method including a) determining an association between one or more targets and one or more components of the riboproteome in the subject, and b) comparing the level of association to a normal reference, wherein a change in the level of association diagnoses the subject as having a particular type of cancer, and c) treating the subject for the particular type of cancer by administering a compound that modulates the association between the target and the component of the riboproteome.

In all aspects of the invention, the cancer is a cancer resulting from a change in the level of association between the target and the component of the riboproteome. In particular embodiments, the cancer resulting from the change in the level of the association between the target and the component of the riboproteome is selected from the group consisting of: acute myeloid leukemia, adenoid cystic carcinoma, bladder cancer, bladder urothelial carcinoma, brain lower grade glioma, breast invasive carcinoma, cervical squamous cell carcinoma and endocervical adenocarcinoma, colon and rectum adenocarcinoma, glioblastoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large b-cell lymphoma, ovarian serous cystadenocarcinoma, pancreatic adenocarcinoma, prostate adenocarcinoma, sarcoma, skin cutaneous melanoma, stomach adenocarcinoma, thyroid carcinoma, and uterine corpus endometrial carcinoma.

In one aspect, the one or more targets is selected from the group consisting of: a RNA binding protein, a metabolic enzyme, a cell adhesion molecule, a component of the proteasome, a component of the ubiquitination machinery, a component of the neddylation machinery, and a signaling protein. In certain embodiments, the one or more targets is a target listed in Table 1 or Table 2.

In a second aspect, the one or more components of the riboproteome is a component listed in Table 3 or Table 4.

In a third aspect, the compound is a small molecule, an RNAi agent, a soluble polypeptide, an antibody, or an antigen-binding fragment. In one embodiment, the compound specifically interacts with the one or more targets. In another embodiment, the compound inhibits or promotes the activity of the one or more targets. Ina third embodiment, the compound specifically interacts with the one or more components of the riboproteome. In a fourth embodiment, the compound interacts with a secondary target to modulate the association between the target and the component of the riboproteome.

In certain aspects of the invention, the one or more targets is myristoylated alanine-rich C kinase substrate (MARCKS) or La-related protein 1 (LARP1). In other embodiments, the particular type of cancer is prostate adenocarcinoma.

Finally, the invention also features a method of diagnosing a subject as having, or having a predisposition to a particular type of cancer or predicting a response to treatment of a particular type of cancer, the method including: a) obtaining a riboproteome profile from the subject, b) identifying changes in the riboproteome profile compared to a normal reference, and c) treating the subject or providing an improved treatment regime based on the changes in the riboproteome profile.

### DEFINITIONS

By “riboproteome” is meant the proteins that constitute the actively translating ribosome. The proteins of the riboproteome can associate with (i) the ribosome itself, and which may be required for either directing translation or quality control of nascent proteins, or (ii) mRNAs undergoing active translation.

By “riboproteome profile” is meant the genes, proteins, combination of genes, combination of proteins present in the riboproteome that make up the entire landscape of the riboproteome in a subject.

By “changes in the riboproteome profile” is meant a reduction, amplification, appearance, or disappearance in a gene, protein, combinations of genes, or combinations of proteins found in the riboproteome profile when compared to a normal reference. Changes in the riboproteome profile may represent indicators that can help to classify and/or stratify specific patient subgroups that in turn can provide a diagnosis and/or prognosis for treatment of a cancer resulting from a change in the level of association between the target and the component of the riboproteome.

By “an alteration in the target or the component of the riboproteome” is meant a reduction, amplification, appearance, or disappearance of the target or the component of the riboproteome.

By “association between the target and the component of the riboproteome” is meant a physical interaction between a target and a component, or an interaction that is mediated by another macromolecule.

By “modulation or modulates” is meant an increase or decrease in association between the target and the component of the riboproteome as compared to a control. A compound that modulates the association between the target and the component of the riboproteome can affect upstream or downstream signaling events that lead to modulation of the association between the target and the component of the riboproteome or directly affect the association between the target and the component of the riboproteome itself.

By “a change in the level of association” is meant an increase or decrease in the gene expression, protein expression, and/or activity of the target or the component of the riboproteome which leads to an increase or decrease in association of the target to the component of the riboproteome when compared to a control (e.g., a decrease of at least 2-fold, e.g., from about 2-fold to about 150-fold, e.g., from 5-fold to 150-fold, from 5-fold to 100-fold, from 10-fold to 150-fold, from 10-fold to 100-fold, from 50-fold to 150-fold, from 50-fold to 100-fold, from 75-fold to 150-fold, or from 75-fold to 100-fold, as compared to a control, an increase of at least 2-fold, e.g., from about 2-fold to about 150-fold, e.g., from 5-fold to 150-fold, from 5-fold to 100-fold, from 10-fold to 150-fold, from 10-fold to 100-fold, from 50-fold to 150-fold, from 50-fold to 100-fold, from 75-fold to 150-fold, or from 75-fold to 100-fold, as compared to a control).

By “specifically interacts” is meant a compound that produces a desired effect in one macromolecule (e.g., the one or more targets) without influencing and/or creating adverse effects in other macromolecules and subtypes thereof. Specific interaction can be determined by binding kinetics (e.g., degree of interaction between a compound with a macromolecule, e.g., an increase in ligand-receptor affinity, e.g., a decrease in dissociation constant (K_{D}) of 2-fold, 3-fold, 4-fold, 5-fold, 10-fold, 20-fold, 30-fold, 100-fold, or 200-fold, a decrease dose (e.g., a decrease by 5%, 10%, 15%, 20%, 25%, 30%, 35%, 40%, 45%, 50%, 55%, 60%, 65%, 70%, 75%, 80%, 85%, 90%, or 95%) of the compound compared to a reference compound). Specific interaction can also be a measure of local accumulation of a compound in the macromolecule compared to accumulation in other macromolecules and subtypes thereof (e.g., an increase in accumulation by 5%, 10%, 15%, 20%, 25%, 30%, 35%, 40%, 45%, 50%, 55%, 60%, 65%, 70%, 75%, 80%, 85%, 90%, or 95% at the desired macromolecule compared to a reference agent or compound). Specific interaction can also be an increase in efficacy and/or potency of a compound (e.g., an increase by 5%, 10%, 15%, 20%, 25%, 30%, 35%, 40%, 45%, 50%, 55%, 60%, 65%, 70%, 75%, 80%, 85%, 90%, or 95% compared to a reference compound).

By “inhibits the activity” is meant a compound that decreases or reduces gene expression, protein expression, or activity (e.g., enzymatic activity) of the target, as defined herein, compared to a control (e.g., a decrease by 5%, 10%, 15%, 20%, 25%, 30%, 35%, 40%, 45%, 50%, 55%, 60%, 65%, 70%, 75%, 80%, 85%, 90%, or 95%, as compared to a control or a normal reference sample). By “promotes the activity” is meant a compound that increases or enhances gene expression, protein expression, or activity (e.g., enzymatic activity) of the target, as defined herein, compared to a control (e.g., an increase of 5%, 10%, 15%, 20%, 25%, 30%, 35%, 40%, 45%, 50%, 55%, 60%, 65%, 70%, 75%, 80%, 85%, 90%, or 95%, as compared to a control or a normal reference sample).

By “secondary target” is meant a target that does not associates with the components of the riboproteome. A secondary target may be a protein involved in a signaling pathway associated with cancer (e.g., mTOR, TORC1, TORC2, Ras, AKT, and PI3K)

By “normal reference” is meant any sample, standard, standard curve, or level that is used for comparison purposes. A “normal reference” can be, for example, a prior sample taken from the same subject; a sample from a normal healthy subject; a sample from a subject not having a cancer resulting from the change in level of association between a target and a component of the riboproteome, or a sample from a subject that has been treated for a cancer resulting from the change in level of association between a target and a component of the riboproteome.

By “RNAi agent” is meant any agent or compound that exerts a gene silencing effect by hybridizing a target nucleic acid. RNAi agents include any nucleic acid molecules that are capable of mediating sequence-specific RNAi (e.g., under stringent conditions), for example, a short interfering RNA (sRNA), double-stranded RNA (dsRNA), microRNA (miRNA), short hairpin RNA (shRNA), short interfering oligonucleotide, short interfering nucleic acid, short interfering modified oligonucleotide, short interfering nucleic acid, short interfering modified oligonucleotide, chemically-modified siRNA, post-transcriptional gene silencing RNA (ptgsRNA), and Dicer-substrate RNA (DsiRNA).

By “treating” is meant obtaining beneficial or desired results, such as clinical results. Beneficial or desired results can include, but are not limited to, alleviation or amelioration of one or more symptoms or conditions; diminishment of extent of disease, disorder, or condition; stabilization (i.e., not worsening) of a state of disease, disorder, or condition; prevention of spread of disease, disorder, or condition; delay or slowing the progress of the disease, disorder, or condition; amelioration or palliation of the disease, disorder, or condition; and remission (whether partial or total), whether detectable or undetectable.

By “amount sufficient” of an agent is meant the amount of the agent sufficient to effect beneficial or desired result (e.g., treatment of a cancer resulting from a change in the level of association of a target and component of the riboproteome), as compared to the response obtained without administration of the composition.

By “subject” is meant a mammal (e.g., a human).

### BRIEF DESCRIPTION OF THE DRAWINGS

FIGS. 1A-1N show a quantitative riboproteomics approach to study the composition of riboproteomes. (FIG. 1A) Schematic representation of the SILAC-based mass spectrometry experiments. (FIG. 1B) Scatter blot with normalized Log₂ (H/L) ratios/Log₁₀ intensities highlighting the distribution of all quantified proteins between Du145 H and L labeled cells. Note that most of the proteins have a ratio of 1:1 between the light and heavy state and therefore have a value close to 0 on a Logₑ axis (Mean 0.0229; Std. Dev. 0.1866). (FIG. 1C) Standard scatter blot with normalized Log₂ (H/L) ratios comparing the two N/C datasets (RWPE1 vs. Du145 and PWR1 E vs. Du145). All shared proteins between the datasets are plotted. Both datasets show a highly significant positive correlation (R²=0.4662; p=<0.0001). (FIGS. 1D, 1E and 1F) Standard scatter blots with normalized Log₂ (H/L) ratios/Log₁₀ Intensities (Normal vs. Cancer n=3, left panel; Cancer vs. Cancer n=3, middle panel; PPC1 DMSO vs. PP242, n=3, right panel) highlighting the distribution of quantified proteins in each screen (cut off values for enriched proteins was 2 standard deviations (2σ) from the mean—dashed red lines). Proteins of interest in either experimental setting are highlighted. (FIG. 1G). Density gradient centrifugation of polysomes. (FIG. 1H). Protease activity in polysomal fractions. (FIG. 1I). Comparison of riboproteomes in different cell lines. (FIG. 1J) differences between N/C riboproteome data sets. (FIG. 1K). Comparison of cancer cell lines treated with DMSO and rapamycin. (FIG. 1L). Comparison of riboproteome from Npm1 wild type and null immortalized MEFs. (FIG. 1M). Number of proteins identified using SILAC-based approach. (FIG. 1N). Plot of proteins of the large and small subunit of the ribosome in different cell lines.

FIGS. 2A-2M show analysis of the prostate riboproteome. (FIG. 2A) Venn diagram showing how proteins identified in each of the 5 SILAC experiments utilizing prostate cell lines are shared between each of the individual datasets. Out of total of 1,499 proteins quantified between all experiments, 363 are shared by all 5 experiments. (FIG. 2B) Pie chart illustrating the distribution of proteins identified across the various experiments. Proteins identified in a single experiment (30%) are highlighted by the detached blue pie slice, while proteins identified in all 5 experiments (24%) are indicated by a bold border. (FIG. 2C) Gene ontology (GO) analysis of the prostate riboproteome highlights multiple different pathways and functional groups that are significantly enriched in the riboproteome. A table of significantly enriched GO terms relating to translation identified by DAVID analysis is shown. (FIG. 2D) Venn diagram indicating the extent of overlap between the RNA binding protein (RBP) interactome identified by Castello et al. and the riboproteome constituents described here. (FIG. 2E) Pie charts to illustrate the extent to which components of the RBP interactome overlap with the riboproteome. The left panel shows the percentage of identified proteins that are also called within the Castello et al. dataset for the core riboproteomic dataset (i.e. identified in all 5 experiments). Right panel illustrates how the RBP-interactome components identified in the riboproteomic dataset are distributed amongst the various RBP-interactome categories defined by Castello et al. (FIG. 2F). Proteins quantified in experiments. (FIGS. 2G, 2H). Comparison of datasets between different cell lines. (FIG. 2I). Enrichment of proteins related to protein synthesis, post-translational modification and protein folding. (FIG. 2J). Pathway analysis demonstrated EIF2 signaling, regulation of eIF4 and p70S6K signaling, and mTOR signaling pathways to be significantly represented. (FIG. 2K). KEGG pathway analysis of proteins identified in all 5 experimental datasets (363/1499) compared to proteins identified in at least 1 experiment (1499). (FIG. 2L). RBP proteins identified by Castello et al. and distribution of RBP-interactome. (FIG. 2M). Riboproteome breakdown and RBP breakdown quantified in only 1 experiment.

FIGS. 3A-3M show alterations to the riboproteome in cancer. (FIGS. 3A, 3G) Forest plot highlighting the enrichment of amplifications amongst riboproteomic genes when compared to background genes in the cBio TCGA dataset. (FIGS. 3B, 3H) A similar forest plot demonstrating significantly less heterozygous deletions amongst riboproteomic genes in the cBio TCGA dataset when compared to background genes. (FIGS. 3C, 3I) Circos plot to illustrate the distribution of all riboproteomic components across the genome. Light shaded bars represent individual riboproteomic components, and their genomic localization, while internal dark regions highlight genomic regions containing riboproteome genes found to be most frequently amplified in the TCGA repository. (FIGS. 3D, 3K) Table detailing the top amplified genes as identified in the TCGA repository, and organized according to genomic loci. (FIGS. 3E and 3F) Summary from the TCGA for riboproteome genes in the regions of 3q and 8q respectively, that are found to be frequently amplified in human cancer. Left panels show the number of patients harboring an alteration from the datasets analyzed. As lung squamous cell carcinoma for the 3q locus and breast invasive carcinoma for the 8q locus were found to show high levels of alteration in both cases, the right panels illustrate the types of alteration found in these patients (amplification, homozygous deletion or mutation). (FIG. 3J). Amplification of the 1q22 locus in various cancer types. (FIGS. 3L, 3M)3q26 and 1q22 amplification observed in patients from lung adenocarcinoma and breast invasive carcinoma cancer datasets.

FIGS. 4A-4N show that riboproteomics uncovers ribosome-associated protein. (FIG. 4A) Western blot analysis of total lysates and polysomal fractions from PC3 and Du145 H and L labeled cell lines. Western blots for MARCKS, RpL7a, Integrin β1, RpS6, RpS14 and β-actin are shown. (FIG. 4B) Western blot analysis of pooled polysomal fractions showing differential enrichment of phospho-MARCKS and MARCKS from ribosomes of Du145, PC3, PWR1E and RWPE1 cells (right panel) and PC3, PPC1, Du145 and PWR1 E cells (left panel). Ponceau S staining served as a loading control. (FIG. 4C) Western blot analysis from pooled polysomal fractions validating ribosome-associated proteins from ribosomes of PC3, PPC1, Du145, RWPE1 and PWR1 E cells. For this analysis, polyribosomes have been isolated from all cell lines and fractions have been pooled to obtain subunits (S, fractions #3-5, see Figure S1A), early light polysomes (L, fractions #6-8, see Figure S1A) and late heavy polysomes (H, fractions #9-11, see FIG. 1G). Western blots for Integrin β1, IGF2BP3, hnRNPC1/2, Calmodulin, Hsp27, Hsp60 and NPM are shown. Ponceau S staining served as a loading control. (FIG. 4D) PC3 prostate cancer cells were subjected to puromycin-mediated dissociation of ribosome-mRNA complexes to demonstrate a specific association of MARCKS with the ribosome. Protein was isolated from individual fractions (#1-#7) of the sucrose gradients using TCA/DOC precipitation and subjected to western blot analysis for MARCKS. The relative distribution of MARCKS across the sucrose gradient was quantified using the Image J software (http://rsbweb.nih.gov/ij). (FIG. 4E) Western blot analysis from protein isolated from individual fractions across the sucrose gradients of PPC1 cell lysates treated with DMSO or PP242. Shift in LARP1 (upper left panels) and RpL4 (lower left panels) proteins can be readily observed upon treatment with the mTOR kinase inhibitor. Right panel: Rate of change between DMSO and PP242 conditions for LARP1 (light shading) and RpL4 (dark shading) from late to early fractions (#9-#3). FIGS. 4F, 4G). Protein enrichment on the polyribosomes of either normal or cancer cells. (FIG. 4H). Quantification of proteins associated with polyribosomes of prostate cancer cells. (FIG. 4I). Inhibition of mTOR by PP242 identifies a number of proteins that show a rapid and significant disassociation from the riboproteome upon treatment with PP242. (FIG. 4J). Western blot analysis of pooled polysomal fractions after puromycin treatment showing loss of RpS6 and RpL13a from polyribosomes. (FIG. 4K). Treatment of MN results in LARP1 dissociation with riboproteome components. (FIG. 4L). mTOR inhibition can selectively influence binding of LARP1 at the polysome. (FIG. 4M). Proteins that show differential association with polysomes from Npm1 wild type and null MEFs. (FIG. 4N). Ribonucleoprotein hnRNPC identified to be increased in polysomes of Npm1 null MEFs.

### DETAILED DESCRIPTION

There is a pressing need to understand in greater detail the many factors that contribute to ribosome function and the regulation of translation on a global scale. The inventors have analyzed in a non-biased, high-throughput manner the numerous factors that coordinate ribosome function and mRNA translation in several different cell lines and different cellular contexts.

A SILAC (stable isotope labeling by amino acids in cell culture)-based mass spectrometry approach was applied to comprehensively characterize the proteins that constitute the actively translating ribosome, i.e. the riboproteome, as defined by the proteins associated with (i) the ribosome itself, and which may be required for either directing translation or quality control of nascent proteins, and (ii) by the proteins associated with mRNAs undergoing active translation.

By employing this high throughput approach to the analysis of proteins associated with actively translating polysomes in various cellular populations and under varying conditions, a comprehensive overview of the riboproteome was obtained. Differential riboproteome components were identified amongst cancer cell lines and in the analysis of genetic and pharmacological perturbations to the riboproteome. A detailed characterization of the prostate riboproteome was presented, and the diversity of proteins that are associated with actively translating polysomes was highlighted. The data identify a number of novel components of the riboproteome, and demonstrate the ability of this approach to address the dynamic nature of the riboproteome upon specific perturbations.

The findings draw a number of important conclusions relating to the various elements that make up the riboproteome.

First, by cross referencing data from independent SILAC riboproteomic experiments, and using a comprehensive panel of prostate cell lines, a core group of proteins that are consistently identified in all experimental datasets was identified, while at least 70% of proteins quantified were found in at least 2 experimental datasets. From this global analysis, the dataset was shown to be highly enriched in factors that relate directly to the ribosome, to translational initiation and elongation, and to pathways that are known to regulate and control translation. Importantly, this comprehensive analysis also reveals that the riboproteome consists of significant proportion of RBPs. As recently reported, the mRNA-interactome revealed that a wide variety of proteins previously unappreciated as RBPs, can bind to mRNA (Castello et al., Cell 149:1393-1406, 2012; Baltz et al., Mol Cell 46:674-690, 2012). This diversity of RBP functionality is also observed in those RBPs represented in the dataset. However, there is also a large proportion of proteins that were identified, even as core riboproteome components, that are not annotated as having RNA binding properties, indicating a further layer of functional complexity in those proteins that work to regulate ribosome function and translation.

Second, the datasets indicate that the diversity within the riboproteome itself may have the capacity to categorize cell types and tissues and, importantly, may specifically contribute to regulation of gene expression within a given cellular compartment. Surprisingly, in the datasets that were analyzed, we the plasticity of the riboproteome does not appear to extend to individual ribosomal proteins themselves that are evenly represented in the various cell types investigated, and they appear to be uniformly altered in response to conditions that impact ribosomal translation, such as mTOR inhibition.

Third, further unexpected observations have been made by examining globally how the riboproteome may be altered in diseases such as human cancer. It was found that riboproteomic components display frequent copy number amplifications in human cancer, while genomic losses within the riboproteome is significantly less than that for non-riboproteomic genes. It was identified that three genomic loci around 3q26, 8q24, and 1q22 containing genes that appear to be altered in a significant number of patients for several of the cancer subtypes contained in the cBio TCGA database. It is worth noting that both the regions 3q26 and 8q24 contain the oncogenes PIK3CA and MYC respectively. While MYC and PIK3CA are frequently amplified in cancer, there are several examples within the cBio datasets that the riboproteomic genes within these regions may be amplified without co-amplification of the resident oncogene. It is also interesting to note that both MYC and the PI3-kinase signaling pathway represent important regulators of translation themselves.

Fourth, in addition to characterizing the riboproteome landscape in various cell types, proteins previously not known to be associated with actively translating ribosomes (e.g. MARCKS, Integrin β1 and IGF2BP3) was identified and validated. These proteins represent a number of interesting ribosome interactors and highlight the diversity of proteins that actually participate in translation. In addition, they also point to the potential of these datasets to identify novel regulators of translation.

Lastly, the data demonstrate that the cancer riboproteome can be pharmacologically modulated for therapy on the basis of this new molecular knowledge. On the one hand, the riboproteome responds dynamically and differentially to cancer drugs (e.g. rapamycin versus PP242), while, on the other hand it's differential composition could be used to tailor therapies and predict outcomes based on the riboproteomic profile of specific cell types.

Thus, quantitative, high throughput riboproteomics represents a powerful platform that can be readily applied to various cellular models to uncover how riboproteome composition contributes to organismal function and disease.

### Targets

A target can be identified as a protein that shows quantitative differences (e.g., change in gene expression, protein expression, or activity) between normal cells and cancer cell types. In another embodiment, a target can also be identified by comparing the quantitative differences between particular cancer cell types (e.g., cancer cell types that harbor distinct genetic alterations) to identify targets that are specific to certain cancer types. In certain aspects, the change in gene expression, protein expression, or activity may be a decrease, if for example, the target is an oncogenic protein (e.g., a decrease of about 2-fold, 3-fold, 4-fold, 5-fold, 8-fold, 10-fold) as compared to a normal reference (i.e., normal cells, or non-cancerous cells). In other aspects, the change in gene expression, protein expression, or activity may be an increase if, for example, the target is an antitumor target (e.g., an increase of about 2-fold, 3-fold, 4-fold, 5-fold, 8-fold, 10-fold) as compared to a normal reference.

In another embodiment, a target can be a protein that interacts with either the ribosome itself, the mRNAs that are being actively translated, or interact with other proteins that may have the capacity to interact with both the ribosome and mRNA.

Exemplary targets include, but is not limited to, those listed in Table 1 and Table 2.

TABLE 1
Targets Associated with the Components of the Riboproteome

| Number | IPI.Prot | GeneSymbol | DAVID.Official |
|---|---|---|---|
| 1 | IPI00395627 | CACYBP | CACYBP |
| 2 | IPI00550523 | ATL3 | ATL3 |
| 3 | IPI00794659 | RPS20 | rps20 |
| 4 | IPI00796337 | hCG_2017557 | PCBP2 |
| 5 | IPI00034049 | KIAA0221 | UPF1 |
| 6 | IPI00953461 | KIAA0217 | LARP4B |
| 7 | IPI00021263 | YWHAZ | YWHAZ |
| 8 | IPI00003881 | HNRNPF | HNRNPF |
| 9 | IPI00465431 | LGALS3 | LGALS3 |
| 10 | IPI00297982 | EIF2G | eif2s3 |
| 11 | IPI00005969 | CAPZA1 | CAPZA1 |
| 12 | IPI00018873 | NAMPT | NAMPT |
| 13 | IPI00019472 | ASCT2 | SLC1A5 |
| 14 | IPI00295992 | ATAD3A | atad3a |
| 15 | IPI00009841 | EWSR1 | LOC284685 |
| 16 | IPI00000581 | DKFZp564E242 | OTUB1 |
| 17 | IPI00013297 | HASPP28 | LOC645181 |
| 18 | IPI00025084 | CAPN4 | capns1 |
| 19 | IPI00172656 | ETEA | FAF2 |
| 20 | IPI00554723 | DXS648E | RPL10P15 |
| 21 | IPI00304925 | HSPA1 | HSPA1A |
| 22 | IPI00386755 | ERO1L | ERO1L |
| 23 | IPI00985019 | RPL28 | |
| 24 | IPI00023640 | PDCD5 | PDCD5 |
| 25 | IPI00418471 | VIM | VIM |
| 26 | IPI00003438 | DNAJC8 | DNAJC8 |
| 27 | IPI00414717 | CFR1 | GLG1 |
| 28 | IPI00021828 | CST6 | Cstb |
| 29 | IPI00844578 | DDX9 | dhx9 |
| 30 | IPI00749113 | DUT | dut |
| 31 | IPI00607584 | MYBBP1A | mybbp1a |
| 32 | IPI00021766 | KIAA0886 | rtn4 |
| 33 | IPI00410177 | ENSA | ENSA |
| 34 | IPI00220487 | ATP5H | atp5h |
| 35 | IPI00007927 | CAPE | SMC2 |
| 36 | IPI00300341 | TCEB1 | LOC100132973 |
| 37 | IPI00746655 | ESYT1 | Esyt1 |
| 38 | IPI00017963 | SNRPD1 | LOC119358 |
| 39 | IPI00007940 | C10orf69 | ERLIN1 |
| 40 | IPI00013744 | CD49B | ITGA2 |
| 41 | IPI00219037 | H2AFX | H2afx |
| 42 | IPI00000051 | PFD1 | PFDN1 |
| 43 | IPI00220271 | AKR1A1 | AKR1A1 |
| 44 | IPI00056494 | RPL36AL | RPL36AL |
| 45 | IPI00220871 | RPL37 | RPL37 |
| 46 | IPI00007764 | ARM2 | HN1 |
| 47 | IPI00921118 | ACTN1 | actn1 |
| 48 | IPI00910593 | CNN2 | CNN2 |
| 49 | IPI00299024 | BASP1 | Basp1 |
| 50 | IPI00914566 | FDPS | FDPS |
| 51 | IPI00854642 | KIAA0648 | PDS5A |
| 52 | IPI00024095 | ANX3 | anxa3 |
| 53 | IPI00005634 | KIAA0372 | TTC37 |
| 54 | IPI00157790 | ECM29 | KIAA0368 |
| 55 | IPI00026824 | HMOX2 | Hmox2 |
| 56 | IPI00026670 | TCEB2 | Tceb2 |
| 57 | IPI00219162 | RPL39 | RPL39P20 |
| 58 | IPI00646762 | NUDT5 | NUDT5 |
| 59 | IPI00456429 | UBA52 | uba52 |
| 60 | IPI00465132 | COPE | cope |
| 61 | IPI00289876 | STX7 | STX7 |
| 62 | IPI00026942 | C8orf2 | Erlin2 |
| 63 | IPI00011916 | AIMP2 | STAG3L3 |
| 64 | IPI00299524 | CAPD2 | NCAPD2 |
| 65 | IPI00032830 | CGI-114 | Rexo2 |
| 66 | IPI00010720 | CCT5 | cct5 |
| 67 | IPI00556451 | ETFB | etfB |
| 68 | IPI00938079 | DRIP4 | PDCD6IP |
| 69 | IPI00719752 | EIF3B | EIF3B |
| 70 | IPI00002214 | KPNA2 | KPNA2 |
| 71 | IPI00329692 | NMT | NMT1 |
| 72 | IPI00011126 | PSMC1 | psmc1 |
| 73 | IPI00216951 | DARS | Dars |
| 74 | IPI00761160 | CAST | cast |
| 75 | IPI00219005 | FKBP4 | fkbp4 |
| 76 | IPI00295400 | IFI53 | wars |
| 77 | IPI00924788 | AADACL1 | NCEH1 |
| 78 | IPI00046828 | CCDC58 | Ccdc58 |
| 79 | IPI00219526 | PGM1 | pgm1 |
| 80 | IPI00014589 | CLTB | Cltb |
| 81 | IPI00006052 | HSPC231 | PFDN2 |
| 82 | IPI00375631 | G1P2 | ISG15 |
| 83 | IPI00305692 | TRP32 | txnl1 |
| 84 | IPI00328319 | RBAP48 | LOC642954 |
| 85 | IPI00008380 | PPP2CA | PPP2CA |
| 86 | IPI00983296 | ACLY variant protein | |
| 87 | IPI01013296 | FER1L3 | |
| 88 | IPI00015029 | P23 | Ptges3 |
| 89 | IPI00015911 | DLD | dld |
| 90 | IPI00013683 | TUBB3 | MC1R |
| 91 | IPI01010055 | ANX11 | |
| 92 | IPI00644386 | FUBP1 | FUBP1 |
| 93 | IPI00798071 | NAP1L4 | nap1l4 |
| 94 | IPI00100796 | C9orf83 | Chmp5 |
| 95 | IPI00016862 | GLUR | gsr |
| 96 | IPI00470649 | NCLN | ncln |
| 97 | IPI00295542 | NUC | NUCB1 |
| 98 | IPI00030131 | LAP2 | TMPO |
| 99 | IPI00024403 | CPN3 | CPNE3 |
| 100 | IPI00014938 | HCC1 | SARNP |
| 101 | IPI00069750 | FIR | PUF60 |
| 102 | IPI00031420 | UGDH | UGDH |
| 103 | IPI00646839 | EIF3CL | EIF3CL |
| 104 | IPI00013290 | HDGF2 | Hdgfrp2 |
| 105 | IPI00646350 | NAE1 | Nae1 |
| 106 | IPI00215998 | CD63 | CD63 |
| 107 | IPI00010706 | GSS | gss |
| 108 | IPI00218775 | AIG6 | FKBP5 |
| 109 | IPI00218343 | TUBA1C | TUBA1C |
| 110 | IPI00783097 | GARS | Gars |
| 111 | IPI00012268 | PSMD2 | PSMD2 |
| 112 | IPI00027350 | NKEFB | PRDX2 |
| 113 | IPI00306382 | C1orf3 | Scamp3 |
| 114 | IPI00026625 | KIAA0791 | NUP155 |
| 115 | IPI00300371 | KIAA0017 | sf3b3 |
| 116 | IPI00220739 | HPR6.6 | PGRMC1 |
| 117 | IPI00329633 | TARS | Tars |
| 118 | IPI00828189 | PCMT1 | pcmt1 |
| 119 | IPI00002966 | APG2 | HSPA4 |
| 120 | IPI00004860 | RARS | rars |
| 121 | IPI00021785 | COX5B | COX5B |
| 122 | IPI00396435 | DBP1 | DHX15 |
| 123 | IPI00220834 | G22P2 | XRCC5 |
| 124 | IPI00219097 | HMG2 | HMGB2 |
| 125 | IPI00017367 | hCG_39182 | rdx |
| 126 | IPI00007001 | CGI-113 | mrpl11 |
| 127 | IPI00658000 | IGF2BP3 | igf2bp3 |
| 128 | IPI00007074 | YARS | Yars |
| 129 | IPI00007188 | ANT2 | SLC25A5 |
| 130 | IPI00306369 | NSUN2 | nsun2 |
| 131 | IPI00009922 | C14orf156 | C14orf156 |
| 132 | IPI00298520 | ARCN1 | Arcn1 |
| 133 | IPI00215637 | DBX | DDX3X |
| 134 | IPI00916503 | BZAP45 | BZW1 |
| 135 | IPI00402183 | HNRPQ | SYNCRIP |
| 136 | IPI00383581 | G2AN | GANAB |
| 137 | IPI00166010 | AD-005 | cnot1 |
| 138 | IPI00030275 | HSP75 | Trap1 |
| 139 | IPI00062120 | AAG13 | S100a16 |
| 140 | IPI00006482 | ATP1A1 | ATP1A1 |
| 141 | IPI00012007 | AHCY | ahcY |
| 142 | IPI00027230 | GRP94 | Hsp90b1 |
| 143 | IPI00794900 | MTHFC | mthfd1 |
| 144 | IPI00156689 | VAT1 | vat1 |
| 145 | IPI00215901 | ADK2 | Ak2 |
| 146 | IPI00220219 | COPB2 | copb2 |
| 147 | IPI00217477 | HMG2A | HMGB3 |
| 148 | IPI00783982 | COPG | CopG |
| 149 | IPI00746777 | ADH5 | ADH5 |
| 150 | IPI00644712 | G22P1 | LOC389901 |
| 151 | IPI00006211 | UNQ484/PRO983 | vapB |
| 152 | IPI00884105 | LAMP1 | lamp1 |
| 153 | IPI00654777 | hCG_1784554 | LOC390282 |
| 154 | IPI00909484 | CDC42 | |
| 155 | IPI00026216 | NPEPPS | npepps |
| 156 | IPI00009790 | PFKF | pfkp |
| 157 | IPI00029079 | GMPS | GMPS |
| 158 | IPI00004534 | KIAA0361 | PFAS |
| 159 | IPI00295851 | COPB | COPB1 |
| 160 | IPI00291922 | PSMA5 | psma5 |
| 161 | IPI00024175 | HSPC | PSMA7 |
| 162 | IPI00031522 | HADH | HADHA |
| 163 | IPI00013847 | UQCRC1 | Uqcrc1 |
| 164 | IPI00219525 | PGD | PGD |
| 165 | IPI00930710 | hCG_40633 | SLC25A13 |
| 166 | IPI00296337 | HYRC | PRKDC |
| 167 | IPI00165261 | C14orf163 | SCFD1 |
| 168 | IPI00790305 | PCNP | |
| 169 | IPI00218782 | CAPZB | CAPZB |
| 170 | IPI00784366 | ADTB2 | AP2B1 |
| 171 | IPI00453473 | H4/A | Hist1h4c |
| 172 | IPI00796366 | MYL6 | myl6 |
| 173 | IPI00032003 | EDMD | emd |
| 174 | IPI00102069 | EIF3M | EIF3M |
| 175 | IPI00418497 | PRO1512 | timm50 |
| 176 | IPI00019755 | GSTO1 | GSTO1 |
| 177 | IPI00016179 | S100A13 | S100A13 |
| 178 | IPI00005719 | RAB1 | RAB1A |
| 179 | IPI00215965 | HNRNPA1 | LOC644037 |
| 180 | IPI00382452 | CHMP1 | Chmp1a |
| 181 | IPI00025273 | GART | GART |
| 182 | IPI00014587 | CLTA | CLTA |
| 183 | IPI00183695 | ANX2LG | S100A10 |
| 184 | IPI00401264 | ERP44 | ERP44 |
| 185 | IPI00514399 | RP11-422P24.3-002 | RPS27P13 |
| 186 | IPI00472442 | HC2 | PSMA1 |
| 187 | IPI00815642 | TMSB4X | TMSL1 |
| 188 | IPI00647286 | C9orf88 | Fam129b |
| 189 | IPI00026087 | BAF | LOC645870 |
| 190 | IPI00007755 | KIAA0118 | rab21 |
| 191 | IPI00022977 | CKB | CKB |
| 192 | IPI00218568 | DCOH | Pcbd1 |
| 193 | IPI01024911 | PROS27 | |
| 194 | IPI00027341 | AFCP | capG |
| 195 | IPI00945633 | SSR1 | SSR1 |
| 196 | IPI00005657 | HKE2 | pfdn6 |
| 197 | IPI00290461 | EIF3J | EIF3J |
| 198 | IPI00299155 | HC9 | Psma4 |
| 199 | IPI00219034 | NDUFA8 | Ndufa8 |
| 200 | IPI00026268 | GNB1 | GNB1 |
| 201 | IPI00032561 | CAB39 | cab39 |
| 202 | IPI00006378 | CCDC72 | |
| 203 | IPI00026964 | UQCRFS1 | uqcrfs1 |
| 204 | IPI00396563 | SAKS1 | Ubxn1 |
| 205 | IPI00023542 | GP25L2 | tmed9 |
| 206 | IPI00008485 | ACO1 | ACO1 |
| 207 | IPI00293126 | CG22 | TBCB |
| 208 | IPI00943181 | PSME2 | PSME2 |
| 209 | IPI00220344 | GIG15 | |
| 210 | IPI00013068 | EIF3E | Eif3e |
| 211 | IPI00029631 | ERH | ERH |
| 212 | IPI00292771 | NUMA | NUMA1 |
| 213 | IPI00009960 | HMP | IMMT |
| 214 | IPI00384265 | C9orf10 | FAM120A |
| 215 | IPI00477313 | HNRNPC | Hnrnpc |
| 216 | IPI00413778 | FKBP1 | FKBP1A |
| 217 | IPI00942869 | CTNND1 | CTNND1 |
| 218 | IPI00017964 | SNRPD3 | snrpd3 |
| 219 | IPI00219029 | GOT1 | GOT1 |
| 220 | IPI00420084 | BID | BID |
| 221 | IPI00011603 | PSMD3 | psmd3 |
| 222 | IPI00031109 | NDUFA12L | NDUFAF2 |
| 223 | IPI01022656 | DDX17 | |
| 224 | IPI00299977 | CGI-202 | Phpt1 |
| 225 | IPI00009225 | STX8 | Stx8 |
| 226 | IPI00554681 | NDUFA5 | NDUFA5 |
| 227 | IPI00009407 | DAD1 | DAD1 |
| 228 | IPI00217466 | H1F3 | Hist1h1d |
| 229 | IPI00011604 | GCSH | LOC654085 |
| 230 | IPI00301434 | BOLA2 | bolA2 |
| 231 | IPI01015908 | DCTN2 | |
| 232 | IPI00399089 | KIAA0081 | MESDC2 |
| 233 | IPI00947070 | RPL22L1 | RPL22L1 |
| 234 | IPI00290738 | CALM | PICALM |
| 235 | IPI00413641 | AKR1B1 | AKR1B1 |
| 236 | IPI00176903 | FKSG13 | PTRF |
| 237 | IPI00291783 | GEMIN5 | GEMIN5 |
| 238 | IPI00936125 | DIAP1 | DIAPH1 |
| 239 | IPI00032140 | CBP1 | SERPINH1 |
| 240 | IPI00385042 | CRFG | GTPBP4 |
| 241 | IPI00172594 | MAAT1 | mrpl28 |
| 242 | IPI00473136 | CTNNA1 | Ctnna1 |
| 243 | IPI00478410 | ATP5C | Atp5c1 |
| 244 | IPI00830136 | C1orf31 | C1orf31 |
| 245 | IPI00305092 | PYM | wibg |
| 246 | IPI00220528 | PBSCF | SNRPF |
| 247 | IPI00216682 | CNN3 | cnn3 |
| 248 | IPI00002535 | FKBP13 | Fkbp2 |
| 249 | IPI00009253 | NAPA | napA |
| 250 | IPI00000105 | LRP | MVP |
| 251 | IPI00883655 | DPYSL2 | Dpysl2 |
| 252 | IPI00295857 | COPA | copA |
| 253 | IPI00004406 | UP | upp1 |
| 254 | IPI00328905 | IQGAP3 | Iqgap3 |
| 255 | IPI00291607 | ITPR3 | ITPR3 |
| 256 | IPI00031397 | ACS3 | ACSL3 |
| 257 | IPI00215997 | CD9 | CD9 |
| 258 | IPI00924816 | MTPN | mtpn |
| 259 | IPI00010779 | TPM4 | TPM4 |
| 260 | IPI00292657 | LTB4DH | PTGR1 |
| 261 | IPI00456008 | ATP5A | Atp5j |
| 262 | IPI00014149 | KIAA0103 | ttc35 |
| 263 | IPI00411559 | CAPC | SMC4 |
| 264 | IPI00220014 | IDI1 | IDI1 |
| 265 | IPI00030911 | VAMP8 | Vamp8 |
| 266 | IPI00396015 | ACAC | ACACA |
| 267 | IPI00037448 | GLXR | grhpr |
| 268 | IPI00020944 | FDFT1 | FDFT1 |
| 269 | IPI00913848 | FERMT2 | Fermt2 |
| 270 | IPI00333215 | GTF2S | TCEA1P2 |
| 271 | IPI00871174 | AMY1 | MYCBP |
| 272 | IPI00215777 | OK/SW-cl.48 | SLC25A3 |
| 273 | IPI00797738 | COX6B | COX6B1 |
| 274 | IPI00027681 | NNMT | nnmt |
| 275 | IPI00024670 | C5orf18 | REEP5 |
| 276 | IPI00009236 | CAV | cav1 |
| 277 | IPI00219825 | GLBA | PSAP |
| 278 | IPI00011631 | ZW10 | ZW10 |
| 279 | IPI00554788 | CYK18 | KRT18P19 |
| 280 | IPI00001734 | PSA | C8orf62 |
| 281 | IPI00011284 | COMT | COMT |
| 282 | IPI00001589 | TIM13B | Timm13 |
| 283 | IPI00019869 | S100A2 | S100A2 |
| 284 | IPI00002134 | KIAA0072 | PSMD5 |
| 285 | IPI00004838 | CRK | crk |
| 286 | IPI00031410 | FRAP | MTOR |
| 287 | IPI00783781 | C7orf14 | NUP205 |
| 288 | IPI00009634 | CGI-44 | SQRDL |
| 289 | IPI00893715 | EPCAM | epcam |
| 290 | IPI00010346 | AGTBP | NLN |
| 291 | IPI00329696 | CGI-90 | Fam82b |
| 292 | IPI00016925 | C10 | C12orf57 |
| 293 | IPI00289758 | CANPL2 | CAPN2 |
| 294 | IPI00011635 | BCL2L13 | BCL2L13 |
| 295 | IPI00966114 | SMN | |
| 296 | IPI01025459 | HE1 | |
| 297 | IPI01021644 | RAB5C | |
| 298 | IPI00027442 | AARS | AARS |
| 299 | IPI00014238 | KARS | KARS |
| 300 | IPI00465315 | CYC | CYCS |
| 301 | IPI00550069 | PRI | rnh1 |
| 302 | IPI00003527 | NHERF | SLC9A3R1 |
| 303 | IPI00027223 | IDH1 | IDH1 |
| 304 | IPI00031526 | C19orf43 | c19orf43 |
| 305 | IPI00293434 | SRP14 | SRP14 |
| 306 | IPI00166865 | CDGSH2 | cisd2 |
| 307 | IPI00021347 | UBCE7 | ube2l3 |
| 308 | IPI00030320 | DDX6 | DDX6 |
| 309 | IPI00294536 | MAWD | strap |
| 310 | IPI00021728 | EIF2B | Eif2s2 |
| 311 | IPI00429191 | ERF1 | etf1 |
| 312 | IPI00005198 | ILF2 | ilf2 |
| 313 | IPI00514587 | RP11-352P4.2-004 | SARS |
| 314 | IPI00916847 | GTPBP9 | Ola1 |
| 315 | IPI00374657 | VAP33 | vapa |
| 316 | IPI00010080 | KIAA1101 | OXSR1 |
| 317 | IPI00009104 | CGI-46 | RUVBL2 |
| 318 | IPI00790739 | ACO2 | ACO2 |
| 319 | IPI00878984 | DDT | ddt |
| 320 | IPI00019407 | H105E3 | nsdhl |
| 321 | IPI00025019 | PSC5 | psmb1 |
| 322 | IPI00910781 | GPI | GPI |
| 323 | IPI00420108 | DLST | DLST |
| 324 | IPI00785113 | GCF2 | Lrrfip1 |
| 325 | IPI00009368 | SFXN1 | SFXN1 |
| 326 | IPI00017184 | CDABP0131 | EHD1 |
| 327 | IPI00008418 | DIABLO | Diablo |
| 328 | IPI00024719 | HAT1 | HAT1 |
| 329 | IPI00646556 | NDUFV2 | Ndufv2 |
| 330 | IPI00026516 | OXCT | oxct1 |
| 331 | IPI00783313 | PYGL | PYGL |
| 332 | IPI00550451 | PPP1A | Ppp1ca |
| 333 | IPI00219861 | ACP1 | acp1 |
| 334 | IPI00004358 | PYGB | pygb |
| 335 | IPI00418262 | ALDC | aldoc |
| 336 | IPI00220059 | NDUFB4 | LOC402175 |
| 337 | IPI00023001 | C3orf28 | Fam162a |
| 338 | IPI00032406 | CPR3 | Dnaja2 |
| 339 | IPI00397904 | KIAA0095 | NUP93 |
| 340 | IPI00926977 | PSMC6 | Psmc6 |
| 341 | IPI00103142 | NUDCD2 | NUDCD2 |
| 342 | IPI00005537 | MRPL12 | |
| 343 | IPI00939163 | HSP105 | HSPH1 |
| 344 | IPI00031820 | FARS | farsa |
| 345 | IPI00414384 | C9orf99 | hsdl2 |
| 346 | IPI00386119 | SF1 | Sf1 |
| 347 | IPI00025974 | C20orf178 | CHMP4B |
| 348 | IPI00003627 | ACTL6A | actl6a |
| 349 | IPI00012578 | KPNA4 | KPNA4 |
| 350 | IPI00006213 | PCM1 | pcm1 |
| 351 | IPI00375380 | PSMD13 | PSMD13 |
| 352 | IPI00015972 | COX6C | COX6C |
| 353 | IPI00555917 | PXN | PXN |
| 354 | IPI00956559 | EIF4G1 | |
| 355 | IPI00027175 | SRI | SRI |
| 356 | IPI00939707 | KIAA0664 | kiaa0664 |
| 357 | IPI00006658 | PIN4 | PIN4 |
| 358 | IPI00015953 | DDX21 | DDX21 |
| 359 | IPI00719040 | C1orf77 | c1orf77 |
| 360 | IPI00018120 | DAP3 | DAP3 |
| 361 | IPI00005038 | HRSP12 | Hrsp12 |
| 362 | IPI00000861 | LASP1 | LASP1 |
| 363 | IPI00332499 | NASP | NASP |
| 364 | IPI00255052 | LYRM3 | NDUFB9 |
| 365 | IPI00019600 | MMS2 | ube2v2 |
| 366 | IPI00185374 | PSMD12 | PSMD12 |
| 367 | IPI00640197 | RP11-29B2.2-002 | tpp2 |
| 368 | IPI00033143 | ARG134 | EIF3K |
| 369 | IPI00025318 | SH3BGRL | SH3BGRL |
| 370 | IPI00219381 | NDUFA2 | Ndufa2 |
| 371 | IPI00008164 | PEP | prep |
| 372 | IPI00419237 | LAP3 | LAP3 |
| 373 | IPI00921422 | CKAP5 | ckap5 |
| 374 | IPI00303954 | CYB5B | CYB5B |
| 375 | IPI00220416 | UQBP | LOC442454 |
| 376 | IPI00941534 | CDC10 | 41159 |
| 377 | IPI00005966 | DAP13 | NDUFA12 |
| 378 | IPI00299608 | PSMD1 | PSMD1 |
| 379 | IPI00024742 | UQCRQ | UQCRQ |
| 380 | IPI00063903 | HCVFTP2 | USMG5 |
| 381 | IPI00000335 | HINT2 | HINT2 |
| 382 | IPI00855767 | RAP1GDS1 | Rap1gds1 |
| 383 | IPI00016703 | DHCR24 | DHCR24 |
| 384 | IPI00790503 | MYH10 | myh10 |
| 385 | IPI00024781 | FAM2C | SERF2 |
| 386 | IPI00414289 | COPS1 | GPS1 |
| 387 | IPI00073779 | HDCMD11P | MRPS35 |
| 388 | IPI00022694 | MCB1 | PSMD4 |
| 389 | IPI00004839 | CRKL | CRKL |
| 390 | IPI00217553 | BMRP | mRpL41 |
| 391 | IPI00294242 | IMOGN38 | mRpS31 |
| 392 | IPI00022276 | HSPC007 | mRpS28 |
| 393 | IPI00216138 | SM22 | TAGLN |
| 394 | IPI00012535 | DNAJ2 | Dnaja1 |
| 395 | IPI00020510 | C10orf70 | CISD1 |
| 396 | IPI00005107 | NPC1 | npc1 |
| 397 | IPI00333763 | C14orf87 | glrx5 |
| 398 | IPI00001458 | KIAA0166 | KNTC1 |
| 399 | IPI00009901 | NTF2 | NUTF2 |
| 400 | IPI00010136 | CTBP2 | CTBP2 |
| 401 | IPI00747849 | ATP1B | Atp1b1 |
| 402 | IPI00220063 | NDUFS5 | Ndufs5 |
| 403 | IPI00178352 | ABPL | FLNC |
| 404 | IPI00017292 | CTNNB | CTNNB1 |
| 405 | IPI00022442 | NDUFAB1 | NDUFAB1 |
| 406 | IPI00935906 | DCTN1 | DCTN1 |
| 407 | IPI00294501 | D7SR | DHCR7 |
| 408 | IPI00031023 | FLIl | flil |
| 409 | IPI00297910 | GA733-1 | TACSTD2 |
| 410 | IPI00747764 | IPOA7 | KPNA6 |
| 411 | IPI00006863 | SPAG7 | SPAG7 |
| 412 | IPI00909064 | COASY | COASY |
| 413 | IPI00001541 | TIM9 | TIMM9 |
| 414 | IPI00025285 | ATP6G | ATP6V1G1 |
| 415 | IPI00002186 | ARFGEF2 | ARFGEF2 |
| 416 | IPI00743335 | MYO1C | Myo1C |
| 417 | IPI00298111 | SNX6 | snx6 |
| 418 | IPI00005087 | TMOD3 | tmod3 |
| 419 | IPI00025725 | NDUFB1 | NDUFB1 |
| 420 | IPI00294578 | TGM2 | tgm2 |
| 421 | IPI00219604 | MAP2K1 | MAP2K1 |
| 422 | IPI00022334 | OAT | OAT |
| 423 | IPI00026833 | ADSS | ADSS |
| 424 | IPI00291419 | ACAT2 | Acat2 |
| 425 | IPI00333913 | NAG | NBAS |
| 426 | IPI00016046 | C20orf52 | ROMO1 |
| 427 | IPI00909465 | CDA016 | Yjefn3 |
| 428 | IPI00397358 | MPS1 | RPS27P13 |
| 429 | IPI00029266 | SNRPE | LOC100130109 |
| 430 | IPI00001661 | CHC1 | SNHG3-RCC1 |
| 431 | IPI00742124 | KIAA0794 | UBXN7 |
| 432 | IPI00043598 | IKBIP | ikbip |
| 433 | IPI00220573 | MLCB | MYL12A |
| 434 | IPI00797136 | IKBIP | ikbip |
| 435 | IPI00010214 | S100A14 | S100A14 |
| 436 | IPI00061531 | MRPL53 | MRPL53 |
| 437 | IPI00059242 | PRO3113 | Syap1 |
| 438 | IPI00339384 | ARSDR1 | rdh11 |
| 439 | IPI00020495 | DC47 | mrps36 |
| 440 | IPI00296022 | UQCRH | Uqcrh |
| 441 | IPI00022277 | CCDC56 | Ccdc56 |
| 442 | IPI00027448 | ATP5L | atp5l |
| 443 | IPI00166483 | C17orf61 | C17orf61 |
| 444 | IPI00024920 | ATP5D | Atp5d |
| 445 | IPI00015077 | EIF1 | LOC730144 |
| 446 | IPI00166528 | KIAA1999 | RICTOR |
| 447 | IPI00101968 | CMAP | DBNL |
| 448 | IPI00964886 | DDP2 | |
| 449 | IPI00020827 | hCG_40237 | |
| 450 | IPI01021606 | BAF170 | |
| 451 | IPI01025410 | ACYP1 | |
| 452 | IPI00968228 | DC2 | |
| 453 | IPI00955022 | TJP2 | |
| 454 | IPI00472416 | HLAB | |
| 455 | IPI00554481 | | SLC3A2 |
| 456 | IPI00021570 | | EDF1 |
| 457 | IPI00008552 | | glrx3 |
| 458 | IPI00289491 | | Casc3 |
| 459 | IPI00465233 | | EIF3L |
| 460 | IPI00328748 | | manf |
| 461 | IPI01020803 | | |
| 462 | IPI00473014 | | DSTN |
| 463 | IPI00017469 | | spr |
| 464 | IPI00015602 | | TOMM70A |
| 465 | IPI00033494 | | MYL12B |
| 466 | IPI00017726 | | HSD17B10 |
| 467 | IPI00010141 | | POLE3 |
| 468 | IPI00016513 | | RAB10 |
| 469 | IPI00017510 | | COX2 |
| 470 | IPI00026689 | | Cdk1 |
| 471 | IPI00010414 | | PDLIM1 |
| 472 | IPI00061525 | | Gnpnat1 |
| 473 | IPI00022597 | | UBE2MP1 |
| 474 | IPI00008964 | | RAB1B |
| 475 | IPI00031169 | | RAB2A |
| 476 | IPI00219445 | | PSME3 |
| 477 | IPI00024919 | | prdx3 |
| 478 | IPI00003588 | | EEF1E1 |
| 479 | IPI00260715 | | fus |
| 480 | IPI00009032 | | ssb |
| 481 | IPI00028888 | | HNRNPD |
| 482 | IPI00894409 | | Rbm12 |
| 483 | IPI00006980 | | c14orf166 |
| 484 | IPI00179953 | | NASP |
| 485 | IPI00021370 | | UBE2K |
| 486 | IPI00028055 | | tmed10 |
| 487 | IPI00029264 | | cyc1 |
| 488 | IPI00450472 | | UBE2I |
| 489 | IPI00479262 | | Eif4g1 |
| 490 | IPI00420014 | | SNRNP200 |
| 491 | IPI00220031 | | PXN |
| 492 | IPI00012545 | | TGOLN2 |
| 493 | IPI00301311 | | set |
| 494 | IPI00015952 | | EIF4G2 |
| 495 | IPI00004962 | | Golim4 |
| 496 | IPI00170692 | | vapa |
| 497 | IPI00018235 | | pef1 |
| 498 | IPI00465432 | | nomo3 |
| 499 | IPI00470779 | | TXLNA |
| 500 | IPI00549672 | | PSMD13 |
| 501 | IPI00382733 | | Lrrfip1 |
| 502 | IPI00027497 | | GPI |
| 503 | IPI00026111 | | tmco1 |
| 504 | IPI00554521 | | FTHL3 |
| 505 | IPI00397645 | | mxra7 |
| 506 | IPI00974176 | | |
| 507 | IPI00020127 | | RPA1 |
| 508 | IPI00306516 | | Timm44 |
| 509 | IPI00032881 | | MRPS23 |
| 510 | IPI00449049 | | parp1 |
| 511 | IPI00024642 | | Ccdc47 |
| 512 | IPI00797384 | | Larp4 |
| 513 | IPI00295098 | | srprb |
| 514 | IPI00413344 | | Cfl2 |
| 515 | IPI00217223 | | paics |
| 516 | IPI00022202 | | SLC25A3 |
| 517 | IPI00412987 | | gmfb |
| 518 | IPI00641181 | | MARCKSL1 |
| 519 | IPI00017160 | | vta1 |
| 520 | IPI00015361 | | PFDN5 |
| 521 | IPI00030706 | | AHSA1 |
| 522 | IPI00024976 | | Tomm22 |
| 523 | IPI00646493 | | copA |
| 524 | IPI00216088 | | crabp2 |
| 525 | IPI00744851 | | LOC100130009 |
| 526 | IPI00871851 | | 41154 |
| 527 | IPI00024661 | | SEC24C |
| 528 | IPI00216592 | | Hnrnpc |
| 529 | IPI00024097 | | tes |
| 530 | IPI00247871 | | tcerg1 |
| 531 | IPI00007935 | | PDLIM5 |
| 532 | IPI00015842 | | RCN1 |
| 533 | IPI00013723 | | PIN1 |
| 534 | IPI00015102 | | Alcam |
| 535 | IPI00024871 | | Cbfb |
| 536 | IPI00019918 | | DDX19A |
| 537 | IPI00004968 | | prpf19 |
| 538 | IPI00010860 | | Psmd9 |
| 539 | IPI00926109 | | DHX30 |
| 540 | IPI00027415 | | DHX36 |
| 541 | IPI00008575 | | Khdrbs1 |
| 542 | IPI00844215 | | SPTAN1 |
| 543 | IPI00010800 | | NES |
| 544 | IPI00004669 | | GALNT2 |
| 545 | IPI00022145 | | Nucks1 |
| 546 | IPI00013002 | | UBE2C |
| 547 | IPI00021537 | | Ogfr |
| 548 | IPI00004416 | | CHMP2A |
| 549 | IPI00643554 | | Rgs10 |
| 550 | IPI00303292 | | KPNA1 |
| 551 | IPI00003348 | | gnb2 |
| 552 | IPI00456925 | | DBNL |
| 553 | IPI00964764 | | |
| 554 | IPI00977912 | | |
| 555 | IPI00945725 | | Eif2a |
| 556 | IPI00009680 | | mrpl44 |
| 557 | IPI00456359 | | ATXN2L |
| 558 | IPI00478657 | | Grsf1 |
| 559 | IPI00916112 | | BZW1 |
| 560 | IPI00018140 | | SYNCRIP |
| 561 | IPI00218695 | | CALD1 |
| 562 | IPI00793201 | | AIMP1 |
| 563 | IPI00644037 | | Tecr |
| 564 | IPI00002135 | | TACC3 |
| 565 | IPI00006034 | | CRIP2 |
| 566 | IPI00017342 | | RHOG |
| 567 | IPI00291893 | | DCUN1D1 |
| 568 | IPI00023974 | | Pttg1ip |
| 569 | IPI00295772 | | CYP51A1 |
| 570 | IPI00216975 | | TPM4 |
| 571 | IPI00448751 | | KIAA1598 |
| 572 | IPI00554742 | | API5L1 |
| 573 | IPI00954530 | | ociad1 |
| 574 | IPI01011108 | | |
| 575 | IPI01022179 | | |
| 576 | IPI00216230 | | TMPO |
| 577 | IPI00306280 | | DENR |
| 578 | IPI00022002 | | MRPS27 |
| 579 | IPI00022078 | | ndrg1 |
| 580 | IPI00012795 | | EIF3I |
| 581 | IPI00170935 | | Lrrc47 |
| 582 | IPI00293655 | | DDX1 |
| 583 | IPI00427330 | | SBDS |
| 584 | IPI00910513 | | GSPT1 |
| 585 | IPI00220152 | | bccip |
| 586 | IPI00640703 | | Xpo5 |
| 587 | IPI00016910 | | EIF3C |
| 588 | IPI00013808 | | actn4 |
| 589 | IPI00182757 | | KIAA1967 |
| 590 | IPI00024364 | | tnpo1 |
| 591 | IPI00009057 | | G3BP2 |
| 592 | IPI00479306 | | PSMB5 |
| 593 | IPI00329536 | | EEA1 |
| 594 | IPI00299095 | | snx2 |
| 595 | IPI00549189 | | THOP1 |
| 596 | IPI00954806 | | |
| 597 | IPI00297084 | | |
| 598 | IPI00554777 | | asnS |
| 599 | IPI00028004 | | PSMB3 |
| 600 | IPI00006167 | | Ppm1g |
| 601 | IPI00009315 | | ACBD3 |
| 602 | IPI00472498 | | |
| 603 | IPI00555597 | | ptrh2 |
| 604 | IPI00334159 | | vbp1 |
| 605 | IPI00219622 | | psmA2 |
| 606 | IPI00976779 | | |
| 607 | IPI00411706 | | esd |
| 608 | IPI00903226 | | HK1 |
| 609 | IPI00218493 | | HPRT1 |
| 610 | IPI00604664 | | NDUFS1 |
| 611 | IPI00024650 | | SLC16A1 |
| 612 | IPI00419249 | | Psma3 |
| 613 | IPI00015954 | | sar1a |
| 614 | IPI00056357 | | c19orf10 |
| 615 | IPI00008569 | | YKT6 |
| 616 | IPI00925046 | | QARS |
| 617 | IPI00011250 | | Uchl3 |
| 618 | IPI00013214 | | MCM3 |
| 619 | IPI00290142 | | CTPS |
| 620 | IPI00015148 | | Rap1b |
| 621 | IPI00478231 | | Rhoa |
| 622 | IPI00784614 | | 41161 |
| 623 | IPI00014898 | | LOC652460 |
| 624 | IPI00028481 | | RAB8A |
| 625 | IPI00555956 | | PSMB4 |
| 626 | IPI00746004 | | Rps27l |
| 627 | IPI00218466 | | SEC61A1 |
| 628 | IPI00020928 | | TFAM |
| 629 | IPI00442073 | | CSRP1 |
| 630 | IPI00386803 | | LASP1 |
| 631 | IPI00011876 | | mtaP |
| 632 | IPI00410215 | | bpnt1 |
| 633 | IPI00010349 | | AGPS |
| 634 | IPI00646954 | | Enah |
| 635 | IPI00440703 | | gstk1 |
| 636 | IPI00221226 | | ANXA6 |
| 637 | IPI00003419 | | LOC645086 |
| 638 | IPI00063827 | | ABHD14B |
| 639 | IPI00789101 | | Ptges3 |
| 640 | IPI00013195 | | MRPL49 |
| 641 | IPI00441473 | | prmt5 |
| 642 | IPI00414320 | | anxa11 |
| 643 | IPI00647635 | | GIGYF2 |
| 644 | IPI00386114 | | Sf1 |
| 645 | IPI00168388 | | SRP68 |
| 646 | IPI00007928 | | Prpf8 |
| 647 | IPI00018627 | | naa50 |
| 648 | IPI00376344 | | MYO1B |
| 649 | IPI00215918 | | ARF4 |
| 650 | IPI00872359 | | DCTN1 |
| 651 | IPI00291939 | | Smc1a |
| 652 | IPI00007321 | | Lypla1 |
| 653 | IPI00003327 | | Arl3 |
| 654 | IPI00294879 | | rangap1 |
| 655 | IPI00452731 | | Ndufa7 |
| 656 | IPI00398922 | | ppp1r14b |
| 657 | IPI00215920 | | Arf6 |
| 658 | IPI00642584 | | KIAA0090 |
| 659 | IPI00219420 | | smc3 |
| 660 | IPI00645446 | | |
| 661 | IPI00018768 | | TSN |
| 662 | IPI00641582 | | bag3 |
| 663 | IPI00414819 | | SKIV2L |
| 664 | IPI00394838 | | ACLY |
| 665 | IPI00004902 | | etfB |
| 666 | IPI00927647 | | por |
| 667 | IPI00005904 | | ddx20 |
| 668 | IPI00061108 | | mrrf |
| 669 | IPI00307092 | | KARS |
| 670 | IPI00910701 | | AARS |
| 671 | IPI00395694 | | TNPO3 |
| 672 | IPI00641924 | | MRPS9 |
| 673 | IPI00552587 | | gadd45gip1 |
| 674 | IPI00783656 | | Mrpl38 |
| 675 | IPI00856045 | | AHNAK2 |
| 676 | IPI00025277 | | pdcd6 |
| 677 | IPI00909773 | | ube2l3 |
| 678 | IPI00013933 | | dsp |
| 679 | IPI00430812 | | CNBP |
| 680 | IPI00328715 | | MTDH |
| 681 | IPI00941649 | | HNRNPR |
| 682 | IPI00006181 | | eif3d |
| 683 | IPI00028122 | | PSIP1 |
| 684 | IPI00894287 | | HDLBP |
| 685 | IPI00232533 | | EIF1AX |
| 686 | IPI00020508 | | Trmt1 |
| 687 | IPI00306043 | | YTHDF2 |
| 688 | IPI00375441 | | FUBP1 |
| 689 | IPI00101186 | | Rrp12 |
| 690 | IPI00165230 | | DAZAP1 |
| 691 | IPI00005162 | | ARPC3 |
| 692 | IPI00018274 | | egfr |
| 693 | IPI00334282 | | FAM3C |
| 694 | IPI00220194 | | slc2a1 |
| 695 | IPI00001676 | | Nploc4 |
| 696 | IPI00218465 | | plaa |
| 697 | IPI00642211 | | Rnpep |
| 698 | IPI00290198 | | IL18 |
| 699 | IPI00017381 | | RfC4 |
| 700 | IPI00219682 | | stom |
| 701 | IPI00386271 | | slc25a12 |
| 702 | IPI00397860 | | cyb5a |
| 703 | IPI00797038 | | pck2 |
| 704 | IPI00556027 | | BAG5 |
| 705 | IPI00028160 | | HMBS |
| 706 | IPI00001091 | | AFG3L2 |
| 707 | IPI00796379 | | B2M |
| 708 | IPI00012490 | | atp2b4 |
| 709 | IPI00025717 | | MTX2 |
| 710 | IPI00007682 | | Atp6v1a |
| 711 | IPI00027717 | | GEMIN4 |
| 712 | IPI00005129 | | SCAMP1 |
| 713 | IPI00395939 | | PITPNB |
| 714 | IPI00443909 | | Cnpy2 |
| 715 | IPI00011662 | | Spint2 |
| 716 | IPI00307200 | | SWAP70 |
| 717 | IPI00026850 | | tspO |
| 718 | IPI00014808 | | PAFAH1B3 |
| 719 | IPI00010438 | | snap23 |
| 720 | IPI00009111 | | TPBG |
| 721 | IPI00019329 | | dynll1 |
| 722 | IPI00027240 | | gng5 |
| 723 | IPI00893197 | | ppp2r4 |
| 724 | IPI00657752 | | CD81 |
| 725 | IPI00217519 | | RALA |
| 726 | IPI00550364 | | pgm2 |
| 727 | IPI00011285 | | CAPN1 |
| 728 | IPI00005737 | | SURF4 |
| 729 | IPI00303868 | | GYS1 |
| 730 | IPI00027422 | | ITGB4 |
| 731 | IPI00216984 | | Calml3 |
| 732 | IPI00793381 | | PSMD6 |
| 733 | IPI00008599 | | EBP |
| 734 | IPI00219147 | | CSDAP1 |
| 735 | IPI00184363 | | GLTPP1 |
| 736 | IPI00329596 | | TMX2 |
| 737 | IPI00784320 | | Fam83h |
| 738 | IPI00384497 | | ptplb |
| 739 | IPI00030820 | | MRPL47 |
| 740 | IPI00554711 | | JUP |
| 741 | IPI00000821 | | mrpl16 |
| 742 | IPI00641384 | | SEC16A |
| 743 | IPI00329036 | | MRPL50 |
| 744 | IPI00247439 | | SLK |
| 745 | IPI00242630 | | heatr2 |
| 746 | IPI00020472 | | Tmem111 |
| 747 | IPI00029048 | | TTLL12 |
| 748 | IPI00016608 | | Tmed2 |
| 749 | IPI00072534 | | UNC45A |
| 750 | IPI00100748 | | HSPBP1 |
| 751 | IPI00221232 | | GNG12 |
| 752 | IPI00008998 | | ptplad1 |
| 753 | IPI00855820 | | MON2 |
| 754 | IPI00329629 | | DNAJC7 |
| 755 | IPI00018311 | | NPTN |
| 756 | IPI00170972 | | C9orf64 |
| 757 | IPI00302740 | | RPS4Y1 |
| 758 | IPI00030767 | | C19orf33 |
| 759 | IPI00171856 | | Dohh |
| 760 | IPI00790064 | | |
| 761 | IPI00955780 | | |
| 762 | IPI00981914 | | |
| 763 | IPI00980045 | | |
| 764 | IPI00220300 | | |
| 765 | IPI00030357 | | |
| 766 | IPI01021243 | | |
| 767 | IPI00298406 | | hadh |
| 768 | IPI00107753 | | Opa1 |
| 769 | IPI00005158 | | lonp1 |
| 770 | IPI00643591 | | Ap1g1 |
| 771 | IPI00007752 | | TUBB2C |
| 772 | IPI00013466 | | Asna1 |
| 773 | IPI00221224 | | ANPEP |
| 774 | IPI00032064 | | akap2 |
| 775 | IPI00029046 | | Mlec |
| 776 | IPI00644008 | | ELAVL2 |
| 777 | IPI00444452 | | MOV10 |
| 778 | IPI00456363 | | ATXN2L |
| 779 | IPI00456969 | | DYNC1H1 |
| 780 | IPI00221106 | | SF3B2 |
| 781 | IPI00646917 | | NUDT21 |
| 782 | IPI00418313 | | ilf3 |
| 783 | IPI00217661 | | RAVER1 |
| 784 | IPI00025815 | | TARDBP |
| 785 | IPI00005154 | | ssrp1 |
| 786 | IPI00290460 | | eif3g |
| 787 | IPI00029629 | | TRIM25 |
| 788 | IPI00844264 | | CSDE1 |
| 789 | IPI00022228 | | HDLBP |
| 790 | IPI00008982 | | ALDH18A1 |
| 791 | IPI00073713 | | MSI2 |
| 792 | IPI00334587 | | Hnrnpab |
| 793 | IPI00023334 | | mRpL4 |
| 794 | IPI00026519 | | PPIF |
| 795 | IPI00003927 | | ppiD |
| 796 | IPI00021435 | | PSMC2 |
| 797 | IPI00008868 | | MAP1B |
| 798 | IPI00304409 | | carhsp1 |
| 799 | IPI00299594 | | NRP1 |
| 800 | IPI00018783 | | itpa |
| 801 | IPI00024913 | | C21orf33 |
| 802 | IPI00006451 | | Nsf |
| 803 | IPI00299402 | | Pc |
| 804 | IPI00291136 | | COL6A1 |
| 805 | IPI00021267 | | EPHA2 |
| 806 | IPI00017375 | | SEC23A |
| 807 | IPI00296053 | | FH |
| 808 | IPI00641743 | | Hcfc1 |
| 809 | IPI00017303 | | Msh2 |
| 810 | IPI00394758 | | aldh3a2 |
| 811 | IPI00218319 | | TPM3 |
| 812 | IPI00011118 | | rrm2 |
| 813 | IPI00413451 | | serpinb6 |
| 814 | IPI00219025 | | GLRX |
| 815 | IPI00024307 | | efnb1 |
| 816 | IPI00025239 | | ndufs2 |
| 817 | IPI00021338 | | dlaT |
| 818 | IPI00030781 | | STAT1 |
| 819 | IPI00030363 | | ACAT1 |
| 820 | IPI00306290 | | Xpot |
| 821 | IPI00021440 | | ACTG1 |
| 822 | IPI00335385 | | DCPS |
| 823 | IPI00155601 | | macrod1 |
| 824 | IPI00941810 | | nomo3 |
| 825 | IPI00168479 | | APOA1BP |
| 826 | IPI00303568 | | PTGES2 |
| 827 | IPI00292953 | | RAI14 |
| 828 | IPI00147874 | | NANS |
| 829 | IPI00219729 | | Slc25a11 |
| 830 | IPI00003968 | | NDUFA9 |
| 831 | IPI00305978 | | AKR7A2 |
| 832 | IPI00012837 | | Kif5b |
| 833 | IPI00219675 | | rac1 |
| 834 | IPI00023919 | | PSMC5 |
| 835 | IPI00024664 | | USP5 |
| 836 | IPI00304577 | | AP2A1 |
| 837 | IPI00000811 | | PSMB6 |
| 838 | IPI00008219 | | RAD23A |
| 839 | IPI00293464 | | DDB1 |
| 840 | IPI00873355 | | ANXA10 |
| 841 | IPI00002441 | | sdc1 |
| 842 | IPI00292020 | | SRM |
| 843 | IPI00217943 | | LOC150786 |
| 844 | IPI00023510 | | rab5a |
| 845 | IPI00783625 | | SERPINB5 |
| 846 | IPI00291510 | | lmpdh2 |
| 847 | IPI00926935 | | gnai2 |
| 848 | IPI00027444 | | SERPINB1 |
| 849 | IPI00794221 | | DBN1 |
| 850 | IPI00384456 | | msh6 |
| 851 | IPI00553177 | | SERPINA1 |
| 852 | IPI00646899 | | RPL10P15 |
| 853 | IPI00102864 | | HK2P1 |
| 854 | IPI00016568 | | AK3L2 |
| 855 | IPI00009659 | | RPRD1B |
| 856 | IPI00031564 | | Ggct |
| 857 | IPI00007144 | | RPL26L1 |
| 858 | IPI00019148 | | C14orf19 |
| 859 | IPI00290039 | | CDCP1 |
| 860 | IPI00031131 | | C20orf3 |
| 861 | IPI00054042 | | LOC100093631 |
| 862 | IPI00010154 | | GDI1 |
| 863 | IPI00895800 | | INF2 |
| 864 | IPI00604773 | | PODXL |
| 865 | IPI00000690 | | AIFM1 |
| 866 | IPI00014361 | | TSTA3 |
| 867 | IPI00014577 | | RAB18 |
| 868 | IPI00514501 | | C1orf57 |
| 869 | IPI00549467 | | NIT2 |
| 870 | IPI00007102 | | GLOD4 |
| 871 | IPI00005578 | | Ehd4 |
| 872 | IPI00023647 | | UBA6 |
| 873 | IPI00337494 | | slc25a24 |
| 874 | IPI00383046 | | Cmbl |
| 875 | IPI00006952 | | LACTB2 |
| 876 | IPI00955965 | | |
| 877 | IPI00892541 | | |
| 878 | IPI01025218 | | |
| 879 | IPI00975549 | | |
| 880 | IPI00550308 | | |
| 881 | IPI01022651 | | |
| 882 | IPI00985090 | | |
| 883 | IPI01013419 | | |
| 884 | IPI01009057 | | |
| 885 | IPI01021966 | | |
| 886 | IPI00013508 | | actn1 |
| 887 | IPI00216260 | | TSFM |
| 888 | IPI00908931 | | PDCD5 |
| 889 | IPI00744648 | | Spag9 |
| 890 | IPI00748244 | | Spag9 |
| 891 | IPI00783862 | | BLVRB |
| 892 | IPI00152692 | | HARS2 |
| 893 | IPI00003482 | | DECR1 |
| 894 | IPI00003269 | | Actbl2 |
| 895 | IPI00328170 | | mogs |
| 896 | IPI00075607 | | fam192a |
| 897 | IPI00019903 | | TACO1 |
| 898 | IPI00000948 | | tbl2 |
| 899 | IPI00554469 | | IMMT |
| 900 | IPI00022793 | | hadhb |
| 901 | IPI00895806 | | CNBP |
| 902 | IPI00945964 | | GFM1 |
| 903 | IPI00031583 | | USO1 |
| 904 | IPI00026496 | | NPM3 |
| 905 | IPI00299254 | | Eif5b |
| 906 | IPI00298289 | | rtn4 |
| 907 | IPI00641579 | | Cirbp |
| 908 | IPI00942186 | | AKAP1 |
| 909 | IPI00784161 | | SUPT6H |
| 910 | IPI00021327 | | GRB2 |
| 911 | IPI00945864 | | FXR1 |
| 912 | IPI00060627 | | CCDC124 |
| 913 | IPI00641948 | | FUBP1 |
| 914 | IPI00011937 | | Prdx4 |
| 915 | IPI00016346 | | PROSC |
| 916 | IPI00550181 | | Chmp2b |
| 917 | IPI00025244 | | ZNF259 |
| 918 | IPI00384028 | | Papola |
| 919 | IPI00022314 | | Sod2 |
| 920 | IPI00017895 | | Gpd2 |
| 921 | IPI00294158 | | BLVRA |
| 922 | IPI00514424 | | PPT1 |
| 923 | IPI00021831 | | PRKAR1A |
| 924 | IPI00464979 | | SUCLA2 |
| 925 | IPI00012828 | | Acaa1 |
| 926 | IPI00027180 | | zmpste24 |
| 927 | IPI00032831 | | Snap29 |
| 928 | IPI00026663 | | ALDH1A3 |
| 929 | IPI00018272 | | Pnpo |
| 930 | IPI00216172 | | lamp2 |
| 931 | IPI00019912 | | hsd17b4 |
| 932 | IPI00019383 | | Galk1 |
| 933 | IPI00152900 | | Lzic |
| 934 | IPI00218924 | | Chp |
| 935 | IPI00925804 | | aip |
| 936 | IPI00011307 | | mthfd2 |
| 937 | IPI00220342 | | DDAH1 |
| 938 | IPI00015947 | | DNAJB1 |
| 939 | IPI00743293 | | RTN3 |
| 940 | IPI00096066 | | LOC283398 |
| 941 | IPI00003565 | | PSMD10 |
| 942 | IPI00010415 | | ACOT7 |
| 943 | IPI00019169 | | SH3GL1 |
| 944 | IPI00396630 | | PRKACA |
| 945 | IPI00010157 | | mat2a |
| 946 | IPI00479722 | | PSME1 |
| 947 | IPI00300567 | | dci |
| 948 | IPI00008986 | | SLC7A5 |
| 949 | IPI00013871 | | Rrm1 |
| 950 | IPI00017344 | | rab5b |
| 951 | IPI00910544 | | SERPINB5 |
| 952 | IPI00246975 | | GSTM3 |
| 953 | IPI00000792 | | cryz |
| 954 | IPI00455473 | | MIA3 |
| 955 | IPI00033022 | | DNM2 |
| 956 | IPI00300299 | | SPCS3 |
| 957 | IPI00005948 | | mri1 |
| 958 | IPI00026970 | | SUPT16HP |
| 959 | IPI00064193 | | TMX3 |
| 960 | IPI00549389 | | mettl11a |
| 961 | IPI00063130 | | TMEM205 |
| 962 | IPI00020530 | | ACOT13 |
| 963 | IPI00867509 | | CORO1C |
| 964 | IPI00332371 | | PFKL |
| 965 | IPI00019927 | | psmd7 |
| 966 | IPI00329600 | | sccpdh |
| 967 | IPI00009949 | | PSMF1 |
| 968 | IPI00020075 | | ABHD10 |
| 969 | IPI00867714 | | lsm12 |
| 970 | IPI00023234 | | UBA2 |
| 971 | IPI00032959 | | GPD1L |
| 972 | IPI00072377 | | set |
| 973 | IPI00065500 | | C1orf58 |
| 974 | IPI00796864 | | DPM1 |
| 975 | IPI00301518 | | MOBKL1B |
| 976 | IPI00003870 | | clpP |
| 977 | IPI00002525 | | NENF |
| 978 | IPI00166395 | | ACSF3 |
| 979 | IPI00384857 | | HN1 |
| 980 | IPI00513853 | | STX12 |
| 981 | IPI00927933 | | fam136a |
| 982 | IPI00017283 | | iars2 |
| 983 | IPI00410226 | | bolA2 |
| 984 | IPI00015897 | | LOC727896 |
| 985 | IPI00646240 | | HIST2H2BF |
| 986 | IPI01015321 | | |
| 987 | IPI01013834 | | |
| 988 | IPI00472151 | | |
| 989 | IPI00963908 | | |
| 990 | IPI01010281 | | |
| 991 | IPI00979324 | | |
| 992 | IPI00925052 | | |
| 993 | IPI01018120 | | |
| 994 | IPI00375531 | | |
| 995 | IPI00980337 | | |
| 996 | IPI00965726 | | |
| 997 | IPI00759776 | | actn1 |
| 998 | IPI00376317 | | EDC4 |
| 999 | IPI00782965 | | HIP1 |
| 1000 | IPI00179473 | | sqstm1 |
| 1001 | IPI00514983 | | HSPH1 |
| 1002 | IPI00304589 | | TNKS1BP1 |
| 1003 | IPI00300096 | | RAB35 |
| 1004 | IPI00221325 | | Ranbp2 |
| 1005 | IPI00019380 | | NCBP1 |
| 1006 | IPI00059292 | | MAGOHB |
| 1007 | IPI00217413 | | DHX29 |
| 1008 | IPI00167941 | | MDN1 |
| 1009 | IPI00016250 | | FXR2 |
| 1010 | IPI00182533 | | rpl28 |
| 1011 | IPI00895911 | | CNBP |
| 1012 | IPI00003519 | | EFTUD2 |
| 1013 | IPI00012493 | | rps20 |
| 1014 | IPI00100151 | | XRN2 |
| 1015 | IPI00022184 | | PUM2 |
| 1016 | IPI00000897 | | helz |
| 1017 | IPI00006987 | | DDX24 |
| 1018 | IPI00300789 | | STAU2 |
| 1019 | IPI00017451 | | Sf3a1 |
| 1020 | IPI00026089 | | sf3b1 |
| 1021 | IPI00003704 | | RBM4 |
| 1022 | IPI00291016 | | NDUFV3 |
| 1023 | IPI00216247 | | PSMD4 |
| 1024 | IPI00399170 | | UPF1 |
| 1025 | IPI00016249 | | FXR1 |
| 1026 | IPI00293331 | | POP1 |
| 1027 | IPI00220158 | | Add1 |
| 1028 | IPI00419373 | | Hnrnpa3 |
| 1029 | IPI00386189 | | naa15 |
| 1030 | IPI00179298 | | HUWE1 |
| 1031 | IPI00745433 | | EIF2C2 |
| 1032 | IPI00162330 | | MRPL37 |
| 1033 | IPI00783302 | | Ptcd3 |
| 1034 | IPI00217686 | | Ftsj3 |
| 1035 | IPI00396627 | | ELAC2 |
| 1036 | IPI00012066 | | PCBP2 |
| 1037 | IPI00006408 | | nosip |
| 1038 | IPI00005614 | | SPTBN1 |
| 1039 | IPI00893067 | | eml4 |
| 1040 | IPI00465294 | | Cdc5l |
| 1041 | IPI00927150 | | CHCHD3 |
| 1042 | IPI00553024 | | EYA4 |
| 1043 | IPI00297492 | | stt3a |
| 1044 | IPI00015973 | | EPB41L2 |
| 1045 | IPI00220578 | | GNAI3 |
| 1046 | IPI00926820 | | Slc4a7 |
| 1047 | IPI00012369 | | Mad2l1 |
| 1048 | IPI00289819 | | Igf2r |
| 1049 | IPI00513827 | | ACADM |
| 1050 | IPI00423570 | | Kras |
| 1051 | IPI00023004 | | EIF1AY |
| 1052 | IPI00027192 | | PLOD1 |
| 1053 | IPI00909772 | | AP2M1 |
| 1054 | IPI00031517 | | MCM6 |
| 1055 | IPI00152540 | | CD109 |
| 1056 | IPI00183054 | | MIB1 |
| 1057 | IPI00106506 | | Ecsit |
| 1058 | IPI00217354 | | ARFGAP1 |
| 1059 | IPI00220648 | | PMVK |
| 1060 | IPI00008436 | | Pole4 |
| 1061 | IPI00008034 | | Rab23 |
| 1062 | IPI00008943 | | DDX19B |
| 1063 | IPI00025178 | | BCAS2 |
| 1064 | IPI00470573 | | Actr2 |
| 1065 | IPI00021346 | | UBE2E1 |
| 1066 | IPI00018350 | | Mcm5 |
| 1067 | IPI00018349 | | MCM4 |
| 1068 | IPI00927614 | | golga4 |
| 1069 | IPI00219673 | | gstk1 |
| 1070 | IPI00028006 | | psmb2 |
| 1071 | IPI00945153 | | ndufa6 |
| 1072 | IPI00908444 | | CAMK2G |
| 1073 | IPI00010882 | | DFFA |
| 1074 | IPI00007019 | | ppil1 |
| 1075 | IPI00328257 | | AP1B1 |
| 1076 | IPI00926925 | | Ogdh |
| 1077 | IPI00889196 | | uqcrfs1 |
| 1078 | IPI00216057 | | sorD |
| 1079 | IPI00005179 | | Polr1c |
| 1080 | IPI00910088 | | ap1m1 |
| 1081 | IPI00922181 | | mcm2 |
| 1082 | IPI00016746 | | Cbfb |
| 1083 | IPI00093057 | | CPOX |
| 1084 | IPI00900325 | | Nup214 |
| 1085 | IPI00002459 | | ANXA6 |
| 1086 | IPI00020719 | | MAVS |
| 1087 | IPI00647457 | | HLA-A |
| 1088 | IPI00514550 | | DNM2 |
| 1089 | IPI00006072 | | sec61g |
| 1090 | IPI00009822 | | LOC650638 |
| 1091 | IPI00083708 | | Bat2l2 |
| 1092 | IPI00853161 | | RPL10P15 |
| 1093 | IPI00031801 | | CSDAP1 |
| 1094 | IPI00479743 | | POTEE |
| 1095 | IPI00855856 | | API5L1 |
| 1096 | IPI00554701 | | Uqcr10 |
| 1097 | IPI00171626 | | lpcat1 |
| 1098 | IPI00759658 | | MSMP |
| 1099 | IPI00042580 | | apoo |
| 1100 | IPI00007277 | | lrrfip2 |
| 1101 | IPI00647837 | | ZNF185 |
| 1102 | IPI00148063 | | HEBP1 |
| 1103 | IPI00911039 | | HSPA1A |
| 1104 | IPI00845339 | | HSPA1A |
| 1105 | IPI00171421 | | C8orf55 |
| 1106 | IPI00643435 | | atad3a |
| 1107 | IPI00465156 | | ADCY4 |
| 1108 | IPI00022827 | | SLK |
| 1109 | IPI00419802 | | HIBCH |
| 1110 | IPI00872556 | | Mobkl1a |
| 1111 | IPI00010204 | | SFRS3 |
| 1112 | IPI00018871 | | Arl8b |
| 1113 | IPI00217975 | | LMNB1 |
| 1114 | IPI00168640 | | C19orf18 |
| 1115 | IPI00909387 | | GHITM |
| 1116 | IPI00553153 | | Atpif1 |
| 1117 | IPI00007309 | | TIMM23B |
| 1118 | IPI00871988 | | SFXN3 |
| 1119 | IPI00555902 | | ociad2 |
| 1120 | IPI00478450 | | NDUFB11 |
| 1121 | IPI00982376 | | |
| 1122 | IPI00982951 | | |
| 1123 | IPI00736446 | | |
| 1124 | IPI00382926 | | |
| 1125 | IPI01018609 | | |
| 1126 | IPI00983078 | | |
| 1127 | IPI01019005 | | |
| 1128 | IPI00910816 | | |
| 1129 | IPI00967716 | | |
| 1130 | IPI00982101 | | |
| 1131 | IPI00953272 | | |
| 1132 | IPI01024808 | | |
| 1133 | IPI01012733 | | |
| 1134 | IPI00382990 | | |
| 1135 | IPI00386208 | | |
| 1136 | IPI00896727 | | CAND1 |
| 1137 | IPI00648173 | | CLTC |
| 1138 | IPI00123494 | | PSMD2 |
| 1139 | IPI00125901 | | rps13 |
| 1140 | IPI00315488 | | rars |
| 1141 | IPI00169463 | | TUBB2C |
| 1142 | IPI00108125 | | eif5a |
| 1143 | IPI00663627 | | FLNB |
| 1144 | IPI00929813 | | NAP1L1 |
| 1145 | IPI00224152 | | Apex1 |
| 1146 | IPI00122565 | | GDI2 |
| 1147 | IPI00331461 | | rpl11 |
| 1148 | IPI00133985 | | Ruvbl1 |
| 1149 | IPI00132250 | | Eif3e |
| 1150 | IPI00229859 | | EIF3B |
| 1151 | IPI00890117 | | CFL1 |
| 1152 | IPI00331556 | | HSPA4 |
| 1153 | IPI00117978 | | COX4I1 |
| 1154 | IPI00230395 | | ANXA1 |
| 1155 | IPI00113377 | | rplp1 |
| 1156 | IPI00116283 | | Cct3 |
| 1157 | IPI00131357 | | rps23 |
| 1158 | IPI00555059 | | Prdx6 |
| 1159 | IPI00457898 | | pgam1 |
| 1160 | IPI00342766 | | HP1BP3 |
| 1161 | IPI00454008 | | SHMT2 |
| 1162 | IPI00410937 | | RBM8A |
| 1163 | IPI00312018 | | Mlec |
| 1164 | IPI00776018 | | DYNC1I2 |
| 1165 | IPI00319973 | | PGRMC1 |
| 1166 | IPI00467833 | | TPI1 |
| 1167 | IPI00113870 | | pcnA |
| 1168 | IPI00129519 | | Basp1 |
| 1169 | IPI00163011 | | TXNDC5 |
| 1170 | IPI00467447 | | IQGAP1 |
| 1171 | IPI00117910 | | PRDX2 |
| 1172 | IPI00227299 | | VIM |
| 1173 | IPI00123802 | | HSPH1 |
| 1174 | IPI00944194 | | Fkbp10 |
| 1175 | IPI00132474 | | Itgb1 |
| 1176 | IPI00854971 | | pdia6 |
| 1177 | IPI00757312 | | myh10 |
| 1178 | IPI00269613 | | EIF3I |
| 1179 | IPI00469268 | | CCT8 |
| 1180 | IPI00620256 | | lmna |
| 1181 | IPI00222514 | | MRPS27 |
| 1182 | IPI00227808 | | CDV3 |
| 1183 | IPI00929758 | | TLN1 |
| 1184 | IPI00828412 | | RBBP4 |
| 1185 | IPI00551412 | | MAGOH |
| 1186 | IPI00116279 | | cct5 |
| 1187 | IPI00408495 | | bsg |
| 1188 | IPI00403589 | | ERH |
| 1189 | IPI00330862 | | EZR |
| 1190 | IPI00114733 | | SERPINH1 |
| 1191 | IPI00131459 | | Nme1 |
| 1192 | IPI00116277 | | cct4 |
| 1193 | IPI00757359 | | Caprin1 |
| 1194 | IPI00353563 | | fscn1 |
| 1195 | IPI00123379 | | HDLBP |
| 1196 | IPI00131056 | | IGF2BP1 |
| 1197 | IPI00762774 | | eif3d |
| 1198 | IPI00112448 | | rps10 |
| 1199 | IPI00130280 | | Atp5a1 |
| 1200 | IPI00222546 | | rpl22 |
| 1201 | IPI00135686 | | ppiB |
| 1202 | IPI00116498 | | YWHAZ |
| 1203 | IPI00229517 | | LGALS1 |
| 1204 | IPI00321884 | | NVL |
| 1205 | IPI00230133 | | Hist1h1b |
| 1206 | IPI00322749 | | SNRPD1 |
| 1207 | IPI00118384 | | YWHAE |
| 1208 | IPI00323806 | | rpl24 |
| 1209 | IPI00122743 | | Dars |
| 1210 | IPI00330804 | | HSP90AA1 |
| 1211 | IPI00626385 | | PLEC |
| 1212 | IPI00123313 | | Uba1 |
| 1213 | IPI00116302 | | Eif2s2 |
| 1214 | IPI00230108 | | PDIA3 |
| 1215 | IPI00123007 | | rpl31 |
| 1216 | IPI00627049 | | RpL35A |
| 1217 | IPI00108271 | | ELAVL1 |
| 1218 | IPI00137787 | | rpl8 |
| 1219 | IPI00408796 | | Sf3a1 |
| 1220 | IPI00830478 | | FAM120A |
| 1221 | IPI00323592 | | mdh2 |
| 1222 | IPI00317740 | | GNB2L1 |
| 1223 | IPI00224729 | | Hnrnph1 |
| 1224 | IPI00137730 | | PEBP1 |
| 1225 | IPI00113223 | | FASN |
| 1226 | IPI00270737 | | FMR1 |
| 1227 | IPI00123639 | | CALR |
| 1228 | IPI00407571 | | ssrp1 |
| 1229 | IPI00230707 | | YWHAG |
| 1230 | IPI00132950 | | rps21 |
| 1231 | IPI00118676 | | EIF4A1 |
| 1232 | IPI00128867 | | purb |
| 1233 | IPI00828223 | | SRP68 |
| 1234 | IPI00117689 | | PTRF |
| 1235 | IPI00133903 | | HSPA9 |
| 1236 | IPI00134599 | | rps3 |
| 1237 | IPI00396671 | | ABCF1 |
| 1238 | IPI00930882 | | SLC3A2 |
| 1239 | IPI00221581 | | EIF4B |
| 1240 | IPI00222461 | | gnl3 |
| 1241 | IPI00129276 | | eif3a |
| 1242 | IPI00108818 | | Gnl3l |
| 1243 | IPI00112963 | | Ctnna1 |
| 1244 | IPI00828741 | | RALY |
| 1245 | IPI00120691 | | DDX21 |
| 1246 | IPI00330599 | | MTDH |
| 1247 | IPI00309035 | | RPN1 |
| 1248 | IPI00271951 | | Pdia4 |
| 1249 | IPI00313475 | | Atp5c1 |
| 1250 | IPI00133522 | | p4hb |
| 1251 | IPI00123129 | | SND1 |
| 1252 | IPI00130883 | | RBM3 |
| 1253 | IPI00123281 | | LRRC59 |
| 1254 | IPI00119618 | | Canx |
| 1255 | IPI00420726 | | rps9 |
| 1256 | IPI00387422 | | ZYX |
| 1257 | IPI00128202 | | eIF3h |
| 1258 | IPI00229080 | | HSP90AB1 |
| 1259 | IPI00119876 | | DYNC1H1 |
| 1260 | IPI00123891 | | CSRP1 |
| 1261 | IPI00755226 | | SEC61B |
| 1262 | IPI00830528 | | MOV10 |
| 1263 | IPI00223047 | | Ckap4 |
| 1264 | IPI00133428 | | psmc1 |
| 1265 | IPI00406117 | | SYNCRIP |
| 1266 | IPI00111877 | | SSBP1 |
| 1267 | IPI00223713 | | Hist1h1c |
| 1268 | IPI00121514 | | stip1 |
| 1269 | IPI00463573 | | EIF3L |
| 1270 | IPI00119063 | | lrp1 |
| 1271 | IPI00116281 | | cct6a |
| 1272 | IPI00316133 | | SRP72 |
| 1273 | IPI00761863 | | lgf2bp2 |
| 1274 | IPI00944141 | | Rps5 |
| 1275 | IPI00762542 | | rps11 |
| 1276 | IPI00381291 | | PSMD4 |
| 1277 | IPI00331315 | | igf2bp3 |
| 1278 | IPI00124287 | | PABPC1 |
| 1279 | IPI00118899 | | actn4 |
| 1280 | IPI00111412 | | rpl4 |
| 1281 | IPI00162790 | | RpL18A |
| 1282 | IPI00377441 | | RPS26 |
| 1283 | IPI00420949 | | UPF1 |
| 1284 | IPI00130095 | | G3BP1 |
| 1285 | IPI00128904 | | Pcbp1 |
| 1286 | IPI00132443 | | hnrnpm |
| 1287 | IPI00230035 | | DDX3X |
| 1288 | IPI00554845 | | IMMT |
| 1289 | IPI00929786 | | LARP1 |
| 1290 | IPI00469392 | | rtn4 |
| 1291 | IPI00124742 | | eif4h |
| 1292 | IPI00109764 | | TOP1 |
| 1293 | IPI00320016 | | nonO |
| 1294 | IPI00127707 | | PCBP2 |
| 1295 | IPI00828620 | | Larp4 |
| 1296 | IPI00622371 | | eif3g |
| 1297 | IPI00921658 | | FLNA |
| 1298 | IPI00277066 | | Hnrnpab |
| 1299 | IPI00858249 | | Eif4g1 |
| 1300 | IPI00330591 | | csdA |
| 1301 | IPI00321647 | | EIF3C |
| 1302 | IPI00317794 | | ncl |
| 1303 | IPI00119305 | | PA2G4 |
| 1304 | IPI00126716 | | Eif4a3 |
| 1305 | IPI00330958 | | HNRNPD |
| 1306 | IPI00122559 | | Ktn1 |
| 1307 | IPI00134300 | | ssb |
| 1308 | IPI00471475 | | serbp1 |
| 1309 | IPI00121758 | | TARDBP |
| 1310 | IPI00458583 | | Hnrnpu |
| 1311 | IPI00331361 | | mybbp1a |
| 1312 | IPI00623284 | | sf3b1 |
| 1313 | IPI00553798 | | ahnak |
| 1314 | IPI00223443 | | Hnrnpc |
| 1315 | IPI00775950 | | CALD1 |
| 1316 | IPI00752108 | | CTNND1 |
| 1317 | IPI00648318 | | Ak2 |
| 1318 | IPI00122862 | | mthfd1 |
| 1319 | IPI00555113 | | RPL18 |
| 1320 | IPI00229534 | | MARCKS |
| 1321 | IPI00667117 | | DBI |
| 1322 | IPI00277001 | | Psma4 |
| 1323 | IPI00311682 | | ATP1A1 |
| 1324 | IPI00137735 | | RPS25 |
| 1325 | IPI00115097 | | copb2 |
| 1326 | IPI00170008 | | snrpa1 |
| 1327 | IPI00314736 | | ANP32A |
| 1328 | IPI00123181 | | MYH9 |
| 1329 | IPI00110588 | | msn |
| 1330 | IPI00319992 | | HSPA5 |
| 1331 | IPI00849793 | | rpl12 |
| 1332 | IPI00111831 | | Naca |
| 1333 | IPI00457499 | | gcn1l1 |
| 1334 | IPI00230440 | | ahcY |
| 1335 | IPI00125778 | | TAGLN2 |
| 1336 | IPI00119224 | | snrpd3 |
| 1337 | IPI00129526 | | Hsp90b1 |
| 1338 | IPI00468481 | | ATP5B |
| 1339 | IPI00653179 | | FKBP1A |
| 1340 | IPI00113536 | | anp32b |
| 1341 | IPI00112414 | | CSE1L |
| 1342 | IPI00133948 | | Fkbp2 |
| 1343 | IPI00137409 | | tkt |
| 1344 | IPI00230682 | | YWHAB |
| 1345 | IPI00223437 | | CopG |
| 1346 | IPI00111271 | | srprb |
| 1347 | IPI00125143 | | ARPC1B |
| 1348 | IPI00849113 | | Fau |
| 1349 | IPI00226515 | | TAGLN |
| 1350 | IPI00131845 | | Psma6 |
| 1351 | IPI00856379 | | ALDOA |
| 1352 | IPI00270326 | | PSMC2 |
| 1353 | IPI00132194 | | AIMP1 |
| 1354 | IPI00754096 | | clint1 |
| 1355 | IPI00323881 | | KPNB1 |
| 1356 | IPI00624653 | | USMG5 |
| 1357 | IPI00133243 | | ifitm3 |
| 1358 | IPI00127841 | | LOC631229 |
| 1359 | IPI00132050 | | LOC675851 |
| 1360 | IPI00120045 | | Gm2903 |
| 1361 | IPI00114162 | | Fabp5 |
| 1362 | IPI00474407 | | Gm8667 |
| 1363 | IPI00132390 | | Gm3244 |
| 1364 | IPI00874728 | | tpm2 |
| 1365 | IPI00775791 | | manf |
| 1366 | IPI00928004 | | copA |
| 1367 | IPI00828225 | | BCAP31 |
| 1368 | IPI00114209 | | glud1 |
| 1369 | IPI00222419 | | Gm10108 |
| 1370 | IPI00474446 | | Gm7459 |
| 1371 | IPI00230660 | | Gm14166 |
| 1372 | IPI00468203 | | LOC100048867 |
| 1373 | IPI00153103 | | AIMP2 |
| 1374 | IPI00469918 | | LOC100047501 |
| 1375 | IPI00310091 | | PPP2R1A |
| 1376 | IPI00130322 | | Ndufa7 |
| 1377 | IPI00113845 | | psmb1 |
| 1378 | IPI00133931 | | FKBP11 |
| 1379 | IPI00331174 | | Cct7 |
| 1380 | IPI00315100 | | Gm12844 |
| 1381 | IPI00121309 | | Gm12251 |
| 1382 | IPI00272545 | | Gm4149 |
| 1383 | IPI00116308 | | st13 |
| 1384 | IPI00318548 | | Gm6978 |
| 1385 | IPI00664670 | | FLNC |
| 1386 | IPI00308162 | | slc25a12 |
| 1387 | IPI00408215 | | MYO1B |
| 1388 | IPI00129323 | | Gm7083 |
| 1389 | IPI00126042 | | Rab14 |
| 1390 | IPI00122421 | | LOC631649 |
| 1391 | IPI00118963 | | mRpL12 |
| 1392 | IPI00281011 | | Marcksl1-ps3 |
| 1393 | IPI00115538 | | LOC100047211 |
| 1394 | IPI00405227 | | vcl |
| 1395 | IPI00323971 | | Gm15210 |
| 1396 | IPI00341282 | | Gm12231 |
| 1397 | IPI00320217 | | CCT2 |
| 1398 | IPI00929832 | | NUP205 |
| 1399 | IPI00111770 | | Gm2972 |
| 1400 | IPI00116120 | | Gm3837 |
| 1401 | IPI00136251 | | Dnaja2 |
| 1402 | IPI00114491 | | Cdk1 |
| 1403 | IPI00222548 | | Gm5561 |
| 1404 | IPI00123604 | | LOC676179 |
| 1405 | IPI00623776 | | Hist1h4c |
| 1406 | IPI00113257 | | Gm7181 |
| 1407 | IPI00119239 | | PSMB6 |
| 1408 | IPI00133234 | | tmem33 |
| 1409 | IPI00915054 | | Gm3379 |
| 1410 | IPI00224505 | | LOC674921 |
| 1411 | IPI00849927 | | Gm15451 |
| 1412 | IPI00111258 | | MVP |
| 1413 | IPI00127942 | | DSTN |
| 1414 | IPI00131771 | | Gm16399 |
| 1415 | IPI00263879 | | Gm4342 |
| 1416 | IPI00113895 | | ACTR1A |
| 1417 | IPI00269661 | | Gm5550 |
| 1418 | IPI00129178 | | OAT |
| 1419 | IPI00849044 | | LOC100044039 |
| 1420 | IPI00753815 | | Spna2 |
| 1421 | IPI00465880 | | Gm14586 |
| 1422 | IPI00129577 | | AIFM1 |
| 1423 | IPI00224219 | | SCFD1 |
| 1424 | IPI00459493 | | TCP1 |
| 1425 | IPI00606508 | | Gm9168 |
| 1426 | IPI00338964 | | ATP2A2 |
| 1427 | IPI00465568 | | Gm11878 |
| 1428 | IPI00227835 | | TPM1 |
| 1429 | IPI00221613 | | Gm8230 |
| 1430 | IPI00125460 | | LOC674583 |
| 1431 | IPI00117352 | | Tubb5 |
| 1432 | IPI00420261 | | LOC674543 |
| 1433 | IPI00620145 | | LOC631033 |
| 1434 | IPI00224784 | | Gm7614 |
| 1435 | IPI00120716 | | GNB1 |
| 1436 | IPI00944143 | | Serpinb6a |
| 1437 | IPI00230507 | | LOC674469 |
| 1438 | IPI00113660 | | PSME3 |
| 1439 | IPI00118986 | | Gm5436 |
| 1440 | IPI00404551 | | CTSD |
| 1441 | IPI00225634 | | Gm6646 |
| 1442 | IPI00283862 | | PSMA1 |
| 1443 | IPI00109611 | | Fam162a |
| 1444 | IPI00117705 | | DDOST |
| 1445 | IPI00136310 | | MRPL23 |
| 1446 | IPI00115580 | | EIF3M |
| 1447 | IPI00230427 | | Gm16379 |
| 1448 | IPI00128491 | | Aprt |
| 1449 | IPI00121288 | | NDUFB10 |
| 1450 | IPI00108895 | | psmc4 |
| 1451 | IPI00132722 | | LOC100045085 |
| 1452 | IPI00109044 | | 2900073G15Rik |
| 1453 | IPI00113655 | | Gm6476 |
| 1454 | IPI00656325 | | Nsf |
| 1455 | IPI00119219 | | HSD17B12 |
| 1456 | IPI00122568 | | arhgdib |
| 1457 | IPI00875584 | | Gm9646 |
| 1458 | IPI00226891 | | myef2 |
| 1459 | IPI00850843 | | LOC100047183 |
| 1460 | IPI00223714 | | Hist1h1e |
| 1461 | IPI00120100 | | P4HA2 |
| 1462 | IPI00469218 | | lamp1 |
| 1463 | IPI00115117 | | Stoml2 |
| 1464 | IPI00119545 | | PFDN2 |
| 1465 | IPI00115564 | | SLC25A4 |
| 1466 | IPI00622235 | | LOC675857 |
| 1467 | IPI00124771 | | SLC25A3 |
| 1468 | IPI00322312 | | ARHGDIA |
| 1469 | IPI00114368 | | SEC22B |
| 1470 | IPI00874482 | | Actg-ps1 |
| 1471 | IPI00123342 | | hyou1 |
| 1472 | IPI00323819 | | Gm6440 |
| 1473 | IPI00124096 | | Hiatl1 |
| 1474 | IPI00135512 | | Cnpy2 |
| 1475 | IPI00315135 | | Gm12906 |
| 1476 | IPI00761713 | | Hist1h2bp |
| 1477 | IPI00473728 | | |
| 1478 | IPI00225066 | | RPL36A |
| 1479 | IPI00130486 | | FKBP9 |
| 1480 | IPI00652813 | | fn1 |
| 1481 | IPI00134621 | | LOC100045999 |
| 1482 | IPI00121788 | | Gm7204 |
| 1483 | IPI00331597 | | Hist1h1d |
| 1484 | IPI00127415 | | LOC100046628 |
| 1485 | IPI00125971 | | Psmc6 |
| 1486 | IPI00222550 | | Gm4149 |
| 1487 | IPI00111218 | | ALDH2 |
| 1488 | IPI00551236 | | Stmn1-rs2 |
| 1489 | IPI00228616 | | Hist1h1a |
| 1490 | IPI00131954 | | D17Wsu104e |
| 1491 | IPI00135869 | | RAB11B |
| 1492 | IPI00416279 | | GLIPR2 |
| 1493 | IPI00130840 | | cope |
| 1494 | IPI00666885 | | DNAJC13 |
| 1495 | IPI00132276 | | vamp3 |
| 1496 | IPI00121079 | | cyb5r3 |
| 1497 | IPI00169925 | | Ndufv2 |
| 1498 | IPI00135475 | | DBN1 |
| 1499 | IPI00308984 | | EIF1AY |
| 1500 | IPI00407130 | | Gm6560 |
| 1501 | IPI00119138 | | Uqcrc2 |
| 1502 | IPI00132456 | | 2700060E02Rik |
| 1503 | IPI00130353 | | vars |
| 1504 | IPI00132334 | | 2610030H06Rik |
| 1505 | IPI00228633 | | gpi1 |
| 1506 | IPI00135186 | | CALU |
| 1507 | IPI00317902 | | PSMB5 |
| 1508 | IPI00121149 | | |
| 1509 | IPI00331121 | | Gm7379 |
| 1510 | IPI00111265 | | Capza2 |
| 1511 | IPI00226993 | | Txn1 |
| 1512 | IPI00331092 | | Gm8729 |
| 1513 | IPI00473521 | | Gm9058 |
| 1514 | IPI00323820 | | mcm2 |
| 1515 | IPI00129512 | | PSMB4 |
| 1516 | IPI00421223 | | Gm7809 |
| 1517 | IPI00856490 | | PSMD8 |
| 1518 | IPI00407917 | | Gm10117 |
| 1519 | IPI00321190 | | PSAP |
| 1520 | IPI00223217 | | Flt3l |
| 1521 | IPI00474883 | | CAPZB |
| 1522 | IPI00311236 | | Gm4734 |
| 1523 | IPI00120886 | | Gm6540 |
| 1524 | IPI00130304 | | BAG2 |
| 1525 | IPI00319830 | | Spnb2 |
| 1526 | IPI00117167 | | Gsn |
| 1527 | IPI00133249 | | SURF4 |
| 1528 | IPI00129517 | | Prdx5 |
| 1529 | IPI00128023 | | ndufs2 |
| 1530 | IPI00555069 | | Pgk1-rs7 |
| 1531 | IPI00114593 | | Actc1 |
| 1532 | IPI00124692 | | taldo1 |
| 1533 | IPI00271986 | | ATP5J2 |
| 1534 | IPI00317309 | | ANXA5 |
| 1535 | IPI00133215 | | Ndufb7 |
| 1536 | IPI00409223 | | Gm5265 |
| 1537 | IPI00323130 | | TCEB1 |
| 1538 | IPI00315302 | | Ndufa2 |
| 1539 | IPI00132756 | | ANXA8 |
| 1540 | IPI00620156 | | LOC100047329 |
| 1541 | IPI00270877 | | USP14 |
| 1542 | IPI00314467 | | PSMB3 |
| 1543 | IPI00319231 | | Gm5508 |
| 1544 | IPI00310131 | | Ap2a2 |
| 1545 | IPI00874935 | | LOC100046821 |
| 1546 | IPI00273803 | | Gm8885 |
| 1547 | IPI00420745 | | psmA2 |
| 1548 | IPI00225201 | | iars |
| 1549 | IPI00408892 | | Rab7 |
| 1550 | IPI00554894 | | ANXA6 |
| 1551 | IPI00283671 | | 2500003M10Rik |
| 1552 | IPI00224575 | | Gm7964 |
| 1553 | IPI00466820 | | Gm7246 |
| 1554 | IPI00420950 | | Gm12623 |
| 1555 | IPI00849670 | | Myof |
| 1556 | IPI00135640 | | PSMC5 |
| 1557 | IPI00114560 | | Rab1 |
| 1558 | IPI00119220 | | Gm5449 |
| 1559 | IPI00129685 | | Gm1974 |
| 1560 | IPI00118344 | | UGDH |
| 1561 | IPI00408207 | | MYO1D |
| 1562 | IPI00116154 | | Gm11273 |
| 1563 | IPI00133110 | | TXNDC12 |
| 1564 | IPI00225390 | | COX6B1 |
| 1565 | IPI00354819 | | Gm8894 |
| 1566 | IPI00131176 | | COX2 |
| 1567 | IPI00119478 | | tmod3 |
| 1568 | IPI00227838 | | GNG12 |
| 1569 | IPI00754649 | | LOC100048853 |
| 1570 | IPI00120719 | | COX5A |
| 1571 | IPI00223092 | | HADHA |
| 1572 | IPI00314748 | | wdr1 |
| 1573 | IPI00222188 | | COL1A2 |
| 1574 | IPI00131407 | | psma5 |
| 1575 | IPI00117829 | | cav1 |
| 1576 | IPI00462291 | | Gm13237 |
| 1577 | IPI00170093 | | Ndufs8 |
| 1578 | IPI00127408 | | rac1 |
| 1579 | IPI00130344 | | CLIC1 |
| 1580 | IPI00230715 | | Ndufa13 |
| 1581 | IPI00120503 | | COPB1 |
| 1582 | IPI00132728 | | cyc1 |
| 1583 | IPI00467172 | | LOC100048853 |
| 1584 | IPI00853924 | | LOC674211 |
| 1585 | IPI00467841 | | Gm7308 |
| 1586 | IPI00331332 | | NDUFA5 |
| 1587 | IPI00466570 | | tmed10 |
| 1588 | IPI00115977 | | ME2 |
| 1589 | IPI00132042 | | Gm6123 |
| 1590 | IPI00138691 | | ARPC4 |
| 1591 | IPI00399959 | | P4HA1 |
| 1592 | IPI00111885 | | Uqcrc1 |
| 1593 | IPI00137331 | | CAP1 |
| 1594 | IPI00133066 | | PSMD12 |
| 1595 | IPI00133240 | | uqcrfs1 |
| 1596 | IPI00133163 | | Gm13453 |
| 1597 | IPI00308882 | | NDUFS1 |
| 1598 | IPI00408119 | | Mtap4 |
| 1599 | IPI00118930 | | napA |
| 1600 | IPI00344004 | | NDUFA12 |
| 1601 | IPI00228617 | | gnai2 |
| 1602 | IPI00119111 | | Gm4815 |
| 1603 | IPI00554989 | | Gm9234 |
| 1604 | IPI00130589 | | Gm8566 |
| 1605 | IPI00329872 | | COL1A1 |
| 1606 | IPI00130240 | | ppiC |
| 1607 | IPI00462445 | | NEDD4 |
| 1608 | IPI00222515 | | Psmd11 |
| 1609 | IPI00125267 | | vapa |
| 1610 | IPI00648927 | | CLTA |
| 1611 | IPI00314439 | | psmd3 |
| 1612 | IPI00169870 | | GLT25D1 |
| 1613 | IPI00453777 | | Atp5d |
| 1614 | IPI00225961 | | Gm5207 |
| 1615 | IPI00114945 | | 41154 |
| 1616 | IPI00139780 | | LOC100044627 |
| 1617 | IPI00121427 | | S100A6 |
| 1618 | IPI00224210 | | UQCRQ |
| 1619 | IPI00314950 | | Gm9093 |
| 1620 | IPI00378120 | | glrx5 |
| 1621 | IPI00222553 | | Gm10126 |
| 1622 | IPI00139795 | | LOC100048062 |
| 1623 | IPI00454142 | | 41163 |
| 1624 | IPI00125929 | | Ndufa4 |
| 1625 | IPI00137736 | | Gm3511 |
| 1626 | IPI00322562 | | Gm6204 |
| 1627 | IPI00666161 | | LOC674215 |
| 1628 | IPI00228113 | | MTHFD1L |
| 1629 | IPI00462072 | | Gm5506 |
| 1630 | IPI00622160 | | LOC676151 |
| 1631 | IPI00331644 | | Psma3 |
| 1632 | IPI00474487 | | Gm10168 |
| 1633 | IPI00110753 | | Gm7172 |
| 1634 | IPI00132217 | | FIS1 |
| 1635 | IPI00470152 | | Gm5928 |
| 1636 | IPI00221540 | | Erlin2 |
| 1637 | IPI00132460 | | Gm7606 |
| 1638 | IPI00626233 | | Gm8358 |
| 1639 | IPI00460103 | | Gm7857 |
| 1640 | IPI00131406 | | PSMA7 |
| 1641 | IPI00122426 | | Gm9188 |
| 1642 | IPI00944009 | | Gm11675 |
| 1643 | IPI00459353 | | Gm3150 |
| 1644 | IPI00153660 | | dlaT |
| 1645 | IPI00129430 | | LOC100045887 |
| 1646 | IPI00126048 | | PSMD13 |
| 1647 | IPI00473429 | | Gm10051 |
| 1648 | IPI00127598 | | Atpif1 |
| 1649 | IPI00122549 | | VDAC1 |
| 1650 | IPI00473445 | | LOC100046290 |
| 1651 | IPI00117896 | | LOC674193 |
| 1652 | IPI00751369 | | LdhA |
| 1653 | IPI00453924 | | RPL37 |
| 1654 | IPI00133440 | | 1700071K01Rik |
| 1655 | IPI00120232 | | NDUFS7 |
| 1656 | IPI00378063 | | AP2B1 |
| 1657 | IPI00122547 | | vdac2 |
| 1658 | IPI00469194 | | tpp2 |
| 1659 | IPI00313222 | | Gm5428 |
| 1660 | IPI00331524 | | ERLIN1 |
| 1661 | IPI00230623 | | Gm6520 |
| 1662 | IPI00121623 | | Gm11582 |
| 1663 | IPI00955167 | | |
| 1664 | IPI00108338 | | MCM3 |
| 1665 | IPI00323357 | | LOC641192 |
| 1666 | IPI00221463 | | Hist3h2a |
| 1667 | IPI00515257 | | Gm7973 |
| 1668 | IPI00850259 | | Gm14176 |
| 1669 | IPI00222549 | | Gm8808 |
| 1670 | IPI00312128 | | TRIM28 |
| 1671 | IPI00339916 | | LOC633677 |
| 1672 | IPI00626994 | | LOC100044315 |
| 1673 | IPI00453819 | | LARS |
| 1674 | IPI00123557 | | RUVBL2 |
| 1675 | IPI00321718 | | PHB2 |
| 1676 | IPI00475378 | | Gm4900 |
| 1677 | IPI00117312 | | Got2 |
| 1678 | IPI00134809 | | DLST |
| 1679 | IPI00132799 | | c1qbp |
| 1680 | IPI00474888 | | Ptgis |
| 1681 | IPI00173160 | | Gm4180 |
| 1682 | IPI00109082 | | DAD1 |
| 1683 | IPI00120871 | | Trmt112-ps |
| 1684 | IPI00114401 | | emd |
| 1685 | IPI00875405 | | PSMD1 |
| 1686 | IPI00113141 | | CS |
| 1687 | IPI00320208 | | Eef1b2 |
| 1688 | IPI00126913 | | atad3a |
| 1689 | IPI00420363 | | Gm12183 |
| 1690 | IPI00318841 | | LOC100047986 |
| 1691 | IPI00849165 | | Gm5789 |
| 1692 | IPI00957027 | | |
| 1693 | IPI00466069 | | Gm13050 |
| 1694 | IPI00114667 | | psmd7 |
| 1695 | IPI00459725 | | idh3a |
| 1696 | IPI00850217 | | UBE2N |
| 1697 | IPI00330303 | | LOC100046995 |
| 1698 | IPI00132169 | | TIMM8B |
| 1699 | IPI00308706 | | Gm7625 |
| 1700 | IPI00315794 | | LOC100047577 |
| 1701 | IPI00134484 | | Timm13 |
| 1702 | IPI00308885 | | Gm12141 |
| 1703 | IPI00321978 | | RanBP1 |
| 1704 | IPI00111959 | | CTPS |
| 1705 | IPI00356904 | | LOC100045542 |
| 1706 | IPI00853914 | | Gm5778 |
| 1707 | IPI00307837 | | Gm7161 |
| 1708 | IPI00230113 | | CYB5 |
| 1709 | IPI00124973 | | Gm7379 |
| 1710 | IPI00136382 | | Sdc4 |

TABLE 2
Amplified Targets

| Number | GeneSymbol |
|---|---|
| 1 | GMPS |
| 2 | GFM1 |
| 3 | KPNA4 |
| 4 | NCEH1 |
| 5 | MRPL47 |
| 6 | PSMD2 |
| 7 | FXR1 |
| 8 | TFRC |
| 9 | UBXN7 |
| 10 | POP1 |
| 11 | MTDH |
| 12 | PABPC1 |
| 13 | YWHAZ |
| 14 | PLEC |
| 15 | FAM49B |
| 16 | PUF60 |
| 17 | TSTA3 |
| 18 | PSMB4 |
| 19 | TAGLN2 |
| 20 | HIST2H2BF |
| 21 | PSMD4 |
| 22 | PFDN2 |
| 23 | UAP1 |
| 24 | PRKACA |
| 25 | TPM4 |
| 26 | RAB8A |
| 27 | RAD23A |
| 28 | PPT1 |
| 29 | MYCBP |
| 30 | PABPC4 |
| 31 | LETM1 |
| 32 | TACC3 |
| 33 | MIB1 |
| 34 | SNRPD1 |
| 35 | PDCD5 |
| 36 | PSMD8 |
| 37 | PROSC |
| 38 | NDUFB9 |

### Components of the Riboproteome

The one or more components of the riboproteome described herein can include, but are not limited to ribosomal protein of the small ribosome subunit and ribosomal protein of the large ribosome subunit. A list of the human ribosomal protein genes is presented in Table 3.

TABLE 3
Ribosomal proteins of the small and large subunit

| Small Subunit | | Large Subunit | |
|---|---|---|---|
| SA | RPSA | L3 | RPL3 |
| S2 | RPS2 | L4 | RPL4 |
| S3 | RPS3 | L5 | RPL5 |
| S3A | RPS3A | L6 | RPL6 |
| S4 | RPS4X | L7 | RPL7 |
| | RPS4Y | L7A | RPL7A |
| S5 | RPS5 | L8 | RPL8 |
| S6 | RPS6 | L9 | RPL9 |
| S7 | RPS7 | L10 | RPL10 |
| S8 | RPS8 | L10A | RPL10A |
| S9 | RPS9 | L11 | RPL11 |
| S10 | RPS10 | L12 | RPL12 |
| S11 | RPS11 | L13 | RPL13 |
| S12 | RPS12 | L13A | RPL13A |
| S13 | RPS13 | L14 | RPL14 |
| S14 | RPS14 | L15 | RPL15 |
| S15 | RPS15 | L17 | RPL17 |
| S15A | RPS15A | L18 | RPL18 |
| S16 | RPS16 | L18A | RPL18A |
| S17 | RPS17 | L19 | RPL19 |
| S18 | RPS18 | L21 | RPL21 |
| S19 | RPS19 | L22 | RPL22 |
| S20 | RPS20 | L23 | RPL23 |
| S21 | RPS21 | L23A | RPL23A |
| S23 | RPS23 | L24 | RPL24 |
| S24 | RPS24 | L26 | RPL26 |
| S25 | RPS25 | L27 | RPL27 |
| S26 | RPS26 | L27A | RPL27A |
| S27 | RPS27 | L28 | RPL28 |
| S27A | RPS27A | L29 | RPL29 |
| S28 | RPS28 | L30 | RPL30 |
| S29 | RPS29 | L31 | RPL31 |
| S30 | RPS30 | L32 | RPL32 |
| | | L34 | RPL34 |
| | | L35 | RPL35 |
| | | L35A | RPL35A |
| | | L36 | RPL36 |
| | | L36A | RPL36A |
| | | L37 | RPL37 |
| | | L37A | RPL37A |
| | | L38 | RPL38 |
| | | L39 | RPL39 |
| | | L40 | RPL40 |
| | | L41 | RPL41 |
| | | LP0 | RPLP0 |
| | | LP1 | RPLP1 |
| | | LP2 | RPLP2 |
| | | LP3 | |

The one or more components of the riboproteome can also be a protein that is a known translation-associated protein including, for example, initiation and elongation factors, and proteins that are conserved across various species and cell lines, such as those listed in Table 4.

TABLE 4
Conserved Riboproteome Components

| Number | IPI.Prot | GeneSymbol | DAVID.Official |
|---|---|---|---|
| 1 | IPI00008223 | RAD23B | RAD23B |
| 2 | IPI00218918 | ANX1 | ANXA1 |
| 3 | IPI00017334 | PHB | Phb |
| 4 | IPI00008527 | RPLP1 | rplp1 |
| 5 | IPI00169383 | MIG10 | PGK1 |
| 6 | IPI00553185 | CCT3 | Cct3 |
| 7 | IPI00376005 | EIF5A | eif5a |
| 8 | IPI00015018 | IOPPP | PPA1 |
| 9 | IPI00154975 | DNAJC9 | dnajc9 |
| 10 | IPI00100160 | CAND1 | CAND1 |
| 11 | IPI00024911 | C12orf8 | ERP29 |
| 12 | IPI00939304 | IPO5 | IPO5 |
| 13 | IPI00219160 | RPL34 | Rpl34 |
| 14 | IPI00014197 | CDV3 | CDV3 |
| 15 | IPI00007402 | IPO7 | IPO7 |
| 16 | IPI00022305 | BZW2 | BZW2 |
| 17 | IPI00021405 | LMN1 | lmna |
| 18 | IPI00021187 | INO80H | Ruvbl1 |
| 19 | IPI00002520 | SHMT2 | SHMT2 |
| 20 | IPI00017596 | MAPRE1 | Mapre1 |
| 21 | IPI00298994 | KIAA1027 | TLN1 |
| 22 | IPI00303882 | M6PRBP1 | PLIN3 |
| 23 | IPI00909195 | C16orf34 | HN1L |
| 24 | IPI00031691 | OK/SW-cl.103 | rpl9 |
| 25 | IPI00298961 | CRM1 | xpo1 |
| 26 | IPI00217563 | FNRB | Itgb1 |
| 27 | IPI00220301 | AOP2 | Prdx6 |
| 28 | IPI00218019 | BSG | bsg |
| 29 | IPI00465028 | hCG_25936 | TPI1 |
| 30 | IPI00216694 | PLS3 | PLS3 |
| 31 | IPI00221089 | RPS13 | rps13 |
| 32 | IPI00299571 | PDIA6 | pdia6 |
| 33 | IPI00301263 | CAD | cad |
| 34 | IPI00215911 | APE | Apex1 |
| 35 | IPI00001757 | HSPC114 | RBM8A |
| 36 | IPI00013122 | CDC37 | cdc37 |
| 37 | IPI00099550 | DA41 | Ubqln1 |
| 38 | IPI00900293 | FLNB | FLNB |
| 39 | IPI00009342 | IQGAP1 | IQGAP1 |
| 40 | IPI00022648 | EIF5 | EIF5 |
| 41 | IPI00218606 | RPS23 | rps23 |
| 42 | IPI00294834 | ASPH | asph |
| 43 | IPI00002460 | ANX7 | Anxa7 |
| 44 | IPI00021700 | PCNA | pcnA |
| 45 | IPI00023860 | NAP1L1 | NAP1L1 |
| 46 | IPI00006579 | COX4 | COX4I1 |
| 47 | IPI00024067 | CLH17 | CLTC |
| 48 | IPI00031461 | GDI2 | GDI2 |
| 49 | IPI00550020 | PTMS | PTMS |
| 50 | IPI00376798 | RPL11 | rpl11 |
| 51 | IPI00008240 | MARS | mars |
| 52 | IPI00216691 | PFN1 | pfn1 |
| 53 | IPI00549725 | CDABP0006 | pgam1 |
| 54 | IPI00419258 | HMG1 | HMGB1 |
| 55 | IPI00219077 | LTA4 | LTA4H |
| 56 | IPI00020984 | CANX | Canx |
| 57 | IPI00220835 | SEC61B | SEC61B |
| 58 | IPI00007765 | GRP75 | HSPA9 |
| 59 | IPI00219156 | RPL30 | RpL30 |
| 60 | IPI00789551 | MATR3 | MATR3 |
| 61 | IPI00017448 | RPS21 | rps21 |
| 62 | IPI00185919 | KIAA0731 | LARP1 |
| 63 | IPI00003918 | RPL1 | rpl4 |
| 64 | IPI00022462 | TFRC | TFRC |
| 65 | IPI00220642 | YWHAG | YWHAG |
| 66 | IPI00470498 | CGI-55 | serbp1 |
| 67 | IPI00140420 | SND1 | SND1 |
| 68 | IPI00009943 | RP11-290D2.1-004 | TPT1 |
| 69 | IPI00221222 | PC4 | sub1 |
| 70 | IPI00883857 | HNRNPU | Hnrnpu |
| 71 | IPI00220362 | HSPE1 | HSPE1 |
| 72 | IPI00012442 | G3BP | G3BP1 |
| 73 | IPI00302927 | CCT4 | cct4 |
| 74 | IPI00010796 | ERBA2L | p4hb |
| 75 | IPI00025252 | ERP57 | PDIA3 |
| 76 | IPI00025512 | HSP27 | Hspb1 |
| 77 | IPI00299000 | EBP1 | PA2G4 |
| 78 | IPI00328753 | CG1 | Ktn1 |
| 79 | IPI00396485 | EEF1A | EEF1A1 |
| 80 | IPI00021812 | AHNAK | ahnak |
| 81 | IPI00025091 | RPS11 | rps11 |
| 82 | IPI00479191 | HNRNPH1 | Hnrnph1 |
| 83 | IPI00396321 | LRRC59 | LRRC59 |
| 84 | IPI00555744 | RPL14 | rpl14 |
| 85 | IPI00795671 | hCG_1744585 | Ran |
| 86 | IPI00010740 | PSF | sfpq |
| 87 | IPI00873899 | ABC50 | ABCF1 |
| 88 | IPI00783872 | CAPRIN1 | Caprin1 |
| 89 | IPI00008433 | RPS5 | Rps5 |
| 90 | IPI00216298 | TRDX | TXN |
| 91 | IPI00020599 | CALR | CALR |
| 92 | IPI00163187 | FAN1 | fscn1 |
| 93 | IPI00646304 | CYPB | ppiB |
| 94 | IPI00009904 | ERP70 | Pdia4 |
| 95 | IPI00479786 | FUBP2 | khsrp |
| 96 | IPI00290279 | ADK | adk |
| 97 | IPI00953576 | IGF2BP2 | Igf2bp2 |
| 98 | IPI00013894 | STIP1 | stip1 |
| 99 | IPI00642904 | hCG_2031827 | PABPC4 |
| 100 | IPI00333541 | FLN | FLNA |
| 101 | IPI00219678 | EIF2A | EIF2S1 |
| 102 | IPI00306332 | RPL24 | rpl24 |
| 103 | IPI00027107 | TUFM | Tufm |
| 104 | IPI00029012 | EIF3A | eif3a |
| 105 | IPI00783271 | LRP130 | lrpprc |
| 106 | IPI00014263 | EIF4H | eif4h |
| 107 | IPI00219153 | RPL22 | rpl22 |
| 108 | IPI00414676 | HSP90AB1 | HSP90AB1 |
| 109 | IPI00009328 | DDX48 | Eif4a3 |
| 110 | IPI00219155 | RPL27 | Rpl27 |
| 111 | IPI00514856 | KIAA0144 | Ubap2l |
| 112 | IPI00219446 | PBP | PEBP1 |
| 113 | IPI00550021 | OK/SW-cl.32 | RPL3 |
| 114 | IPI00008524 | PAB1 | PABPC1 |
| 115 | IPI00399265 | hCG_22755 | TPD52L2 |
| 116 | IPI00010153 | RPL23 | rpl23 |
| 117 | IPI00301936 | ELAVL1 | ELAVL1 |
| 118 | IPI00926625 | ZYX | ZYX |
| 119 | IPI00016610 | PCBP1 | Pcbp1 |
| 120 | IPI00915363 | RPS24 | RPS24 |
| 121 | IPI00183626 | hCG_20560 | Ptbp1 |
| 122 | IPI00011253 | OK/SW-cl.26 | rps3 |
| 123 | IPI00026781 | FAS | FASN |
| 124 | IPI00396378 | HNRNPA2B1 | HNRNPA2B1 |
| 125 | IPI00645078 | A1S9T | Uba1 |
| 126 | IPI00304596 | NONO | nonO |
| 127 | IPI00020956 | HDGF | hdgf |
| 128 | IPI00465361 | BBC1 | RPL13 |
| 129 | IPI00024157 | FKBP25 | FKBP3 |
| 130 | IPI00017617 | DDX5 | DDX5 |
| 131 | IPI00025874 | RPN1 | RPN1 |
| 132 | IPI00029731 | GIG33 | RpL35A |
| 133 | IPI00848226 | GNB2L1 | GNB2L1 |
| 134 | IPI00000874 | PAGA | PRDX1 |
| 135 | IPI00013917 | RPS12 | rps12 |
| 136 | IPI00219219 | LGALS1 | LGALS1 |
| 137 | IPI00291006 | MDH2 | mdh2 |
| 138 | IPI00186290 | EEF2 | Eef2 |
| 139 | IPI00012069 | DIA4 | nqo1 |
| 140 | IPI00843975 | EZR | EZR |
| 141 | IPI00027626 | CCT6 | cct6a |
| 142 | IPI00171903 | HNRNPM | hnrnpm |
| 143 | IPI00604620 | NCL | ncl |
| 144 | IPI00003865 | HSC70 | HSPA8 |
| 145 | IPI00026202 | RPL18A | RpL18A |
| 146 | IPI00465248 | ENO1 | eno1 |
| 147 | IPI00031812 | NSEP1 | ybx1 |
| 148 | IPI00029744 | SSBP | SSBP1 |
| 149 | IPI00440493 | ATP5A | Atp5a1 |
| 150 | IPI00141318 | CKAP4 | Ckap4 |
| 151 | IPI00001159 | GCN1L1 | gcn1l1 |
| 152 | IPI00419979 | PAK2 | Pak2 |
| 153 | IPI00013452 | EPRS | eprs |
| 154 | IPI00022744 | CAS | CSE1L |
| 155 | IPI00856098 | RRBP1 | RRBP1 |
| 156 | IPI00022774 | VCP | Vcp |
| 157 | IPI00298547 | PARK7 | PARK7 |
| 158 | IPI00937615 | EEF1G | tut1 |
| 159 | IPI00029601 | CTTN | cttn |
| 160 | IPI00303476 | ATP5B | ATP5B |
| 161 | IPI00011200 | PGDH3 | PHGDH |
| 162 | IPI00647915 | CDABP0035 | TAGLN2 |
| 163 | IPI00219365 | MSN | msn |
| 164 | IPI00796333 | ALDA | ALDOA |
| 165 | IPI00215780 | RPS19 | rps19 |
| 166 | IPI00019502 | MYH9 | MYH9 |
| 167 | IPI00643920 | TKT | tkt |
| 168 | IPI00018146 | YWHAQ | ywhaq |
| 169 | IPI00001639 | KPNB1 | KPNB1 |
| 170 | IPI00215790 | RPL38 | RPL38 |
| 171 | IPI00305064 | CD44 | CD44 |
| 172 | IPI00456758 | RPL27A | RPL27A |
| 173 | IPI00007423 | ANP32B | anp32b |
| 174 | IPI00219301 | MACS | MARCKS |
| 175 | IPI00219217 | LDHB | ldhb |
| 176 | IPI00010182 | DBI | DBI |
| 177 | IPI00216318 | YWHAB | YWHAB |
| 178 | IPI00952583 | MDH1 | Mdh1 |
| 179 | IPI00012750 | RPS25 | RPS25 |
| 180 | IPI00414860 | RPL37A | RPL37A |
| 181 | IPI00026271 | PRO2640 | rps14 |
| 182 | IPI00060181 | EFHD2 | Efhd2 |
| 183 | IPI00020436 | RAB11B | RAB11B |
| 184 | IPI00793199 | ANX4 | anxa4 |
| 185 | IPI00003949 | BLU | UBE2N |
| 186 | IPI00000684 | SPAG2 | UAP1 |
| 187 | IPI00216026 | VDAC2 | vdac2 |
| 188 | IPI00219344 | BDR1 | hpcal1 |
| 189 | IPI00479997 | LAP18 | STMN1 |
| 190 | IPI00165393 | ANP32E | ANP32E |
| 191 | IPI00024933 | RPL12 | RPL12P6 |
| 192 | IPI00000643 | BAG2 | BAG2 |
| 193 | IPI00382470 | HSP90A | HSP90AA2 |
| 194 | IPI00419585 | CYPA | PPIAL3 |
| 195 | IPI00784154 | HSP60 | HSPD1P6 |
| 196 | IPI00412607 | RPL35 | RPL35P2 |
| 197 | IPI00028635 | RPN2 | Rpn2 |
| 198 | IPI00216237 | RPL36 | RPL36P14 |
| 199 | IPI00007797 | FABP5 | FABP5L8 |
| 200 | IPI00419919 | RPL29 | RPL29P11 |
| 201 | IPI00784090 | C21orf112 | CCT8P1 |
| 202 | IPI00008530 | RPLP0 | RPLP0P6 |
| 203 | IPI00646689 | TXNDC17 | Txndc17 |
| 204 | IPI00008274 | CAP | CAP1 |
| 205 | IPI00291467 | ANT3 | SLC25A6 |
| 206 | IPI00221035 | BTF3 | BTF3L1 |
| 207 | IPI00008494 | ICAM1 | ICAM1 |
| 208 | IPI00021428 | ACTA | ACTA1 |
| 209 | IPI00013890 | HME1 | SFN |
| 210 | IPI00016342 | RAB7 | RAB7A |
| 211 | IPI00013004 | C21orf124 | pdxK |
| 212 | IPI00221093 | RPS17 | rps17 |
| 213 | IPI00178440 | EEF1B | Eef1b2 |
| 214 | IPI00005202 | DG6 | PGRMC2 |
| 215 | IPI00026105 | SCP2 | SCP2 |
| 216 | IPI00028031 | ACADVL | ACADVL |
| 217 | IPI00239077 | HINT | HINT1 |
| 218 | IPI00828150 | SUGT1 | Sugt1 |
| 219 | IPI00305383 | UQCRC2 | Uqcrc2 |
| 220 | IPI00873344 | TPD52 | TPD52 |
| 221 | IPI00289499 | ATIC | ATIC |
| 222 | IPI00018206 | GOT2 | Got2 |
| 223 | IPI00026546 | PAFAH1B2 | Pafah1b2 |
| 224 | IPI00470528 | EC45 | RPL15P18 |
| 225 | IPI00927658 | PP9932 | SNORA7B |
| 226 | IPI00827535 | PTMA | PTMAP4 |
| 227 | IPI00413108 | LAMBR | RPSAP19 |
| 228 | IPI00182289 | RPS29 | RPS29P11 |
| 229 | IPI00013296 | D6S218E | RPS18P12 |
| 230 | IPI00013485 | RPS2 | RPS2P17 |
| 231 | IPI00479058 | RIG | RPS15P5 |
| 232 | IPI00293276 | GLIF | |
| 233 | IPI00973811 | TBCA | |
| 234 | IPI00955848 | hCG_1739142 | |
| 235 | IPI00893918 | DAAP-21F2.2-001 | |
| 236 | IPI00984795 | CFL | |
| 237 | IPI00815770 | SNX3 | snx3 |
| 238 | IPI00303318 | BM-009 | FAM49B |
| 239 | IPI00880007 | MAP4 | |
| 240 | IPI00983068 | GRIM12 | |
| 241 | IPI00719622 | RPS28 | |
| 242 | IPI00009946 | TOMM34 | TOMM34 |
| 243 | IPI00024915 | ACR1 | Prdx5 |
| 244 | IPI00746165 | WDR1 | wdr1 |
| 245 | IPI00026215 | FEN1 | FEN1 |
| 246 | IPI00479905 | NDUFB10 | NDUFB10 |
| 247 | IPI00026337 | RANBP3 | RANBP3 |
| 248 | IPI00029557 | GREPEL1 | GRPEL1 |
| 249 | IPI00018465 | CCT7 | Cct7 |
| 250 | IPI00297779 | 99D8.1 | CCT2 |
| 251 | IPI00010896 | CLIC1 | CLIC1 |
| 252 | IPI00032826 | FAM10A1 | LOC729992 |
| 253 | IPI00013949 | SGT | Sgta |
| 254 | IPI00438229 | KAP1 | TRIM28 |
| 255 | IPI00018352 | UCHL1 | Uchl1 |
| 256 | IPI00215914 | ARF1 | Arf1 |
| 257 | IPI00790342 | RPL6 | RPL6P27 |
| 258 | IPI00221088 | RPS9 | RPS9P4 |
| 259 | IPI00947127 | LDHA | LdhA |
| 260 | IPI00017672 | NP | pnp |
| 261 | IPI00247583 | RPL21 | RPL21P14 |
| 262 | IPI00013415 | RPS7 | RPS7P10 |
| 263 | IPI00221091 | OK/SW-cl.82 | RPS15AP12 |
| 264 | IPI00010810 | ETFA | etfA |
| 265 | IPI00334190 | HSPC108 | Stoml2 |
| 266 | IPI01020905 | RPL18 | |
| 267 | IPI00985353 | EEF1D | |
| 268 | IPI00025491 | DDX2A | EIF4A1P4 |
| 269 | IPI00221092 | RPS16 | RPS16P10 |
| 270 | IPI00018931 | MEM3 | Vps35 |
| 271 | IPI00013895 | MLN70 | S100A11 |
| 272 | IPI00329801 | ANX5 | ANXA5 |
| 273 | IPI00744692 | TAL | taldo1 |
| 274 | IPI00550746 | NUDC | nudC |
| 275 | IPI00299149 | SMT3B | SUMO3 |
| 276 | IPI00549343 | SYB3 | vamp3 |
| 277 | IPI00027463 | CACY | S100A6 |
| 278 | IPI00290566 | CCT1 | TCP1 |
| 279 | IPI00026154 | G19P1 | prkcsh |
| 280 | IPI00303207 | ABCE1 | ABCE1 |
| 281 | IPI00011229 | CPSD | CTSD |
| 282 | IPI00218733 | SOD1 | SOD1 |
| 283 | IPI00027252 | BAP | PHB2 |
| 284 | IPI00026328 | TLP19 | TXNDC12 |
| 285 | IPI00219913 | TGT | USP14 |
| 286 | IPI00017704 | CLP | cotl1 |
| 287 | IPI00397571 | NSFL1C | nsfl1c |
| 288 | IPI00001960 | CLIC4 | CLIC4 |
| 289 | IPI00554737 | PPP2R1A | PPP2R1A |
| 290 | IPI00000816 | YWHAE | LOC440917 |
| 291 | IPI00029997 | PGLS | pgls |
| 292 | IPI00105598 | PSMD11 | Psmd11 |
| 293 | IPI00237884 | AKAP12 | AKAP12 |
| 294 | IPI00025796 | NDUFS3 | ndufs3 |
| 295 | IPI00220766 | GLO1 | GLO1 |
| 296 | IPI00029133 | ATP5F1 | ATP5F1 |
| 297 | IPI00024993 | ECHS1 | Echs1 |
| 298 | IPI00789155 | CALU | CALU |
| 299 | IPI00003815 | ARHGDIA | ARHGDIA |
| 300 | IPI00000877 | GRP170 | hyou1 |
| 301 | IPI00014230 | C1QBP | c1qbp |
| 302 | IPI00414127 | RANBP1 | RanBP1 |
| 303 | IPI00025849 | ANP32A | LOC723972 |
| 304 | IPI00216746 | HNRNPK | LOC644063 |
| 305 | IPI00644127 | IARS | iars |
| 306 | IPI00216008 | G6PD | G6PD |
| 307 | IPI00307162 | VCL | vcl |
| 308 | IPI00220827 | PTMB10 | TMSB10 |
| 309 | IPI00018398 | PSMC3 | psmc3 |
| 310 | IPI00549248 | NPM | LOC399804 |
| 311 | IPI00103994 | KIAA1352 | LARS |
| 312 | IPI00216319 | YWHA1 | YWHAH |
| 313 | IPI00021805 | GST12 | mgst1 |
| 314 | IPI00215687 | GLS | GLS |
| 315 | IPI00025366 | CS | CS |
| 316 | IPI00640817 | AK1 | ak1 |
| 317 | IPI00218693 | APRT | Aprt |
| 318 | IPI00412579 | NEDD6 | RPL10AP9 |
| 319 | IPI00003362 | GRP78 | LOC400750 |
| 320 | IPI00216308 | VDAC | VDAC1P1 |
| 321 | IPI00012197 | CDA03 | DCTPP1 |
| 322 | IPI00219018 | CDABP0047 | LOC100133042 |
| 323 | IPI00217030 | CCG2 | RPS4XP13 |
| 324 | IPI00026302 | RPL31 | RPL31P17 |
| 325 | IPI00021266 | RPL23A | RPL23AP65 |
| 326 | IPI00021840 | OK/SW-cl.2 | RPS6P25 |
| 327 | IPI00299573 | RPL7A | RPL7AP30 |
| 328 | IPI00655650 | RPS26 | RPS26P8 |
| 329 | IPI00012772 | RPL8 | RPL8P2 |
| 330 | IPI00419880 | FTE1 | LOC100130107 |
| 331 | IPI00027270 | RPL26 | RPL26P33 |
| 332 | IPI00025329 | RPL19 | RPL19P12 |
| 333 | IPI00479186 | OIP3 | LOC652797 |
| 334 | IPI00304612 | RPL13A | RPL13AP7 |
| 335 | IPI00000494 | MSTP030 | RPL5P34 |
| 336 | IPI00008438 | RPS10 | RPS10P7 |
| 337 | IPI00216587 | OK/SW-cl.83 | RPS8P10 |
| 338 | IPI00030179 | RPL7 | RPL7P20 |
| 339 | IPI00179330 | RPS27A | RPS27AP11 |
| 340 | IPI00604590 | hCG_2001850 | NME2 |
| 341 | IPI00008529 | D11S2243E | RPLP2P3 |
| 342 | IPI00219953 | CMK | CMPK1 |
| 343 | IPI00395887 | PSEC0085 | tmx1 |
| 344 | IPI00219757 | FAEES3 | GSTP1 |
| 345 | IPI00418169 | ANX2 | ANXA2P1 |
| 346 | IPI00398009 | IMP4B | IPO4 |
| 347 | IPI00297579 | CBX3 | LOC644101 |
| 348 | IPI00017592 | LETM1 | LETM1 |
| 349 | IPI00291928 | RAB14 | Rab14 |
| 350 | IPI00025086 | COX5A | COX5A |
| 351 | IPI00075248 | CALM | CALM3 |
| 352 | IPI00219078 | ATP2A2 | ATP2A2 |
| 353 | IPI00295386 | CBR | Cbr1 |
| 354 | IPI00982652 | FAU | |
| 355 | IPI00010105 | EIF3A | |
| 356 | IPI00977661 | RPL17 | |
| 357 | IPI00007611 | ATP5O | |
| 358 | IPI01009955 | BCAP31 | |
| 359 | IPI01019113 | OK/SW-cl.56 | |
| 360 | IPI00797126 | HSD48 | |
| 361 | IPI00006865 | SEC22B | |
| 362 | IPI00171438 | TLP46 | |
| 363 | IPI01022506 | EIF4B | |

Screening Assays to Identify One or More Compounds that Modulate the Association Between the Target and Component of the Riboproteome

As discussed above, we have discovered that riboproteomic genes are frequently amplified in cancer. Based on these discoveries, the association between the target and the component of the riboproteome (e.g., including but not limited to those targets that bind to the components of the riboproteome directly or targets that interact with the component of the riboproteome by an interaction that is mediated by another macromolecule, (e.g., another protein or enzyme)) can be monitored as a way to identify candidate compounds that modulate, alter, increase or decrease (e.g., by at least 10%, 20%, 30%, 40%, 50%, 60%, 70%, 80%, 90%, 95%, or more) the association between the one or more targets and one or more components of the riboproteome. Compounds that modulate, alter, decrease, or increase the association between the one or more targets and the one or more components of the riboproteome can be used for the treatment or prevention of a cancer resulting from the change in the level of association between the target and the component of the riboproteome, e.g., prostate adenocarcinoma, ovarian serous cystadenocarcinoma, lung squamous cell carcinoma and any other conditions described herein.

In particular examples, candidate compounds having one or more of the following properties are considered modulators of the association between the target and the component of the riboproteome: decreased association between the target and the component of the riboproteome (e.g., from 3-fold to 4-fold decreased association), decreased expression of the target or the component of the riboproteome, decrease activity of the target or the component of the riboproteome, increased association between the target and the component of the riboproteome, increased expression of the target or the component of the riboproteome, or increased activity of the target or the component of the riboproteome, as compared to a control or a normal reference. Candidate compounds can be tested for their effect on modulation of the association between the target and the component of the riboproteome using assays known in the art.

Candidate compounds can also be tested for their modulation in the association between the target and the component of the riboproteome using any particular cell based assays described herein. Standard methods may be used to measure analyte levels or cellular parameters in any bodily fluid, including, but not limited to, urine, blood, serum, plasma, saliva, or cerebrospinal fluid. Such methods include immunoassay, ELISA, Western blotting using antibodies directed to one or more targets or one or more components of the riboproteome and quantitative enzyme immunoassay techniques. ELISA assays are the preferred method for measuring polypeptide levels. Accordingly, the measurement of antibodies specific to one or more targets or one or more components of the riboproteome in a subject may also be used to determine if a compound has effects on modulating the association between the target and the component of the riboproteome.

In one embodiment, a compound that affects the association between the target and the component of the riboproteome may show a decrease in the expression of a nucleic acid encoding the target or the component of the riboproteome. Methods for detecting such alterations are standard in the art. In one example Northern blotting or real-time PCR is used to detect mRNA levels.

In another embodiment, hybridization techniques may be used to monitor expression levels of a gene encoding the target or the component of the riboproteome upon treatment with a candidate compound.

In a further embodiment, a reporter gene such as a gene encoding GFP or luciferase can be fused to the target or the component of the riboproteome to monitor the expression levels of the target or the component of the riboproteome upon treatment with a candidate compound.

In general, candidate compounds are identified from large libraries of both natural product or synthetic (or semi-synthetic) extracts, chemical libraries, or from polypeptide or nucleic acid libraries, according to methods known in the art. Those skilled in the field of drug discovery and development will understand that the precise source of test extracts or compounds is not critical to the screening procedure(s) of the invention.

### Diagnostic Methods

The level of association between one or more targets and one or more components of the riboproteome can be used for the diagnosis of a particular type of cancer (e.g., the cancers resulting from a change in the level of association between the target and the component of the riboproteome, a cancer as described herein), or a risk of developing a cancer. The level of association between one or more targets and one or more components of the riboproteome can also be used to monitor the therapeutic efficacy of one or more compounds, including compounds identified or described herein, used to treat a cancer described herein.

The overall riboproteome profile obtained from a subject can also be used for diagnostic or prognostic purposes. Furthermore, combinations of specific proteins and examination of the global landscape of the one or more targets associated with the one or more components of the riboproteome can be predictive of the outcome or response to treatment with the compounds identified or described herein. For example, amplification, appearance, or disappearance in a gene, protein, combinations of genes, or combinations of proteins found in the riboproteome profile when compared to a normal reference may represent indicators that can help to classify and/or stratify specific patient subgroups that in turn can provide a diagnosis and/or prognosis for treatment of a cancer resulting from a change in the level of association between the target and the component of the riboproteome.

Standard methods may be used to measure analyte levels or cellular parameters in any bodily fluid, including, but not limited to, urine, blood, serum, plasma, saliva, or cerebrospinal fluid. Such methods include use of mass spectrometry, UV absorption spectroscopy, fluorescence spectroscopy, and quantitative enzyme kinetics techniques.

Diagnostic methods can include measurement of absolute levels of the one or more targets or the one or more components of the riboproteome as an indirect read out of the change in the level of association between the target and the component of the riboproteome. In any of the diagnostic methods, the level of the target or component of the riboproteome, can be measured at least two different times from the same subject and an alteration in the levels (e.g., by 10%, 20%, 30%, 40%, 50%, 60%, 70%, 80%, 90%, or more) over time is used as an indicator of a change in the level of association between the target and the component of the riboproteome and therefore an indicator of a particular type of cancer, or propensity to develop the same. It will be understood by the skilled artisan that for diagnostic methods that include comparing of the level of association between the target and the component of the riboproteome, to a reference level, particularly a prior sample taken from the same subject, a change over time with respect to the baseline level can be used as a diagnostic indicator of a change in the level of association and thus an indicator of a particular type of cancer, or a predisposition to develop the same. The diagnostic methods described herein can be used individually or in combination with any other diagnostic method described herein for a more accurate diagnosis of the presence of, severity of, or predisposition to a particular type of cancer resulting from the change in the level of association between the target and the component of the riboproteome.

### Compounds

For any of the methods described herein, the compound identified can be a chemotherapeutic agent (e.g., arsenic trioxide, cisplatin, carboplatin, chlorambucil, melphalan, nedaplatin, oxaliplatin, triplatin tetranitrate, satraplatin, imatinib, nilotinib, dasatinib, and radicicol, e.g., cisplatin), an immunomodulatory agent (e.g., methotrexate, leflunomide, cyclophosphamide, cyclosporine A, minocycline, azathioprine, an antibiotic (e.g., tacrolimus), methylprednisolone, a corticosteroid, a steroid, mycophenolate mofetil, rapamycin, mizoribine, deoxyspergualin, brequinar, a T cell receptor modulator, and a cytokine receptor modulator, e.g., methotrexate), an antiangiogenic agent (e.g., alitretinoin, beloranib, bevacizumab, cetuximab, endostatin (e.g., recombinant forms thereof), erlotinib, etrathiomolybdate, everolimus, imiquimod, interferon alfa (e.g., recombinant forms thereof), itraconazole, lenalidomide, pazopanib, sorafenib, sunitinib, suramin, temsirolimus, thalidomide, tivozanib, vandetanib, and vatalanib), a mitotic inhibitor (e.g., paclitaxel, vinorelbine, docetaxel, abazitaxel, ixabepilone, larotaxel, ortataxel, tesetaxel, vinblastine, vincristine, vinflunine, and vindesine, e.g., paclitaxel), a nucleoside analog (e.g., gemcitabine, azacitidine, capecitabine, carmofur, cladribine, clofarabine, cytarabine, decitabine, floxuridine, fludarabine, fluorouracil, mercaptopurine, pentostatin, tegafur, and thioguanine, e.g., gemcitabine), a DNA intercalating agent (e.g., doxorubicin, actinomycin, bleomycin, mitomycin, and plicamycin, e.g., doxorubicin), a topoisomerase analog (e.g., irinotecan, aclarubicin, amrubicin, belotecan, camptothecin, daunorubicin, epirubicin, etoposide, idarubicin, mitoxantrone, pirarubicin, pixantrone, rubitecan, teniposide, topotecan, valrubicin, and zorubicin, e.g., irinotecan), an antibody (e.g., monoclonal antibodies, such as alemtuzumab, bevacizumab, brentuximab vedotin, catumaxomab, cetuximab, denosumab, edrecolomab, ertumaxomab, gemtuzumab ozogamicin, ibritumomab, ibritumomab tiuxetan, ipilimumab, ofatumumab, panitumumab, rituximab, tositumomab, trastuzumab, as well as radiolabeled forms thereof, or any described herein), a cytokine (e.g., recombinant interferon alpha, interferon beta, interferon gamma, interleukin 2, interleukin 11, granulocyte colony-stimulating factor (G-CSF) and pegylated forms thereof, granulocyte macrophage colony-stimulating factor (GM-CSF), and methionyl human stem cell factor (SCF), as well as any described herein), a folate antimetabolite (e.g., pemetrexed, aminopterin, methotrexate, pralatrexate, and raltitrexed, e.g., pemetrexed), or other targeting agents (e.g., agents that target particular enzymes or proteins involved in cancer or agents that target particular organs or types of cancers), and combinations thereof.

For any of the methods described herein, the compound identified can also be an inhibitor of metabolic proteins. For example, the compound can be a hexokinase inhibitor (e.g., a hexokinase 2 (HK2) inhibitor (e.g., 2-deoxyglucose, halogenated derivatives of 2-deoxyglucose (e.g., 2-fluorodeoxyglucose), 5-thioglucose, 3-bromopyruvate (3-BrPA), 3-bromo-2-oxopropionate-1-propylester (3-BrOP), lonidamine, imatinib, meclofenoxate, O,O-diethyl O-3,5,6-trichloro-2-pyridyl phosphate, 7,8-dihydro-7,8-dihydroxybenzo(a)pyrene 9,10-oxide, antisense RNA, N-terminal oligopeptide of hexokinase II (e.g., MIASHLLAYFFTELN-amide (hexokinase II VDAC binding domain peptide; HXK2VBD) and RQIKIWFQNRRMKWKKMIASHLLAYFFTELN-amide), antifungal derivatives (e.g., clotrimazole and bifonazole), and D-mannoheptulose); a lactate dehydrogenase inhibitor (e.g., a lactate dehydrogenase A (LDH-A) inhibitor or a lactate dehydrogenase 5 (LDH-5) inhibitor (e.g., oxamate, gossypol, 3-hydroxyisoxazole-4-carboxylic acid (H ICA), 4-hydroxy-1,2,5-thiadiazole-3-carboxylic acid (HTCA), 3-dihydroxy-6-methyl-7-(phenylmethyl)-4-propylnaphthalene-1-carboxylic acid (FX11), 7,8-dihydro-7,8-dihydroxybenzo(a)pyrene 9,10-oxide, cyclosporine, lindane, antisense RNA (e.g., shRNA, nt 204-232 (L1, gattaca gttgttgggg ttggtgctgt tg), nt 737-765 (L2, tgtg gagtggtgtg aatgttgccg gcgtc), and nt 1161-1188 (L3, tcactgtcca ggctgcagca gggcttct) of NCBI Reference Sequence: NM_{—}010699)), 1-hydroxy-5-phenyl-1H-indole-2-carboxylic acid, 1-hydroxy-6-phenyl-1H-indole-2-carboxylic acid, and 1-hydroxy-6-phenyl-4-trifluoromethyl-1H-indole-2-carboxylic acid); a phosphofructokinase 2 or phosphofructo-2-kinase/fructose-2,6-bisphosphatase (PFK2/PFKFB3) inhibitor (e.g., 3-(3-pyridinyl)-1-(4-pyridinyl)-2-propen-1-one); a pyruvate kinase M2 (PKM2) inhibitor (e.g., 4R,7S,10R,13S,16R)-7-(4-aminobutyl)-N-[(3R)-1-amino-3-hydroxy-1-oxobutan-2-yI]-16-[[(2R)-2-amino-3-phenylpropanoyl]amino]-13-[(4-hydroxyphenyl)methyl]-10-(1H-indol-3-ylmethyl)-6,9,12,15-tetraoxo-1,2-dithia-5,8,11,14-tetrazacycloheptadecane-4-carboxamide (TLN-232/CAP-232), shikonin, and alkannin); a transketolase inhibitor (e.g., a transketolase-like enzyme 1 (TKTL1) inhibitor (e.g., oxythiamine and furazolidone)); a pyruvate dehydrogenase (PDH) inhibitor (e.g., oxythiamine); a pyruvate dehydrogenase kinase (PDK) inhibitor (e.g., dichloroacetate); a glucose-6-phosphate dehydrogenase (G6PG) inhibitor (e.g., 6-aminonicotinamide, imatinib, 2,2′-azobis(2-amidinopropane), 2,5-dihydroxybenzoic acid, aluminum phosphide, arjunolic acid, benzo(a)pyrene, benzoyl peroxide, calphostin C, cycloartenol, dactinomycin, dexamethasone, diethylnitrosamine, endosulfan, fenvalerate, ferric nitrilotriacetate, ferrous sulfate, glyoxylic acid, furantoin, phenobarbitol, quercetin, isotretinoin, and streptozocin); a GLUT inhibitor (e.g., fluorodeoxyglucose, including radiolabelled forms ([18F]-fluorodeoxyglucose), 2-deoxyglucose, phloretin, and silybin/silibinin); a proton transport inhibitor, such as a carbonic anhydrase-9 (CA9) inhibitor, a membrane-bound V-ATPase inhibitor, and a sodium-proton exchanger 1 (NH E1) inhibitor (e.g., paclitaxel, acetazolamide, cariporide, indisulam, girentuximab, esomeprazole, amiloride and derivatives thereof (5-(N-ethyl-Nisopropyl)amiloride (EIPA)); a monocarboxylate transporter (MCT) inhibitor, such as MCT1, MCT2, MCT3, or MCT4 inhibitors (e.g., α-cyano-4-hydroxycinnamate (CHC), AZD3965, or AR-C117977); a hypoxia-inducible factor 1 alpha (HIF-1 alpha) inhibitor (e.g., BAY87-2243, acriflavine, PX-478, tirapazamine, and an antisense oligonucleotide targeting HIF-1α (EZN-2968, 5′-TGGcaagcatccTGTa-3′, where upper case indicates LNA residues and lower case indicates DNA residues)); a c-Myc inhibitor (e.g., (5E)-5-[(4-ethylphenyl)methylidene]-2-sulfanylidene-1,3-thiazolidin-4-one (10058-F4) and quarfloxin/CX-3453); an AMP-activated protein kinase (AMPK) inhibitor (e.g., metformin); a glutamine inhibitor (e.g., phenylacetate); an asparagine inhibitor (e.g., asparaginase and pegasparaginase); an arginine inhibitor (e.g., arginine deaminase); a fatty acid synthase (FASN) inhibitor (e.g., orlistat, GSK837149A, and C75); and an ATP-citrate lyase (ACLY) inhibitor (e.g., 2-[(3S,5R)-5-[6-(2,4-dichlorophenyl)hexyl]-3-hydroxy-2-oxooxolan-3-yl]acetic acid (SB-204990), quercetin, and rutin).

### Conditions

In any of the embodiments described herein, the cancer resulting from a change in the level of association between the target and the component of the riboproteome includes non-solid cancers and solid cancers. Exemplary cancers include, but are not limited to leukemia (e.g., chronic myeloid leukemia, acute myeloid leukemia, acute lymphoblastic leukemia, and chronic lymphocytic leukemia), adenoid cystic carcinoma, bladder cancer (e.g., adenocarcinoma, sarcoma, small cell carcinoma, squamous cell carcinoma, and transitional cell carcinoma), bladder urothelial carcinoma, brain cancer (e.g., ependymoma, glioma, medulloblastoma, meningioma, teratoid rhabdoid tumor, and teratoma, brain lower grade glioma), breast cancer (e.g., breast ductal carcinoma, breast invasive carcinoma), cervical squamous cell carcinoma and endocervical adenocarcinoma, colon and rectum adenocarcinoma, glioblastoma, glioblastoma multiforme, head and neck squamous cell carcinoma, kidney chromophobe, kidney renal clear cell carcinoma, kidney renal papillary cell carcinoma, liver cancer (e.g., hepatocellular carcinoma, cholangiocarcinoma, and hemangioendothelioma), lung cancer (e.g., non-small cell lung cancer, small-cell lung cancer, carcinoid, sarcoma, squamous cell cancer, adenocarcinoma (e.g., papillary adenocarcinoma), lymphoid neoplasm diffuse large b-cell lymphoma, ovarian cancer (e.g., ovarian epithelial carcinoma and teratoma, ovarian serous cystadenocarcinoma), pancreatic adenocarcinoma, prostate adenocarcinoma (e.g., adenocarcinoma and prostatic intraepithelial neoplasia), renal cancer, sarcoma, skin cancer (e.g., basal cell carcinoma, squamous cell carcinoma, and malignant melanoma, skin cutaneous melanoma), stomach adenocarcinoma, testis cancer, thyroid carcinoma, and uterine corpus endometrial carcinoma

The compounds identified and described herein can also be used to treat cancers having one or more particular mutations that confer resistance to first-line antineoplastic agents. Exemplary cancers having mutations include non-small cell lung cancer having a T790M or a L747S mutation in EGFR kinase, a somatic activating mutation in the tyrosine-kinase pocket of EGFR (e.g., a deletion in exon 19 or a substitution in exon 21, e.g., L858R), or a mutation present in tyrosine kinase inhibitor-resistant cell line H1975; and brain cancer, breast cancer, colorectal cancer, lung cancer, and stomach cancer having a E542K, E545K, H1047R, P539R, or H1047L mutation in the PIK3CA gene (encoding a p110a of class IA of PI3K) (e.g., lung cancer having a H1047R mutation in PIK3CA).

Other features and advantages of the invention will be apparent from the following description and the claims.

### EXAMPLES

### Example 1

### Experimental Procedures

### SILAC Labeling and Mass Spectrometry

Metabolic labeling of prostate cell lines (PC3, PPC1, Du145, RWPE1 and PWR1E) and MEFs was carried out using either normal arginine and lysine or heavier isotopic variants of the two amino acids (L-Lysine 2HCL (U-13C6), L-Arginine HCL (U-13C6, U-N15N4)) (Ong et al., Mol. Cell Proteomics 1:376-386, 2002) using Invitrogen's SILAC-FLEX Media kits. SILAC labeled protein mixtures were run by SDS-PAGE and gel lanes were cut into 8 sections for overnight digestion at pH 8.0 with modified sequencing grade trypsin (Promega Corp.). Peptide mixtures were eluted and each gel section was analyzed separately by microcapillary liquid chromatography-tandem mass spectrometry (LC-MS/MS) using the EASY-nLC nanoflow HPLC (Thermo Fisher Scientific) with a 75 μm inner diameter×15 cm length Picofrit capillary column (New Objective, Inc.) self-packed with 5 μm Magic C₁₈ resin (Michrom Bioresources) coupled to a hybrid LTQ Orbitrap XL-ETD mass spectrometer (Thermo Fisher Scientific). The LTQ Orbitrap XL was operated in data-dependent acquisition (DDA) Top 5 mode (1 profile FT-MS spectrum followed by 6 centroided IT-MS/MS spectra). The resolution was 30,000 in FT-MS mode and MS/MS spectra were read out at low resolution in the LTQ XL ion trap. The gradient consisted of 3-38% acetonitrile in 0.1% formic acid (FA) at a flow rate of 300 nL/min for 75 min, 38-95% acetonitrile in 0.1% FA for 2 min and held at 95% acetonitrile in 0.1% FA at for 7 min followed by column re-equilibration for 10 min at 3% acetonitrile in 0.1% FA. MS/MS fragmentation spectra were searched for protein identification using the Andromeda search engine (www.andromeda-search.org) (Cox et al., Proteome Res. 10:1794-1805, 2011) against the reversed and concatenated IPI_HUMAN protein database (v3.87) (http://www.ebi.ac.uk/IPI/IPIhuman.html). Carbamidomethylation of cysteine was set as fixed modification and variable modifications were oxidation of methionine and protein N-acetylation. Raw files for SILAC ratio analysis from each experiment were combined and processed using MaxQuant v1.2.2.5 software (http://www.maxquant.org/) (Cox and Mann, Nat. Biotechnol. 26:1367-1372, 2008). Initial peptide mass tolerance was set to 12 ppm and fragment ion mass tolerance was set to 0.8 Da. Two missed cleavages were allowed and the minimal length required for a peptide was six amino acids. One unique peptide was required for high-confidence protein identifications and a minimum ratio count of two peptides (one unique and one razor) were required for SILAC ratio determination. The peptide and protein false discovery rates (FDR) were set to 0.01. Normalized SILAC ratios (H/L) were used for subsequent analysis.

### Polysome Isolation and Analysis

Polysome profiles were prepared from MEF and different prostate cancer cell lines as follows. MEF were seeded at 2×10⁶ cells/15 cm and PPC1, PC3, Du145, RWPE1 and PWR1E cells seeded at 10×10⁶ cells/15 cm dish and cultured overnight to ensure sub confluent cultures for polysome analysis. PPC1 cells were treated the following day with either DMSO, rapamycin (20 nM) or PP242 (500 nM) for three hours. For polysome preparation, cells were then incubated with cycloheximide at a final concentration of 100 μg/mL for a period of 15 mins. Plates were then washed with ice-cold PBS containing 100 μg/mL cycloheximide (PBS/CHX), scraped, and collected in ice-cold PBS/CHX. Cells were pelleted by centrifugation and subsequently lysed in polysome lysis buffer (20 mM Tris-HCl, pH 7.4; 5 mM MgCl₂; 150 mM NaCl; 1% Triton X-100; 1% Deoxycholate; 2.5 mM DTT; 200 U/mL RNasin; 100 μg/mL cycloheximide; 1× complete, EDTA-free protease inhibitor cocktail (Roche); 1× protease inhibitor set (without EDTA) (G-Biosciences); α₁-antitrypsin (EMD Biosciences) and incubated on ice for 10 min with occasional mixing. Extensive optimization of cell lysis was carried out to identify suitable lysis buffer conditions that completely blocked protein degradation from endogenous proteases, it was necessary to include the extensive array of protease inhibitors provided in the G-Bioscience protease inhibitor set. Lysates were centrifuged at 7,000 rpm for 5 min at 4° C., and the supernatant carefully removed. Protein concentrations for lysates were measured by Bradford assay, and equal amounts of protein loaded on a 15%-50% sucrose gradient containing 100 μg/mL cycloheximide, 0.2 mg/mL heparin and 1 mM DTT. Gradients were centrifuged at 36,000 rpm for 3 hrs at 4° C. in a Beckman SW40 rotor, and subsequently fractionated using an ISCO-Foxy Jr. fraction collector. Polysome profiles were reordered using a UA-6 absorbance detector connected to the fraction collector and measuring absorbance at 254 nm.

Puromycin-induced polysome dissociation was carried out by the addition of 1 mM puromycin directly to the lysis buffer lacking cycloheximide as previously described (Blobel and Sabatini, Proc Natl Acad Sci USA 68:390-394, 1971; Fuchs et al., J Mol Biol. 410:118-130, 2011). Briefly, following lysis, the samples were incubated at 37° C. for 15 min to dissociate ribosome-mRNA complexes. Lysates were centrifuged at 13,000 rpm for 5 min at 4° C., and the supernatant carefully removed and loaded on a 15%-50% sucrose gradient. Gradients were centrifuged and fractionated as described above.

### Bioinformatics Analysis of the Riboproteome

To cluster riboproteome experiments based on the riboproteins identified in each experiment, a Boolean matrix of riboproteome genes by riboproteome experiments was created, in which each entry in the matrix was a 1 if the row's gene was identified in the column's experiment, and a 0 otherwise. Clustering of the experiments was then performed using a binary distance measure to compute the distance matrix, and average linkage for hierarchical clustering, implemented with the R functions dist and hclust.

Venn diagrams indicating membership of riboproteome genes to SILAC experiments were created using the Vennerable package in R.

To identify functional gene sets enriched in the riboproteome genes, the riboproteome genes were uploaded to Ingenuity Pathway Analysis (www.ingenuity.com) and identified the top biological functions gene sets and canonical pathways gene sets enriched in the riboproteome gene set. To identify KEGG pathways specifically enriched in the subset of riboproteome genes identified in 5 of 5 experiments as compared with the set identified in only 1 of 5 experiments, the 5 of 5 experiments gene list was uploaded to DAVID and used the 1 of 5 experiments gene list as background. Gene ontology analysis was carried out using the online DAVID bioinformatics resource tool.

### TCGA-Based Analyses of Riboproteome Genomic Alterations Across Human Cancers

To analyze global patterns of copy number alterations in the riboproteome vs. the background protein-coding genome, the cgdsr package in R was used to download Gistic copy-number alteration calls from the 16 cancer types with available data for a large portion of the riboproteome (between 1661 and 1720 of the riboproteome genes analyzed for each cancer type, median=1675) and the background protein coding genome (19195 genes). For each cancer type, the number of homozygous deletions (GISTIC score=−2), hemizygous deletions (GISTIC score=−1), diploid (GISTIC score=0), low-level copy number gain (GISTIC score=1), and high-level amplification (GISTIC score=2) were recorded among the riboproteome genes and among the background genome. The proportion of each of the GISTIC scores observed among the riboproteome genes to the proportion observed among the background protein-coding genome using the function prop.test in R were compared. To visualize and summarize the distribution of the proportions of alterations across the cancer types, forest plots using the rmeta package were created.

After characterizing the global properties of riboproteome genomic alterations in human cancers, gene-level analyses was performed. At the time of analysis, the cBio Cancer Genomics Portal contained 5 published data sets and 15 provisional datasets from The Cancer Genome Atlas (TCGA) profiling efforts (Cerami et al., Cancer Discov 2:401-404, 2012). While the 5 published datasets contain mutation data, the provisional TCGA datasets do not.

These analyses were limited to the 532 riboproteome genes with valid data across 15 TCGA cancer types. For each gene, the proportion of cases of each cancer type that the gene showed homozygous deletion, hemizygous deletion, diploid, low-level amplification, and high-level amplification, based on GISTIC calls downloaded via cgdsr were computed. For each riboproteome gene, the maximum proportion of each type of alteration across the TCGA cancer types was computed. Riboproteome genes and locations of riboproteome genes undergoing frequent amplifications in cancer were visualized with Circos-like plots, implemented using ggplot in R.

### Western Blot Analysis

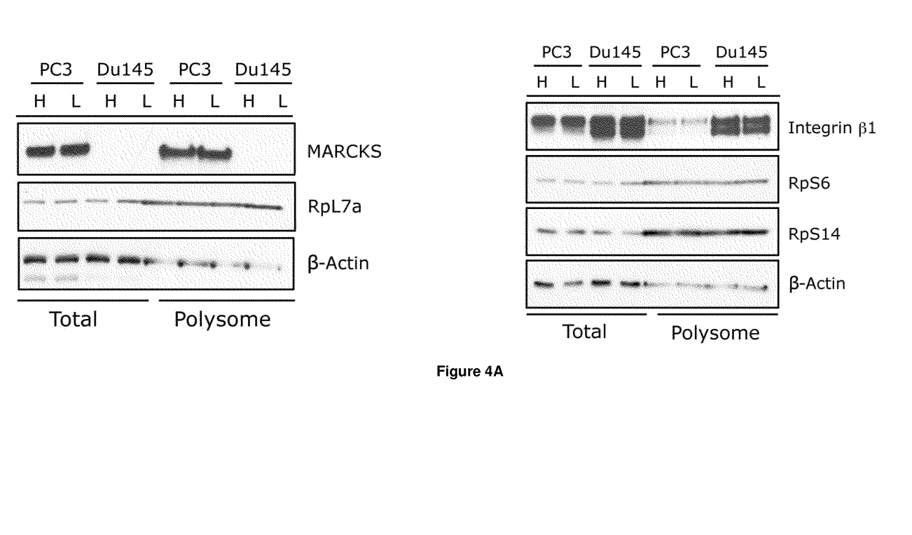

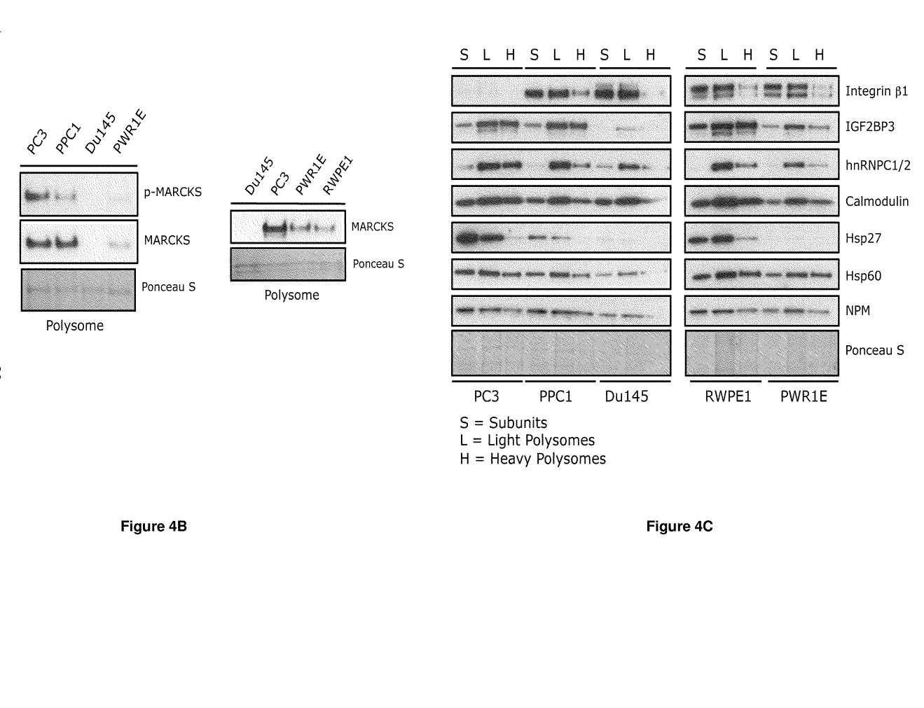

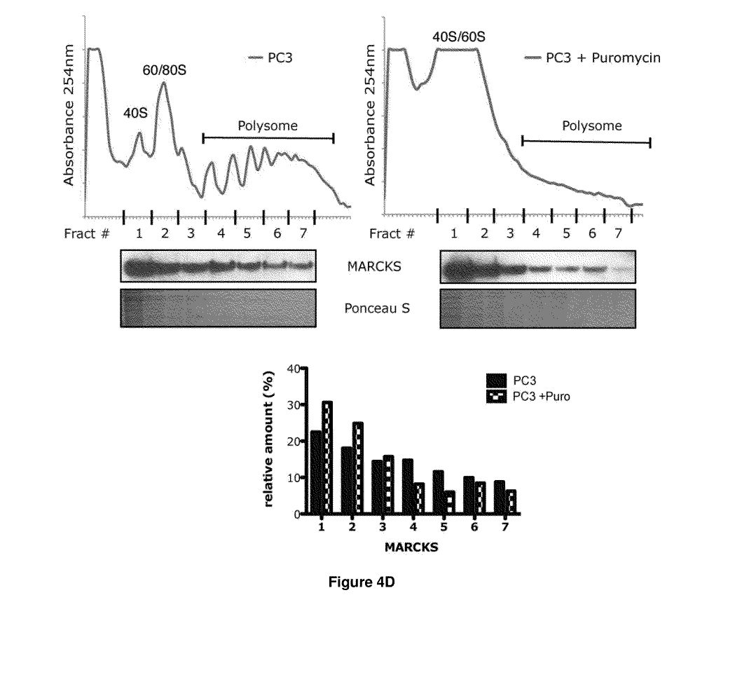

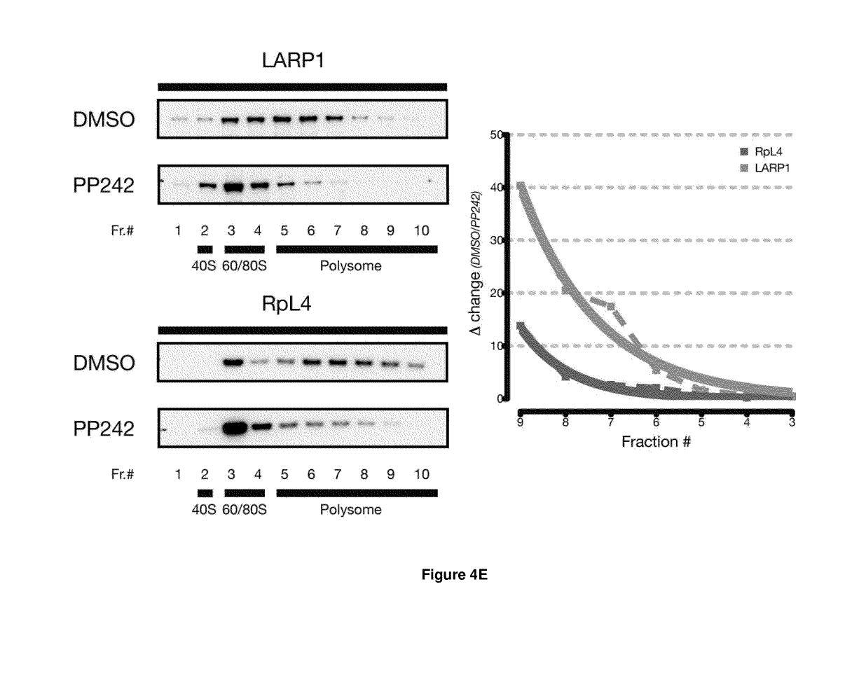

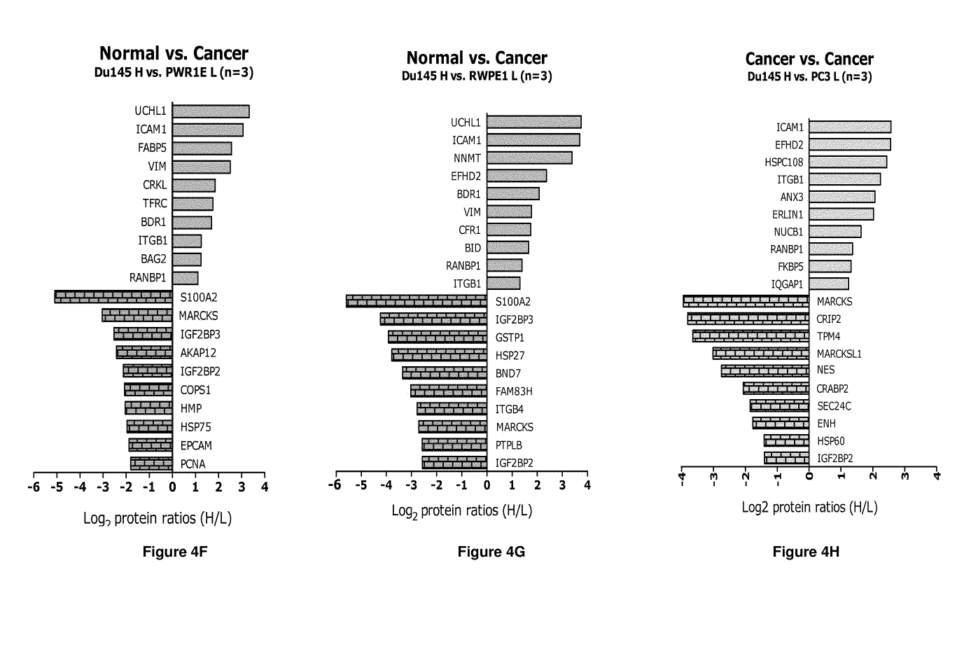

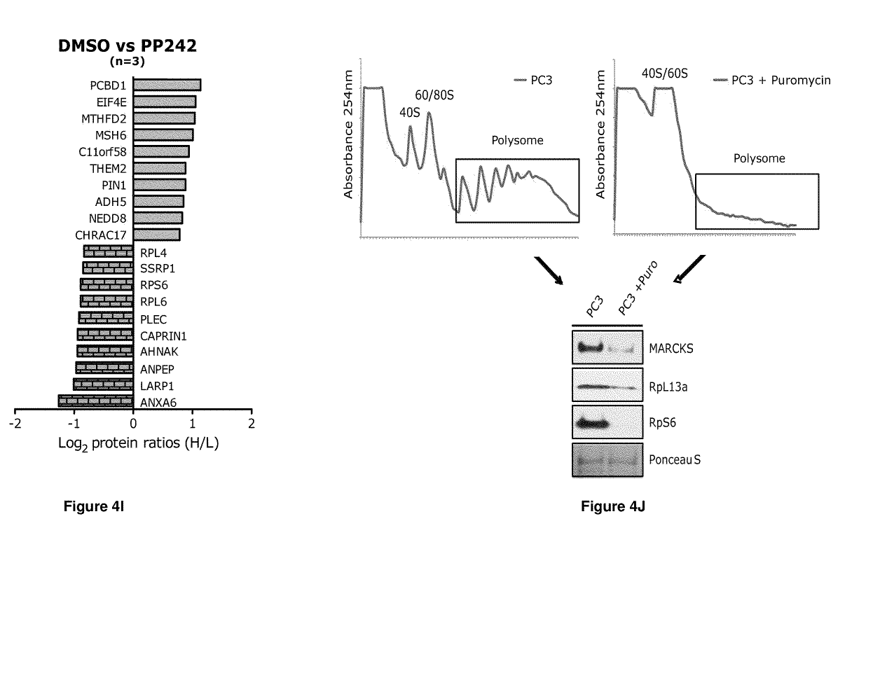

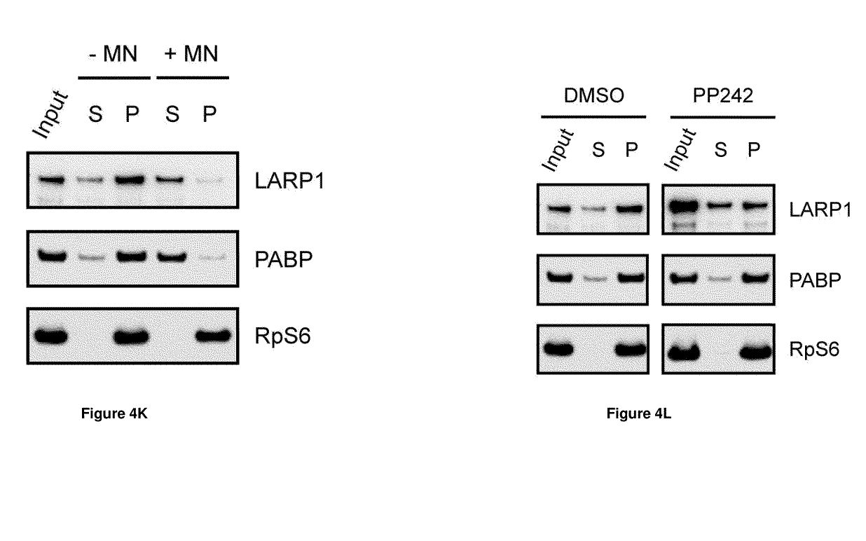

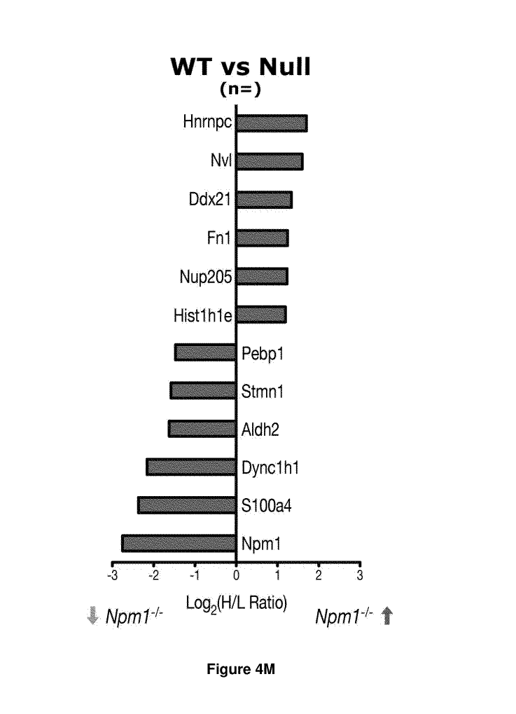

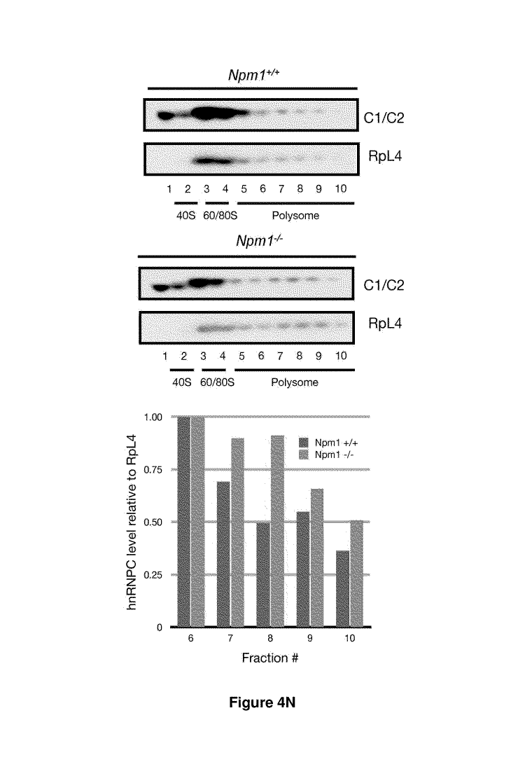

Cells were lysed in lysis buffer containing Complete Mini protease inhibitors (EDTA free) (Roche) and a Phosphatase Inhibitor cocktail (Thermo Scientific). 5-50 μg of total protein was subjected to SDS-PAGE on 4-12% Bis-Tris acrylamide NuPAGE gels in MOPS SDS running buffer (Invitrogen). The following primary antibodies were used: MARCKS, phospho-MARCKS (S152/156), Integrin β1, Calmodulin, Hsp27, Hsp60, RpL13a, RpL7a, and RpS6 (all Cell Signaling), HSP90, (BD Bioscience; BD Transduction Laboratories), hnRNPC1/C2 (Millipore), Rps14 and β-actin (all from Santa Cruz Biotechnologies). The NPM antibody was from DAKO and the IGF2BP3 antibody was from ProteinTech. Subsequently, membranes were incubated with secondary, HRP-tagged antibodies (Amersham) and signals were visualized with ECL or ECL plus (Amersham). It is important to note that Ponceau S staining was used as a control for equal protein loading in the western analysis of polysomal fractions, since typical house keeping genes like β-actin or α-tubulin are not enriched in riboproteome preparations and therefore only barely detectable in polysome fractions (FIG. 4A).

### Example 2

### High Throughput Analysis of the Riboproteome Using a SILAC-Based Approach

Without wishing to be bound by theory, the process of active translation within the cell may be regulated by a multitude of proteins that can interact with either the ribosome itself, the mRNAs that are being actively translated, or proteins that may have the capacity to interact with both the ribosome and mRNA.

In order to characterize the components that constitute the actively translating ribosome (i.e. the riboproteome) mass spectrometry was applied to quantitatively evaluate the protein components that are differentially associated with translation in different cellular contexts, while also allowing for a comprehensive overview of the proteins that make up the riboproteome.

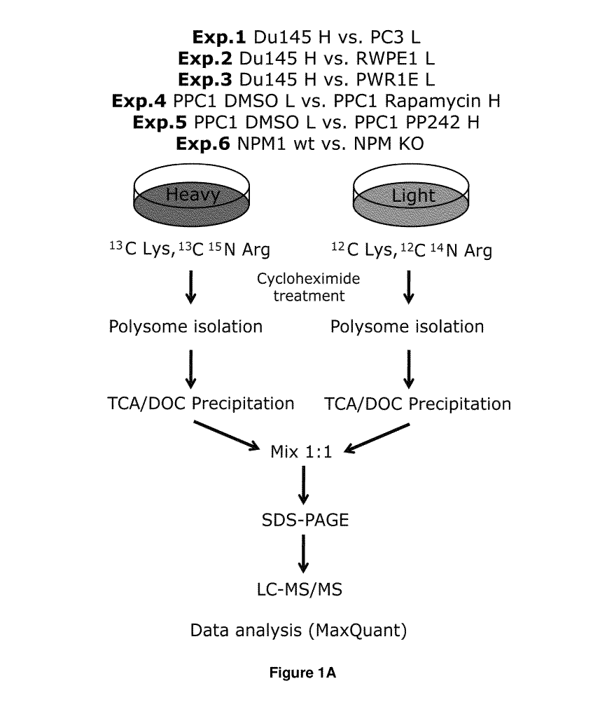

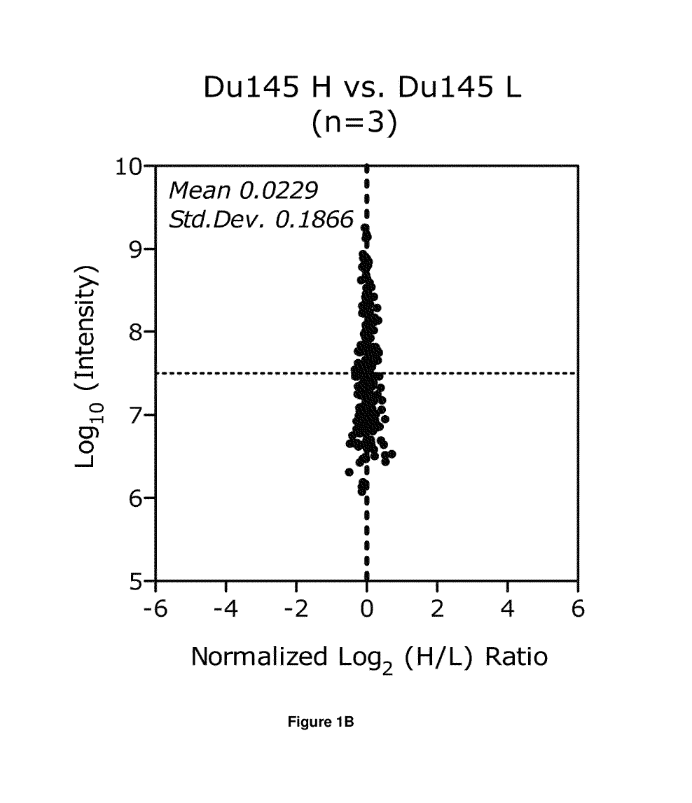

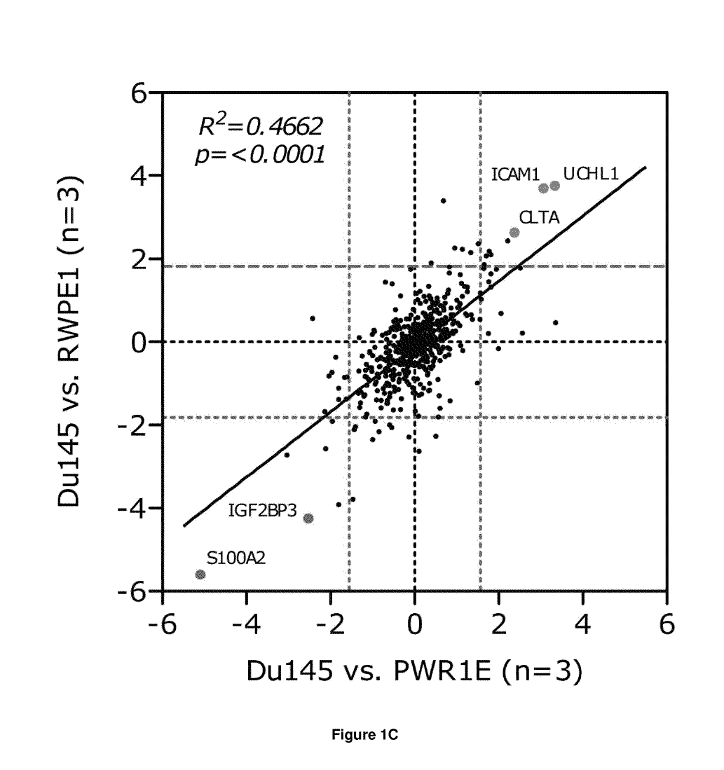

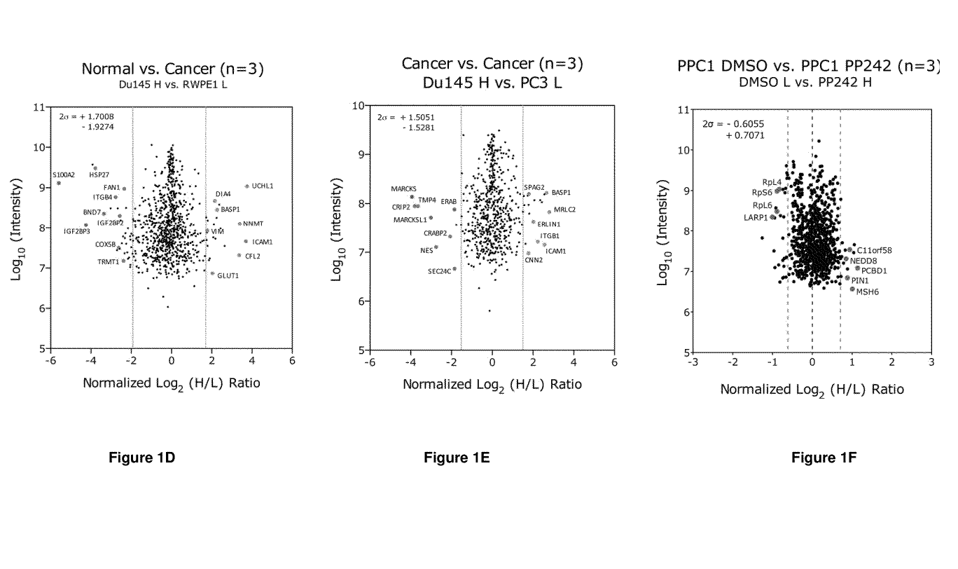

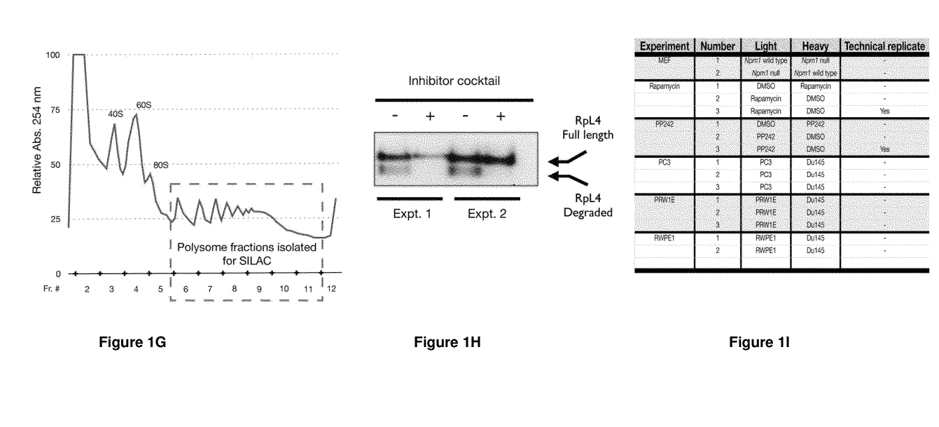

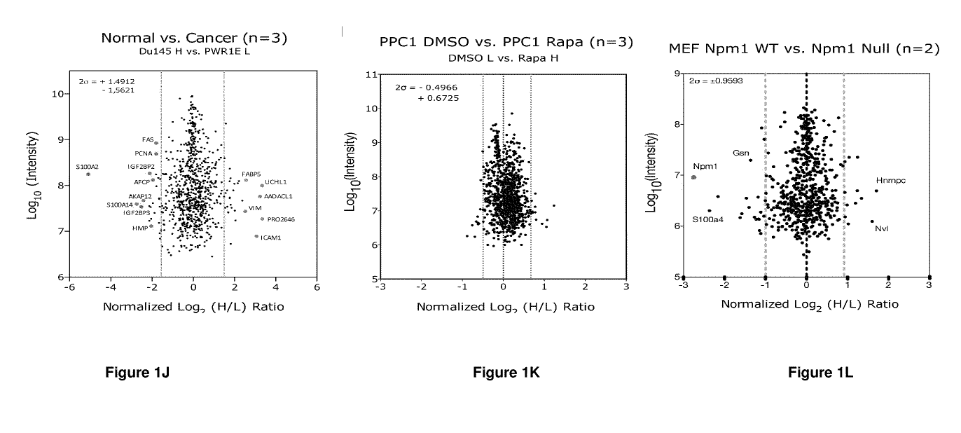

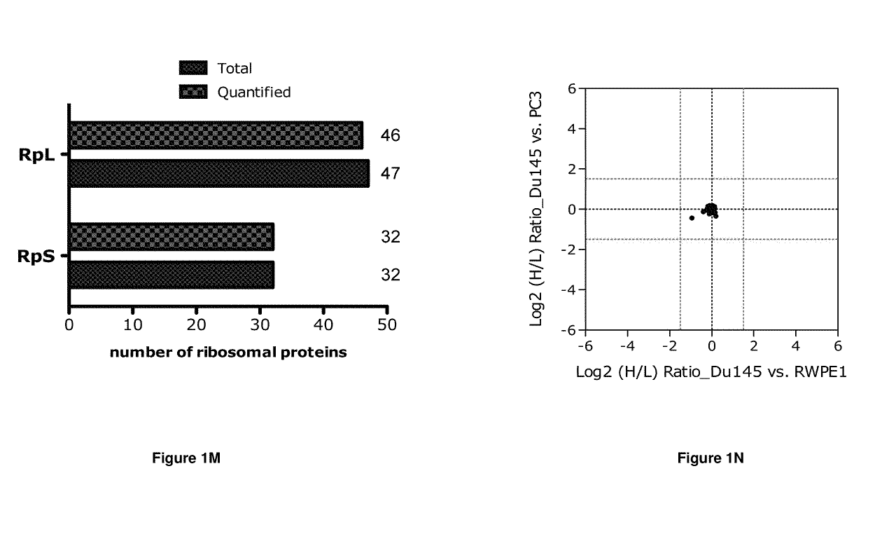

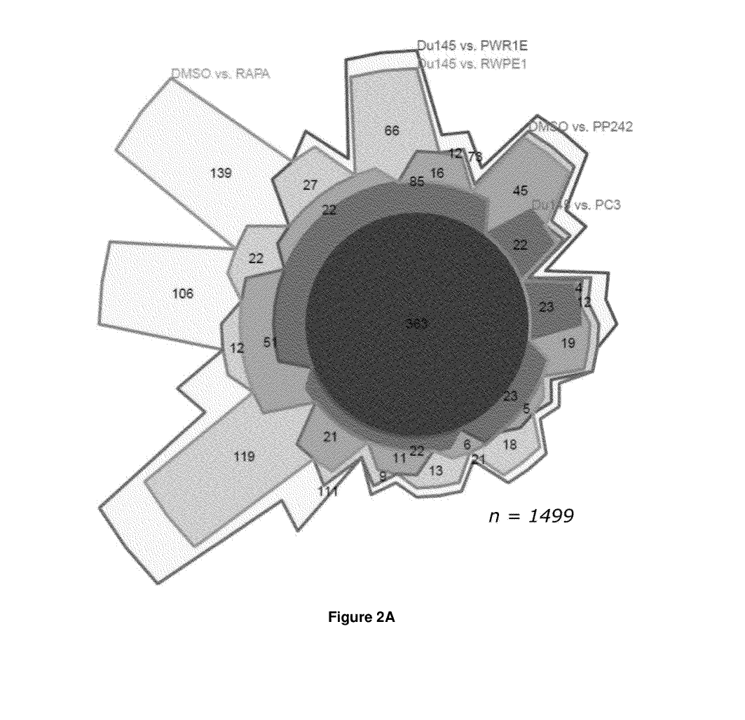

To this end, relevant cell lines of both mouse (e.g. mouse embryonic fibroblasts (MEFs)) or human origin (e.g. prostate cancer cell lines) were cultured with SILAC media to incorporate amino acids for Light (Lys₀^{C13}; Arg₀^{N14}) or Heavy (Lys₆^{C13}; Arg₁₀^{N15}) labeling of proteins and proceeded to isolate riboproteome components as outlined in FIG. 1A. Labeled cells were seeded to ensure sub-confluency at harvesting, and were treated with 100 μg/mL cycloheximide prior to harvesting (see Experimental Procedures). Cells were collected in PBS containing cycloheximide, and equal amounts of cell lysates loaded on 15%-50% sucrose gradients. Polysomes were separated by density gradient centrifugation, and collected by fractionation (FIG. 1G). Protein from individual polysome fractions was precipitated by deoxycholate-TCA precipitation, and resuspended in buffer (0.1M Tris pH 8.8; 1% SDS). Precipitated protein from fractions containing polysomes for Heavy and Light labeled cells were combined in a ratio of 1:1 (v/v) and run on an SDS-PAGE gel, which was subsequently stained using Coomassie Brilliant Blue. The gel lane was cut into 8 separate pieces and submitted for analysis by microcapillary liquid chromatography-tandem mass spectrometry (LC-MS/MS) analysis on a hybrid linear ion trap-orbitrap mass spectrometer. All resulting MS data were further processed with Mascot or Andromeda and the MaxQuant software suite as previously described in Cox et al., Nat. Biotechnol. 26:1367, 1372, 2008 and Cox et al., Proteome Res. 10:1794-1805, 2011.

This whole procedure required considerable optimization, as preliminary experiments identified extensive protease activity in polysomal fractions, resulting in degradation to ribosomal components, and affecting quality of mass spectrometry results (FIG. 1H). In order, to resolve these issues a comprehensive array of protease inhibitors was employed (as detailed in the Experimental Procedures section), which completely eliminated such protease activity and degradation artifacts.

To uncover the riboproteomic diversity within cellular populations, this approach was applied to a number of cell systems that included both relevant human prostate cell lines (Du145, PC3, PPC1 prostate cancer cell lines, and the immortalized prostatic epithelial cell lines PWR1E and RWPE1) and mouse embryonic fibroblast cells (MEF) (immortalized Npm1 wild-type and null) as outlined in FIGS. 1A and 1I.

Initially, actively translating polysomes from the normal prostatic epithelial cell lines PWR1 E and RWPE1 (two immortalized cell lines routinely used as normal controls for prostate cancer studies) were compared with the metastatic prostate cancer cell line Du145 (FIG. 1I). Secondly, the riboproteomes of the prostate cancer cell lines Du145 and PC3 were compared. The use of these four cell lines allowed the evaluation of how the riboproteome changes from a relatively normal situation (PWRE1, RWPE1) to a cancerous state (Du145) and between two different cancer cell lines that harbor distinct genetic alterations (Du145 PTEN^{wt}; TP53^{mut} and PC3 PTEN^{null}; TP53^{null}) (FIG. 1I). Thirdly, the riboproteomes of PPC1 prostate cancer cells (PTEN^{null}; TP53^{null}) treated with the mTOR inhibitors rapamycin and PP242 (FIG. 1I) were compared. Finally, MEFs harboring wild type or null alleles for the ribosome biogenesis gene Npm1 (FIG. 1I) were compared.

These data allowed, for the first time, determination of the overall composition of the ribosome and its associated proteins, and evaluation of quantitative differences in components of the mammalian riboproteome. Importantly, an initial comparison between polysomes derived from Du145 Heavy and Light labeled cells revealed that all quantified proteins showed an average Log₂ (H/L) ratio of around ‘0’ (226 quantified proteins; Mean 0.0029, STD±0.1866) (FIG. 1B) demonstrating that differences observed between cell lines do not arise from variations in sample preparation, and confirming both reliability as well as reproducibility of the technique.

In further support of this approach as a method to study composition and quantitative differences amongst riboproteomes, a comparison of the two normal and cancer cell line (hereafter referred to as N/C) datasets revealed a substantial overlap in identified proteins from immortalized normal epithelial cell lines, with a significant positive correlation (R²=0.4662, p=<0.0001). This demonstrates that these normal cell lines share significant similarity, which in turn gives greater significance to differences that exist between normal and cancer riboproteomes (FIG. 1C).

Importantly, several differences were detected between the riboproteomes of N/C datasets as well as between cancer cell types (hereafter referred to as C/C) using indicated cut-off values (cut-offs are based on two standard deviations from the mean) (FIGS. 1D, 1E and 1J). These differences are described in greater detail below, and include a variety of proteins including RNA binding proteins (RBPs) (e.g. IGF2BP2, IGF2BP3), cell adhesion molecules (e.g. Integrin β1), and signaling proteins (e.g. MARCKS) amongst others.

In addition, acute exposure to the mTOR inhibitors rapamycin and PP242 in PPC1 cells reveals that only strong inhibition of the mTOR kinase itself results in a clear perturbation to the riboproteome (FIGS. 1F and 1K). This is consistent with the differential capacity of these drugs to inhibit mTOR activity towards translation (DMSO<rapamycin<PP242) with numerous ribosomal proteins and RBPs (e.g. RpL4, RpL6, RpS6, LARP proteins) demonstrating the most striking quantitative differences (FIGS. 1F and 1K).

Comparing Npm1 wild type or null immortalized MEFs, mass spectrometry data from two separate biological replicates were combined, including a label switch. No change in relative quantification of ribosomal proteins was observed between Npm1 wild type and null immortalized MEFs (FIG. 1L). Interestingly, Npm1 was identified as the most highly decreased protein in Npm1 null riboproteomes (FIG. 1L), due to the presence of N-terminal peptides that remain as a result of the knockout strategy, thereby serving as an internal positive control.

Notably, the approach identified and quantified all but one (RpL41, a lysine and arginine rich 25 amino acid protein that is unlikely to be identified by this mass spectrometry approach due to the large number of sites available for trypsin cleavage, and the consequent inability to generate multiple peptides) ribosomal proteins (FIG. 1M), as well as other known translation-associated proteins including initiation and elongation factors. It was observed that ribosomal proteins of both the small and the large subunit cluster around a normalized Log₂ (H/L)=0 (FIG. 1N), indicating that ribosomal proteins are unchanged between normal and cancer cells, as well as between cancer cell lines and genetically defined MEFs. These data make the important point that, at least amongst these cell lines, core ribosomal protein composition in polysomes is not altered.

### Example 3

### Characterization of the Riboproteome

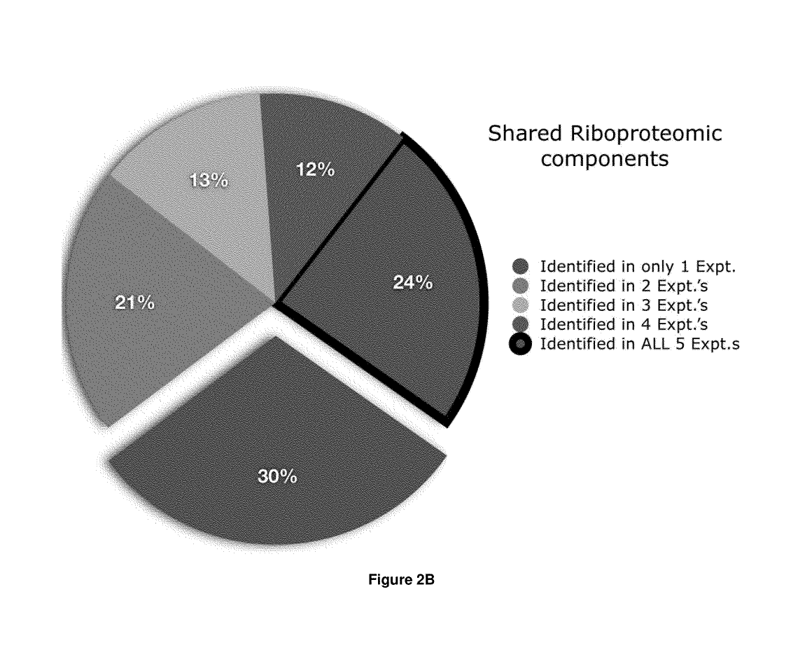

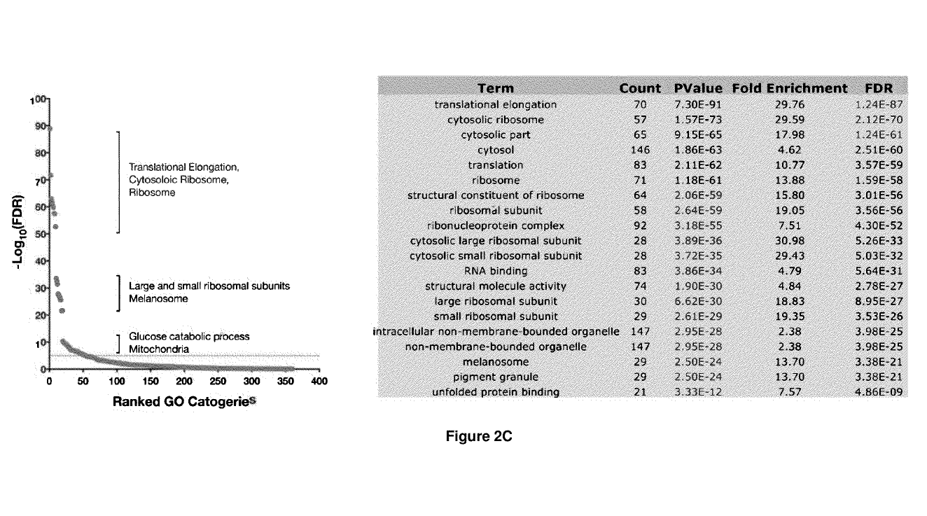

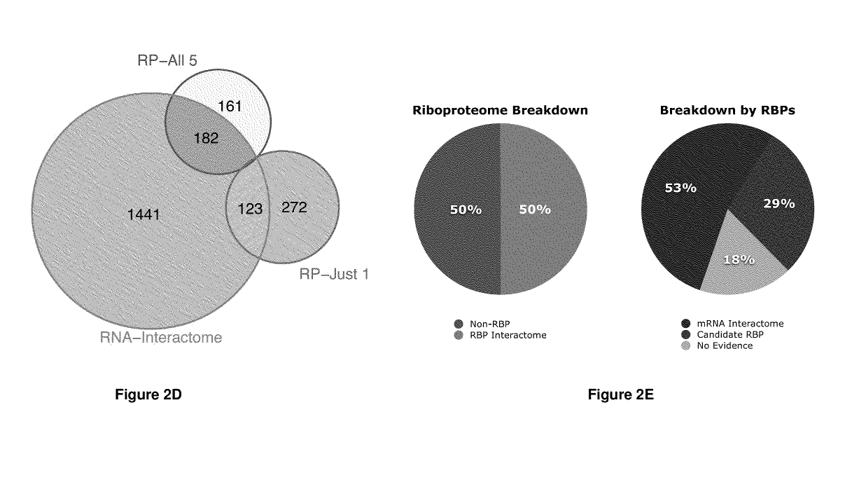

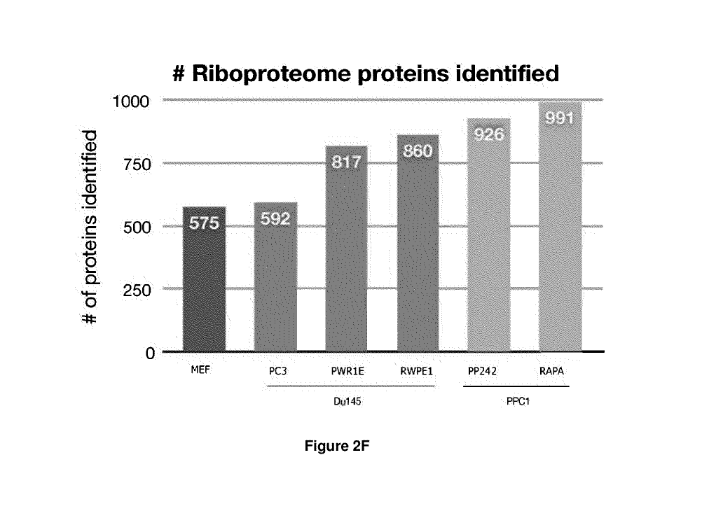

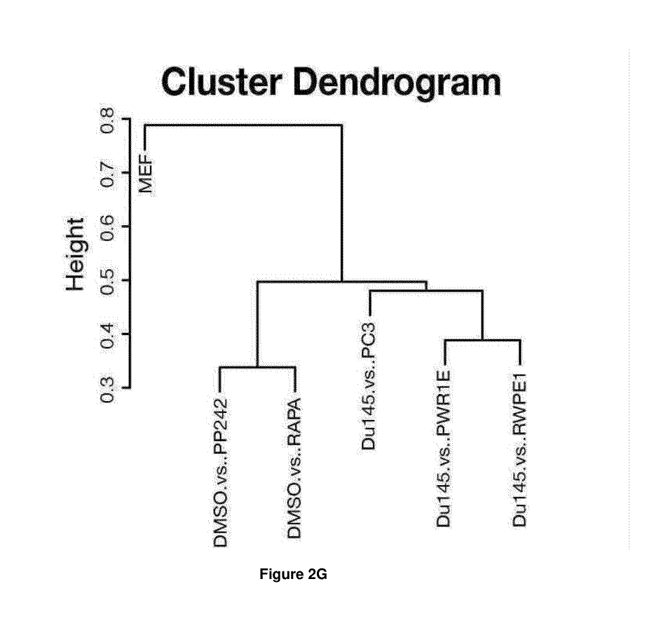

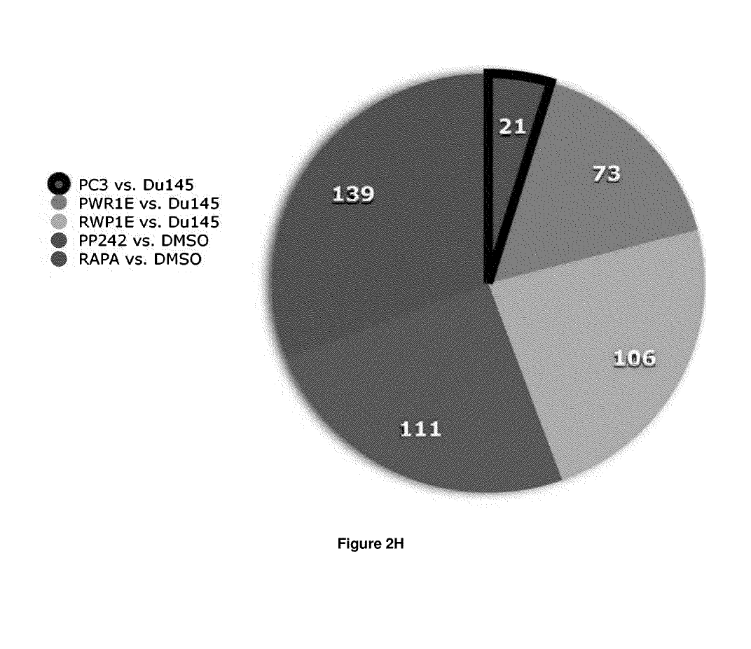

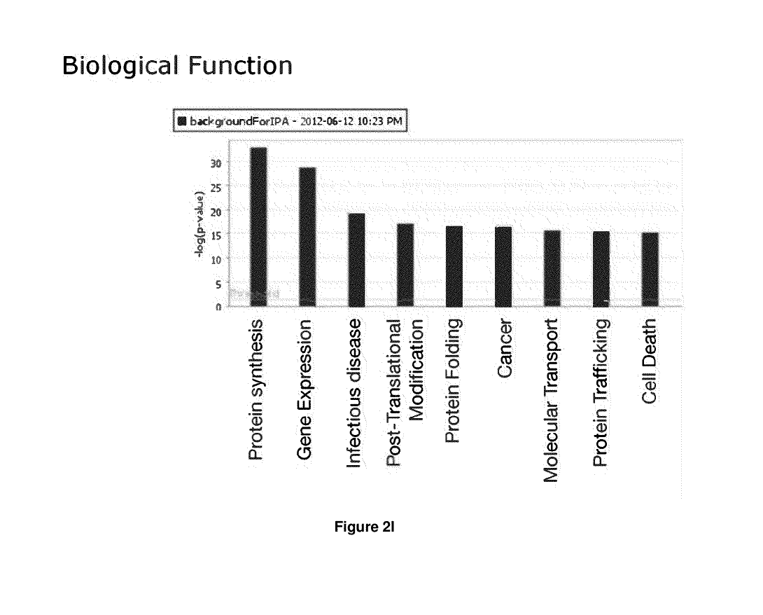

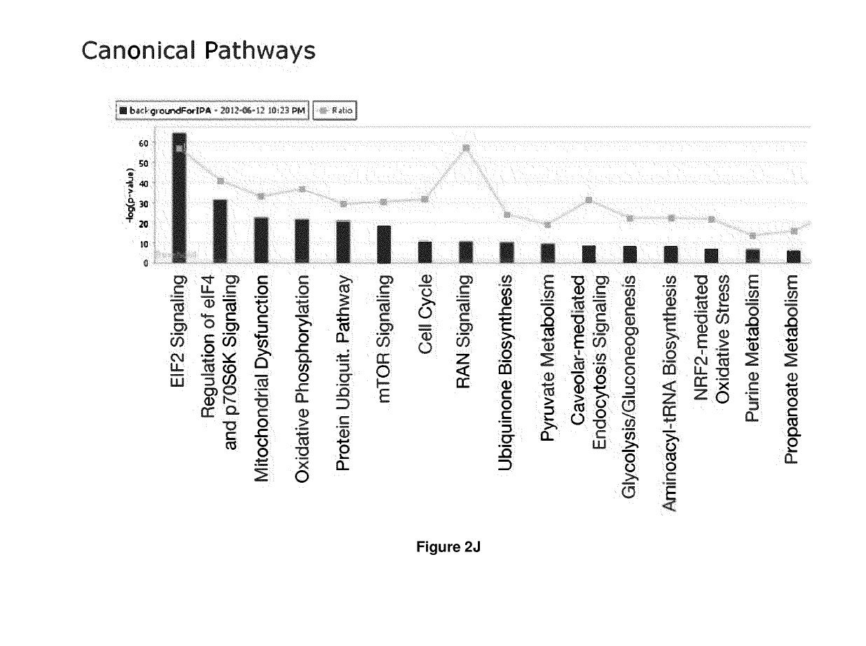

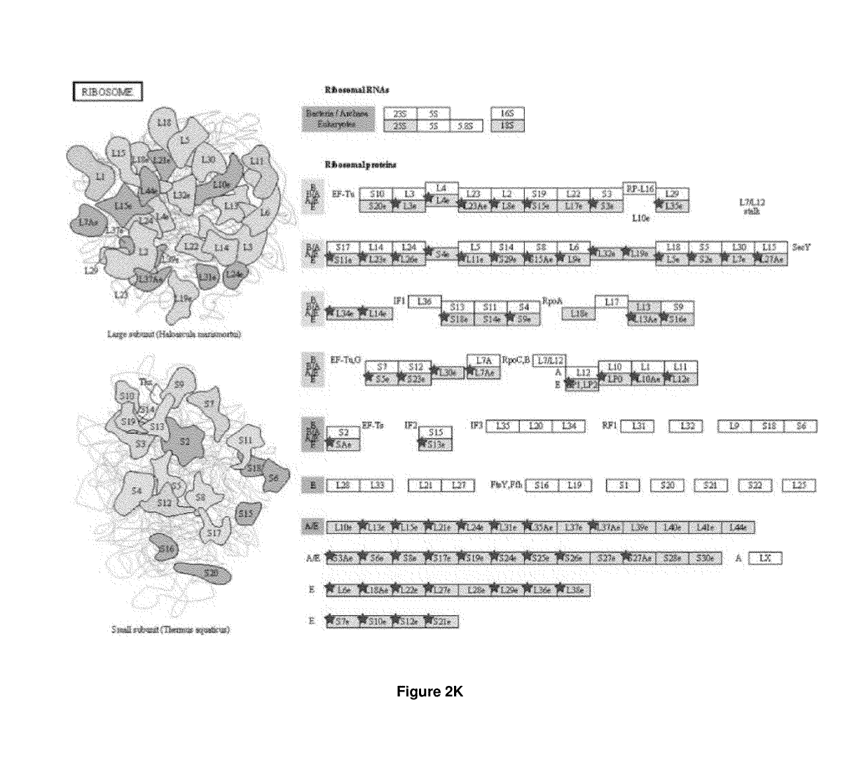

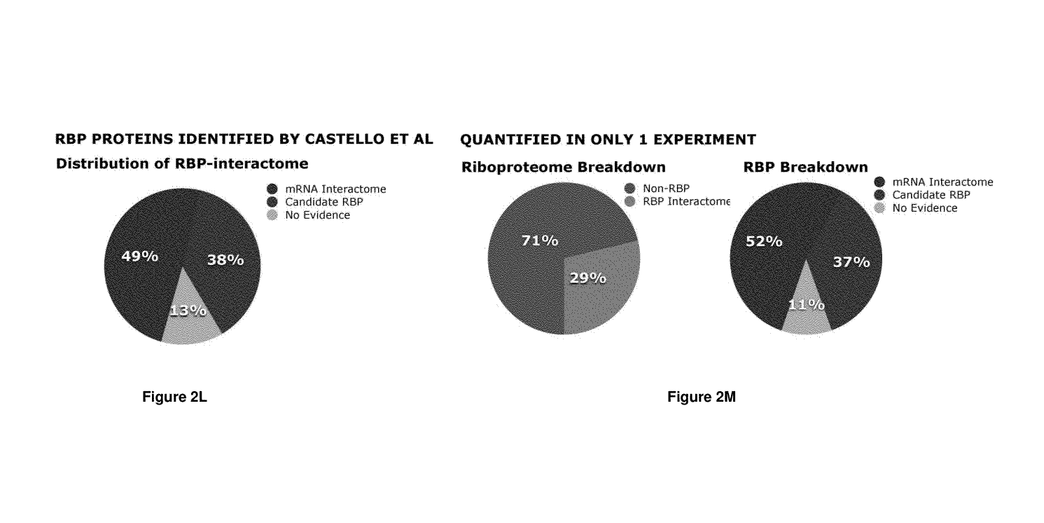

Overall, the number of proteins quantified in each of the individual groups of experiments varied from 575 to 991 (FIG. 2F), and offered the potential to uncover significant overlap of proteins that make up the riboproteomic space in mammalian cells.

To first examine how the datasets compared to one another an unsupervised hierarchical clustering of the 6 conditions was analyzed (FIG. 2G). Interestingly, the MEF dataset appeared to cluster independently from the human prostate cancer cells, while amongst the prostate cancer cells, PPC1 cell lines cluster together and the immortalized prostate epithelial cell lines cluster together. The PC3 and Du145 experiments displayed greater similarity to immortalized epithelial cells, likely due to their shared comparison. These data indicate that the riboproteome itself may have the capacity to categorize cell types and tissues based on riboproteomic diversity, and in turn can contribute to regulation of gene expression within a given cellular compartment.

In addition to a number of significant differences identified between the various samples, the hierarchical clustering clearly demonstrated all prostate cell lines shared high similarity. Thus, these data sets were combined in order to gain a global perspective of the prostate riboproteome. In the combined prostate cell line dataset, a total of 1499 quantified proteins was identified (FIG. 2A). Of these 1499 proteins, 70% were identified in at least two experimental datasets, while 24% (363 of 1499) were identified in all 5 experiments (FIG. 2B). Indeed, this number of 363 core riboproteomic components represents over 60% of the PC3/Du145 SILAC experiment, which contained the lowest number of proteins identified in the prostate cell line cohort (FIG. 2F). It is also interesting to note that 96% of proteins quantified in this PC3/Du145 dataset were found in at least one other dataset, with only 21 proteins quantified unique to this experiment. These data show strong overlap in proteins identified amongst the independent riboproteome experiments, and highlights the advantage of using multiple cell lines to characterize the riboproteome.

Ingenuity Pathway Analysis (http://www.ingenuity.com/) of all 1499 proteins identified was carried out to examine what (1) biological functions and (2) canonical pathways may be specifically enriched in the dataset. Importantly, biological functions related to protein synthesis, post-translational modification and protein folding were found to be highly enriched in our combined dataset (FIG. 2I). In agreement with this, the canonical pathway analysis demonstrated EIF2 signaling, regulation of eIF4 and p70S6K signaling, and mTOR signaling pathways to be significantly represented (FIG. 2J).

In addition, KEGG pathway analysis of proteins identified in all 5 experimental datasets (363/1499) compared to proteins identified in at least 1 experiment (1499) identified ribosome related pathways to be highly enriched (FIG. 2K).

### Example 4

### Diversity of Protein Functional Groups and Enrichment of RNA Binding Proteins in the Riboproteome

To better understand the various protein components that make up the riboproteome, DAVID (david.abcc.ncifcrf.gov) (Huang et al., Nat Protoc 4:44-57, 2009) was used to perform a gene ontology-based functional categorization of the proteins identified in the combined dataset (FIG. 2C). This analysis demonstrated a clear and significant enrichment in ribosome and translation related processes. In addition, a number of other diverse protein functional groups were found to be included in the riboproteome, including melanosome and glucose catabolic processes. Critically, a significant enrichment of RBPs to be constituents of the riboproteome were identified. As two recent papers published now describe the RNA-binding protein interactome in detail (Baltz et al., Mol Cell 46:674-690, 2012; Castello et al., Proteins Cell 149:1393-1406, 2012), the riboproteome and RBP-interactome datasets were compared to evaluate the proportion of RBPs that form part of the riboproteome. Using the dataset from Castello et al. a considerable overlap between the RBP-interactome and riboproteome (FIG. 2D) was found. Strikingly, core riboproteome components show themselves to be enriched in RBP-interactome proteins (50% of proteins identified can be assigned to the RBP-interactome, FIG. 2E, left panel), while those proteins identified in only one experimental condition have a much lower RBP-interactome component (only 29% of proteins identified can be assigned to the RBP-interactome, FIG. 2M, left panel). Moreover, it is interesting to note that when the riboproteome RBPs were broken down according to the categories defined by Castello et al. in FIG. 2L (i.e. mRNA-Interactome; Candidate RBP; No Evidence), the proportional distribution in these three categories is highly similar to those described by Castello et al. (FIGS. 2E and 2M, right panels for RBP categories from the riboproteome).

### Example 5

### Riboproteomic Genes are Frequently Amplified in Human Cancer

As cellular proliferation is strongly coupled to translation, the riboproteome was evaluated to see if it may be altered in human cancer using the cBio Cancer Genomics Portal (http://cbioportal.org) and the R package cgdsr, developed at Memorial Sloan-Kettering Cancer Center.

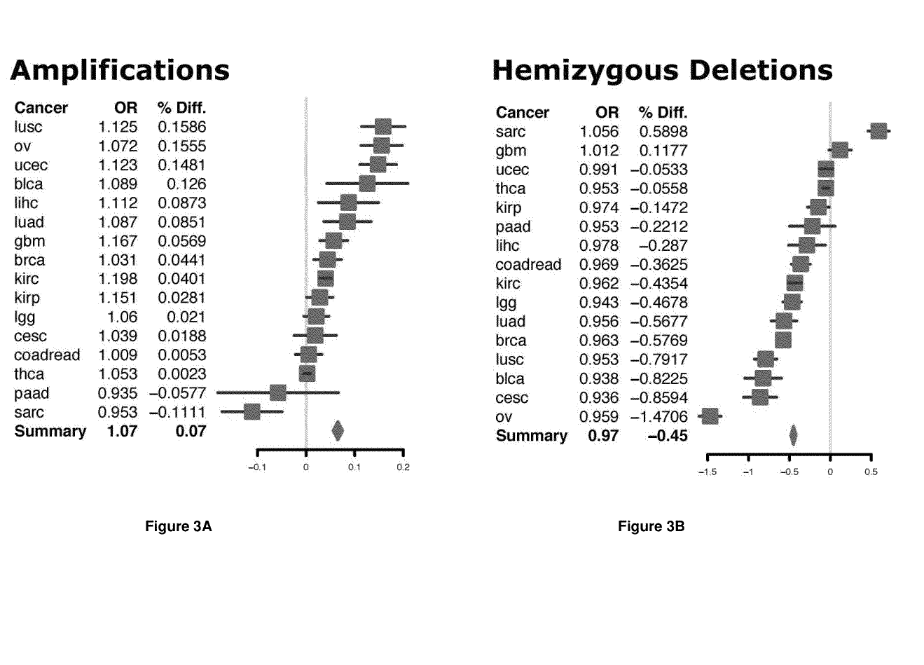

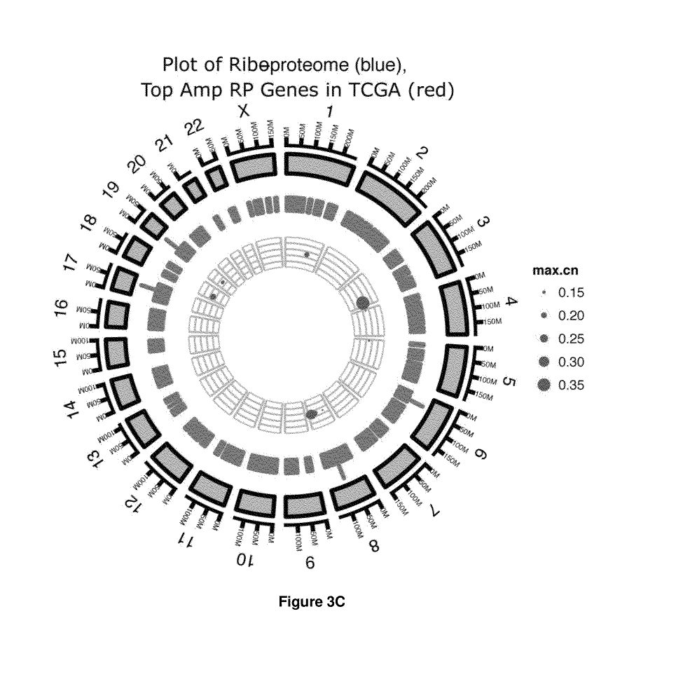

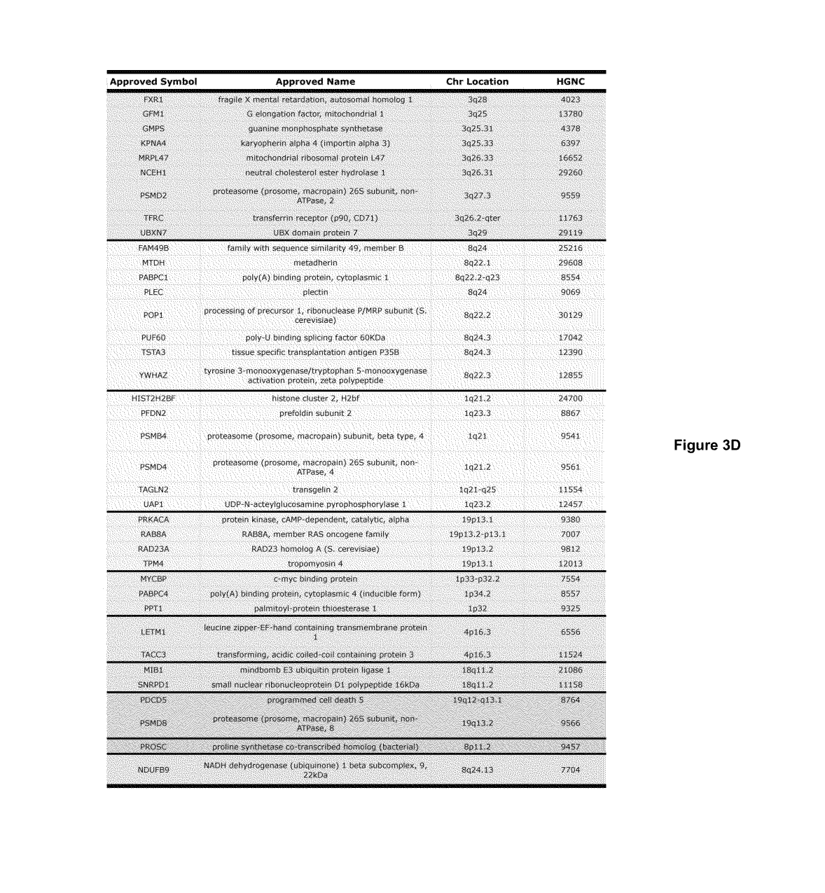

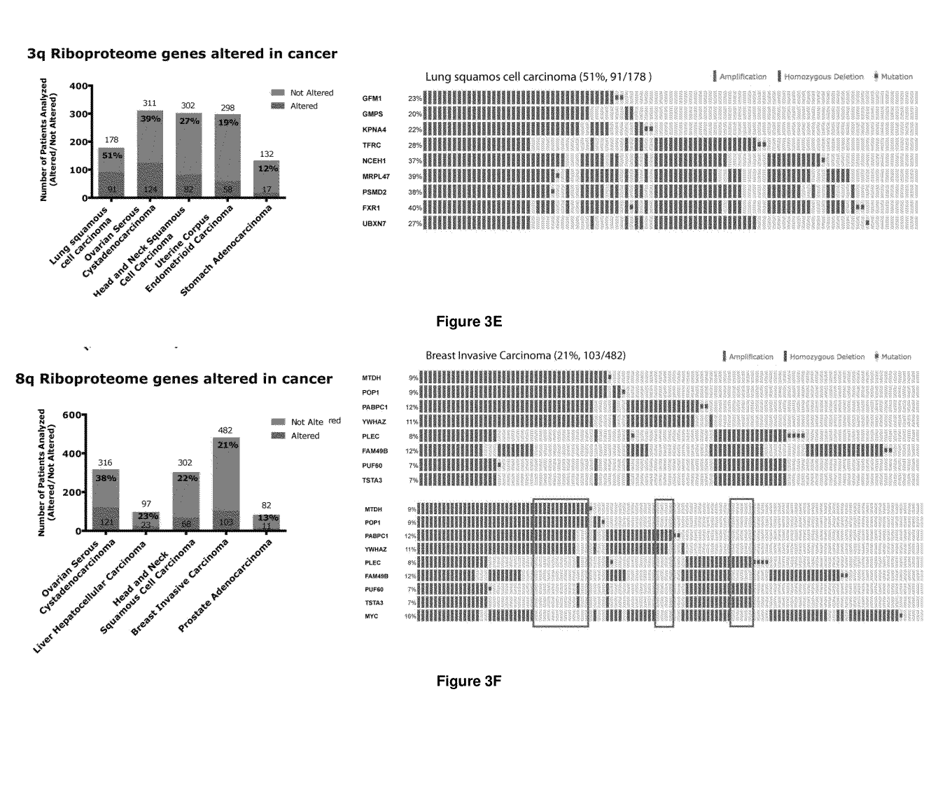

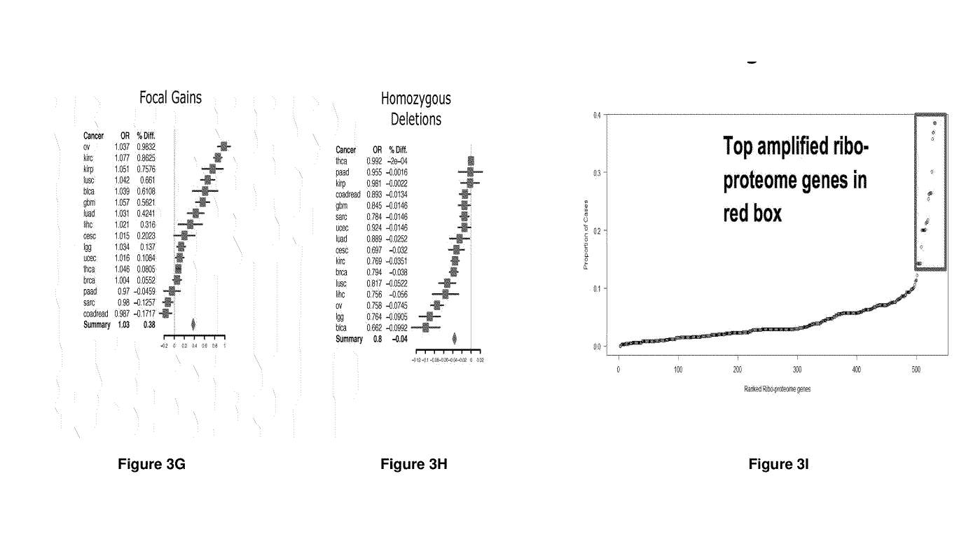

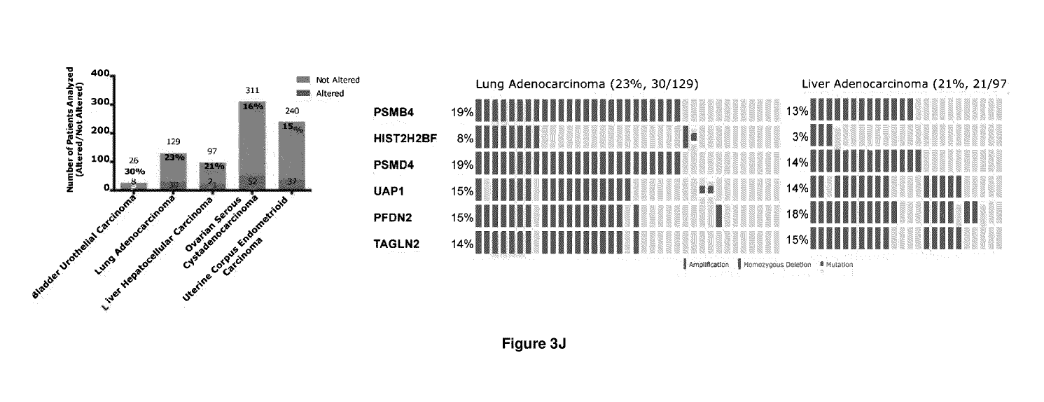

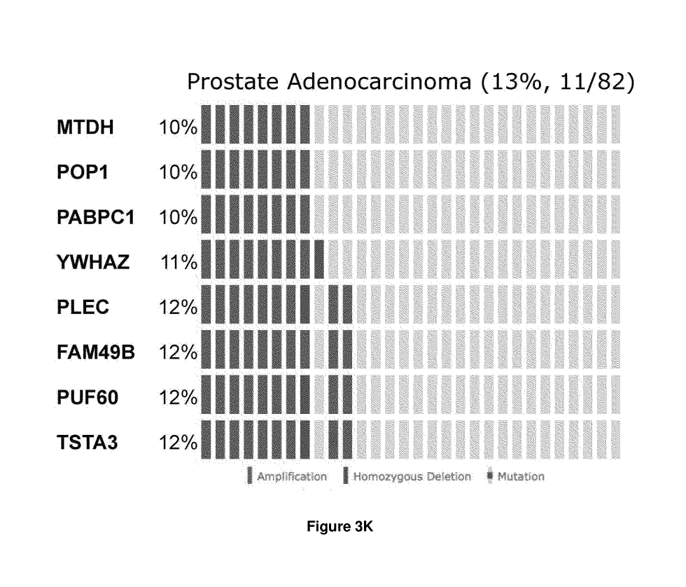

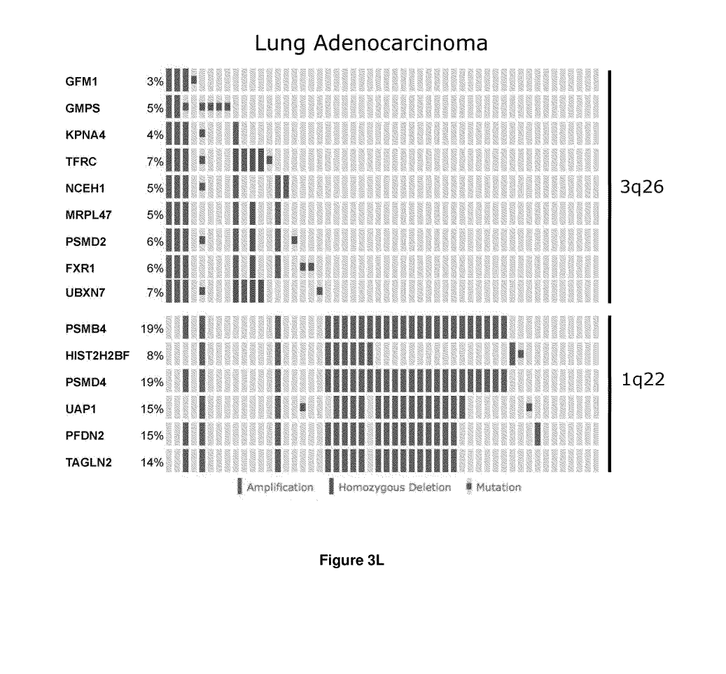

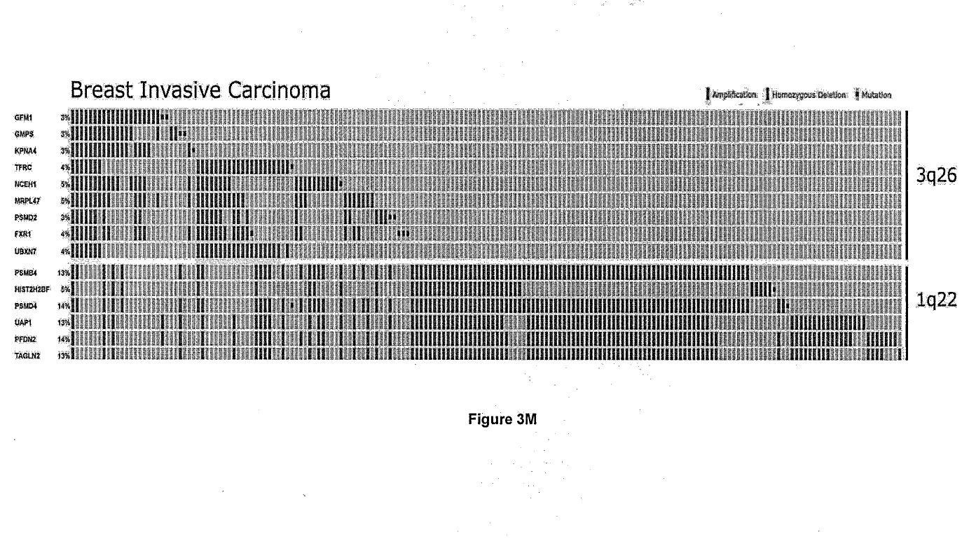

The distribution of copy number alterations in the riboproteome was examined as compared with genes in the background genome across 16 cancer types. This analysis included between 1661 and 1720 of the riboproteome genes (median=1675) and 19195 non-riboproteome background genes. The overall analysis shows that the riboproteome is enriched for copy-number gains and high-level amplifications (FIGS. 3A and 3G) and depleted for hemizygous and homozygous deletions (all P<2.2e⁻¹⁶) (FIGS. 3B and 3H).

Based on these data, we sought to identify riboproteomic genes that undergo the most frequent CNA's in specific cancer types. This analysis focused on 532 riboproteome genes with complete copy number data across 15 cancer types. Riboproteome genes were ranked by the maximum number of cases where they showed a genomic amplification across the 15 cancers (FIGS. 3C and 3I). Interestingly, 38 riboproteome genes correlated with high-level amplifications in at least 10% of at least 1 cancer type was observed (FIG. 3D). While several genetic loci are represented in this data set including 4p16.3, 1p33 and 19p13, more than half of these genes (60%, 23 of 38) grouped to three specific genetic loci. These loci represented 1q22, 3q26 and 8q24, with regions surrounding chromosome 3q26 and 8q24 identified as showing most frequent amplification (FIGS. 3C and 3D). Analysis of the gene signature for the 3q riboproteomic gene locus (9 genes) in the TCGA studies containing mutation data showed that 51% (91 of 178 cases) of lung squamous cell carcinoma contained an alteration in at least one of these genes (FIG. 3E, left panel). Ovarian serous cyst adenocarcinoma showed alterations of 39% (FIG. 3E, left panel), while patient samples from other cancer types also showed alterations in this 3q gene set (FIG. 3E, left panel). Analysis of the same datasets for the 8q riboproteome gene locus, identified breast invasive carcinoma to harbor frequent alterations to genes in this locus (21% of cases, 103 of 482 cases) (FIG. 3F, left panel). Ovarian serous cyst adenocarcinoma patients also showed significant alteration (38%%, 121 of 316 cases), while prostate cancer patients showed alteration in 13% of patients at this locus (11 of 82 cases (FIGS. 3F, left panel and 3K)). Accordingly, closer analysis of 3q26 and 8q24 riboproteome gene groups in individual patients clearly demonstrate frequent co-amplification of these genes, in line with our hypothesis that riboproteomic genes are preferentially amplified in cancer (FIGS. 3E and 3F, right panels). Similarly, the 1q22 locus demonstrates a frequent amplification in various cancer types (FIG. 3J).

Interestingly, both 3q26 and 8q24 harbor established oncogenes, PIK3CA and MYC respectively. While it may be considered that the riboproteomic genes in these regions may be simply amplified along with the dominant oncogene at the relevant locus, our cBio analysis clearly identifies a number of patients with invasive breast carcinoma without MYC amplification or mutation, while still harboring amplification of 8q24 riboproteome genes (FIG. 3F, right panel), suggesting that they have the potential to promote tumorigenesis independent of MYC.

Furthermore, the amplified riboproteomic loci were infrequently co-amplified in a number of cancer types. For example, limited co-occurrence of 3q26 and 1q22 amplification is observed in patients from lung adenocarcinoma and breast invasive carcinoma cancer datasets (FIGS. 3L and 3M).

### Example 6

### The Riboproteomic Platform for the Identification of Riboproteomic Components and Regulators of Translation

The differences in N/C cells as well as C/C cells were the next focus to identify riboproteomic components and as a means to validate the approach. As mentioned above, the datasets revealed marked differences in proteins quantified between polysomal fractions of normal and cancer cells (i.e. RWPE1 and PWR1E cells compared to Du145 cells), indicating that the Du145 cancer cells display numerous differences in the composition of their riboproteome (FIGS. 1D and 1J). These differences encompass a variety of protein types and include a number of potential ribosome-associated proteins that were reproducibly enriched on the polyribosomes of either normal or cancer cells, including intracellular adhesion molecule 1 (ICAM1), vimentin (VIM) and integrin β1 (ITGB1) that are enriched in cancer cells as well as the RBPs IGF2BP2 and IGF2BP3 that were amongst others reproducibly enriched in normal cells (FIGS. 4F and 4G). In order to further establish the relevance of the riboproteome in the context of cancer, we focused on differentially quantified proteins associated with polyribosomes of prostate cancer cells (i.e. PC3 cells compared with Du145 cells) (FIGS. 1E and 4H).

Amongst these differentially quantified proteins MARCKS stood out as it was a highly differential factor (FIG. 1E and S4C), and a major cellular substrate for Protein Kinase C (PKC), suggesting that MARCKS might represent a regulator of cellular translation and a candidate for further validation.

Importantly, MARCKS was strongly associated with polyribosomes of the prostate cancer cell line PC3 when compared to Du145 cells (FIGS. 4A and 4B). To further confirm this observation, polysomal fractions from three prostate cancer cell lines (PC3, PPC1 and Du145) were isolated as well as the two normal immortalized-epithelial control cell lines (PWR1E, RWPE1) and subjected pooled polysomal fractions to western blot analysis. Indeed, it was confirmed that PC3 and PPC1 cells displayed increased amounts of MARCKS on polyribosomes when compared to either Du145 or prostatic epithelial cell lines (FIG. 4B). In addition, these cells also displayed high levels of phosphorylation of the PKC sensitive serine residues of MARCKS (S159 and S163) (FIG. 4B). This analysis also validated the SILAC findings that ribosomal protein levels (e.g. RpS6, RpS14 and RpL7a) remain unaltered between cancer cell lines (FIG. 4A). In contrast to MARCKS, the SILAC analysis revealed that integrin β1 was highly enriched in Du145 cells when compared to PC3 cells, and western blot analysis also confirmed this differential enrichment (FIG. 4A).

To extend this validation and analysis further, additional western blot analysis was carried out on lysates from each of the prostate cell lines utilized in this screening. As expected, the presence of all proteins analyzed in the polysomal fractions collected were confirmed (FIG. 4C). Additionally, these data confirm the SILAC predictions regarding differential expression of proteins (e.g. compare integrin β1 in PC3 and Du145 lysates, or IGF2BP3 in Du145 and RWPE1 lysates, FIG. 4C).

As an additional validation for polysomal association, a well-established method of puromycin-mediated dissociation of ribosome-mRNA complexes was employed (Blobel and Sabatini, Proc Natl Acad Sci USA 68:390-394, 1971). As shown in FIG. 4J, puromycin treatment results in the loss of RpS6 and RpL13a from polyribosomes as determined by western blot analysis of pooled polysomal fractions. As an example of a riboproteome component associating with polyribosomes, the presence of MARCKS was also dramatically decreased in polyribosome fractions upon puromycin treatment (FIGS. 4D and 4J), which in addition supports the hypothesis that MARCKS plays a role in translation through association with actively translating ribosomes.

It was hypothesized that the riboproteomic platform would allow for the identification of mechanisms of response to pharmacological perturbation and for translational targets that could be differentially exploited for therapeutic intervention in cancer.

To this end, riboproteomic analysis upon inhibition of mTOR using the inhibitors rapamycin (a TORC1 inhibitor (Thoreen and Sabatini, Autophagy 5:725-726, 2009)) and PP242 (a mTOR kinase inhibitor that inhibits TORC1 and TORC2 activity simultaneously (Feldman et al., PLoS Biol 7:e38, 2009)) was performed as mentioned above. This analysis revealed that the riboproteome is indeed differentially responsive to treatment modalities. Although, it was found that rapamycin has little impact on the composition of the riboproteome (FIG. 1K), the more potent mTOR kinase inhibitor PP242 results in a much stronger and more robust perturbation of the riboproteome (FIG. 1F). Indeed, while inhibition of mTOR by PP242 identifies a number of proteins, including some ribosomal proteins (e.g. see RpL4, RpL6 and RpS6 in FIG. 4I), that show a rapid and significant disassociation from the riboproteome upon treatment with PP242, this may represent a more general dissociation of the ribosome and a block in translation. Interestingly, the RBP LARP1 (La ribonucleoprotein domain family member 1) appeared to be one of the most dynamic components of the riboproteome in response to mTOR inhibition by PP242 (FIGS. 1F and 4E). Although the function of LARP1 is not completely understood, it has been reported to play a role in cell division, apoptosis and migration (Burrows et al., Nucleic Acids Res. 38: 5542-5553, 2010), and it has been shown to be an mTOR sensitive phosphoprotein (Hsu et al., Science 332:1317-1322, 2011; Yu et al., Science 332:1322-1326, 2011). The RNA binding activity of LARP1 was confirmed (Burrows et al., Nucleic Acids Res. 38:5542-5553, 2010), by using a micrococcal nuclease (MN) assay (Darnell et al., Cell 146:247-261, 2011). Pooled sucrose gradient fractions containing LARP1 protein, were treated with and without MN. Ribosomes were subsequently pelleted by ultracentrifugation, and the protein in supernatant and pellet isolated for western blot analysis. As seen in FIG. 4K, LARP1 behaved similar to the well-characterized poly-A binding protein (PABP). Without MN treatment, LARP1 pelleted with ribosomal proteins, indicating its close association with polysome components. However, upon treatment with MN, LARP1 no longer associated with riboproteome components, and is released into the supernatant similar to PABP (FIG. 4K). This indicates that LARP1 is predominantly an RBP, showing limited association with the ribosome itself. In addition, treating cells with PP242 prior to this analysis, it was observed that while PABP appears to remain tightly intact with polysome fractions, there appears to be more LARP1 observed in the supernatant, suggesting that mTOR inhibition can selectively influence binding of LARP1 at the polysome (FIG. 4L). Thus, these findings suggest that mTOR activity towards LARP1 may represent an additional means by which mTOR can regulate translation.

Finally, to examine whether the riboproteome is altered in response to a genetic perturbation, SILAC riboproteomic analysis on Npm1 wild type and null immortalized MEF was analyzed (immortalized by deletion of the Trp53 gene) (FIG. 1L). Again, SILAC analysis of polysome fractions demonstrated a high similarity between riboproteome components, with ribosomal proteins themselves showing no quantitative difference between the Npm1 wild type or null MEF preparations (FIG. 1L). However, there were a number of proteins that demonstrated differential association with polysomes from Npm1 wild type and null MEFs, which may be relevant for translation in these cells (FIGS. 1L and 4M). Interestingly, the heterogeneous nuclear ribonucleoprotein hnRNPC was identified to be one of the most highly increased proteins on the polysomes of Npm1 null MEFs (FIG. 4N).

Thus, taken together these data validate this approach as an effective means to study riboproteome composition in a wide variety of cellular contexts, and highlight this approach as a valuable resource that can be applied to the study of how perturbations to genes and pathways impact the riboproteome.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure come within known customary practice within the art to which the invention pertains and may be applied to the essential features herein before set forth.

All publications, patents, and patent applications are herein incorporated by reference in their entirety to the same extent as if each individual publication, patent or patent application was specifically and individually indicated to be incorporated by reference in its entirety.

What is claimed is:

1. A method of identifying a compound for the treatment of cancer, said method comprising:
a) providing one or more targets and one or more components of the riboproteome known to associate with the target,
b) contacting the combined materials of a) with a compound, and
c) monitoring an alteration in the target or the component of the riboproteome,
wherein a modulation in the association between the target and the component of the riboproteome identifies the compound as a potential therapeutic for the treatment of cancer.

2. The method of claim 1, wherein the alteration is seen in both the target and the component of the riboproteome.

3. The method of claim 1, wherein the alteration is in the association between the target and the component of the riboproteome.

4. The method of claim 1, wherein the cancer is a cancer resulting from a change in the level of association between the target and the component of the riboproteome.

5. The method of claim 4, wherein the cancer resulting from the change in the level of the association between the target and the component of the riboproteome is selected from the group consisting of: acute myeloid leukemia, adenoid cystic carcinoma, bladder cancer, bladder urothelial carcinoma, brain lower grade glioma, breast invasive carcinoma, cervical squamous cell carcinoma and endocervical adenocarcinoma, colon and rectum adenocarcinoma, glioblastoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large b-cell lymphoma, ovarian serous cystadenocarcinoma, pancreatic adenocarcinoma, prostate adenocarcinoma, sarcoma, skin cutaneous melanoma, stomach adenocarcinoma, thyroid carcinoma, and uterine corpus endometrial carcinoma.

6. The method of claim 1, wherein the one or more targets is selected from the group consisting of: a RNA binding protein, a metabolic enzyme, a cell adhesion molecule, a component of the proteasome, a component of the ubiquitination machinery, a component of the neddylation machinery, and a signaling protein.

7. The method of claim 6, wherein the one or more targets is a target listed in Table 1 or Table 2.

8. The method of claim 1, wherein the one or more components of the riboproteome is a component listed in Table 3 or Table 4.

9. The method of claim 1, wherein the compound is a small molecule, an RNAi agent, a soluble polypeptide, an antibody, or an antigen-binding fragment.

10. The method of claim 9, wherein the compound specifically interacts with the one or more targets.

11. The method of claim 10, wherein the compound inhibits or promotes the activity of the one or more targets.

12. The method of claim 9, wherein the compound specifically interacts with the one or more components of the riboproteome.

13. The method of claim 9, wherein the compound interacts with a secondary target to modulate the association between the target and the component of the riboproteome.

14. A method of diagnosing a subject as having, or having a predisposition to a particular type of cancer, said method comprising
a) determining an association between one or more targets and one or more components of the riboproteome in the subject, and
b) comparing the level of association to a normal reference, wherein a change in the level of association diagnoses the subject as having a particular type of cancer, and
c) treating the subject for the particular type of cancer by administering a compound that modulates the association between the target and the component of the riboproteome.

15. The method of claim 14, wherein the cancer is a cancer resulting from the change in the level of association between the target and the component of the riboproteome.

16. The method of claim 15, wherein the cancer resulting from the change in the level of association between the target and the component of the riboproteome is selected from the group consisting of: acute myeloid leukemia, adenoid cystic carcinoma, bladder cancer, bladder urothelial carcinoma, brain lower grade glioma, breast invasive carcinoma, cervical squamous cell carcinoma and endocervical adenocarcinoma, colon and rectum adenocarcinoma, glioblastoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large b-cell lymphoma, ovarian serous cystadenocarcinoma, pancreatic adenocarcinoma, prostate adenocarcinoma, sarcoma, skin cutaneous melanoma, stomach adenocarcinoma, thyroid carcinoma, and uterine corpus endometrial carcinoma.

17. The method of claim 14, wherein the one or more targets is selected from the group consisting of: a RNA binding protein, a metabolic enzyme, a cell adhesion molecule, a component of the proteasome, a component of the ubiquitination machinery, a component of the neddylation machinery, and a signaling protein.

18. The method of claim 17, wherein the one or more targets is a target listed in Table 1 or Table 2.

19. The method of claim 14, wherein the one or more components of the riboproteome is a component listed in Table 3 or Table 4.

20. The method of claim 14, wherein the compound is a small molecule, an RNAi agent, a soluble polypeptide, an antibody, or an antigen-binding fragment.

21. The method of claim 20, wherein the compound specifically interacts with the one or more targets.

22. The method of claim 21, wherein the compound inhibits or promotes the activity of the one or more targets.

23. The method of claim 20, wherein the compound specifically interacts with the one or more components of the riboproteome.

24. The method of claim 20, wherein the compound interacts with a secondary target to modulate the association between the target and the component of the riboproteome.

25. The method of claim 14, wherein the one or more targets is myristoylated alanine-rich C kinase substrate (MARCKS) or La-related protein 1 (LARP1).

26. The method of claim 14, wherein the particular type of cancer is prostate adenocarcinoma.

27. A method of diagnosing a subject as having, or having a predisposition to a particular type of cancer or predicting a response to treatment of a particular type of cancer, said method comprising:
a) obtaining a riboproteome profile from the subject,
b) identifying changes in the riboproteome profile compared to a normal reference, and
c) treating the subject or providing an improved treatment regime based on the changes in the riboproteome profile.
